(19) United States
(12) Patent Application Publication
(10) Pub. No.: US 2024/0200083 A1
(43) Pub. Date: Jun. 20, 2024

(54) PLASMID SYSTEM WITHOUT SELECTABLE MARKERS AND PRODUCTION METHOD THEREOF

(71) Applicant: Nanjing GenScript Biotech Co., Ltd., Nanjing (CN)

(72) Inventors: Fan Zhou, Nanjing (CN); Yifan Li, Beijing (CN); Lu Zhang, Nanjing (CN); Hong Li, Nanjing (CN)

(73) Assignee: Nanjing GenScript Biotech Co., Ltd., Nanjing, JS (CN)

(21) Appl. No.: 18/286,880

(22) PCT Filed: Nov. 25, 2021
(86) PCT No.: PCT/CN2021/133141

§ 371 Date: Oct. 13, 2023

(30) Foreign Application Priority Data
Apr. 16, 2021 (CN) 202110413747.X

(51) Int. Cl. C12N 15/64; C12N 15/52; C12N 15/70; C12Q 1/68

(52) U.S. Cl. CPC C12N 15/64; C12N 15/52; C12N 15/70; C12Q 1/68; C12N 2800/30

(57) ABSTRACT

The present invention provides a precursor plasmid for preparing a plasmid without selectable markers, including: 1) a replication original site; 2) a selectable marker gene; 3) a target gene or a cloning site for inserting the target gene; and 4) paired recombination sites. The paired recombination sites enable the precursor plasmid to perform self-recombination in the presence of recombinase to form a molecule of daughter plasmid and a molecule of circular double-stranded DNA. The daughter plasmid includes the replication original site and the cloning site or target gene. The circular double-stranded DNA includes the selectable marker gene. The present invention also provides a method for preparing a plasmid without selectable markers by using the precursor plasmid. The prepared plasmid without selectable markers can be used in the field of gene and cell therapy as a DNA delivery vector or a virus packaging plasmid vector to improve the safety of the plasmid.

### TECHNICAL FIELD

The present invention relates to plasmids, and in particular, to a precursor plasmid for preparing a marker-free plasmid. The present invention also relates to a method for preparing the marker-free plasmid and a kit for preparing the marker-free plasmid.

### BACKGROUND

In recent years, gene and cell therapy technology has gradually become a new important method for treatment of human diseases. Safe and efficient DNA delivery vectors enable the gene and cell therapy to play a greater role in the prevention and treatment of human diseases in the future. In the past two decades, non-viral delivery systems based on plasmid vectors and virus packaging/delivery systems based on plasmids have received extensive attention in gene defect repair, disease treatment and prevention, etc. The improvement of the safety, stability, and yield of plasmids and the reduction of cytotoxicity have always been the key research direction of plasmids in the field of gene and cell therapy.

Plasmid DNA molecules used in gene therapy usually have a modular structure, including a eukaryotic transcription unit and a prokaryotic replication unit. In addition to the sequences required for DNA replication, conventional plasmids usually carry at least one antibiotic resistant gene to ensure that the positive clone is facilitated in screening and stable heredity during cloning. Commonly used selectable markers include ampicillin, kanamycin, chloramphenicol, neomycin, tetracycline, etc. [1]. However, when applied to gene therapy, plasmid DNA may be absorbed by bacteria that exist on the surface of the respiratory or digestive tract, and the antibiotic resistant gene carried on the plasmid may cause side effects such as antibiotic resistance in patients. Therefore, the disadvantageous side effects of traditional plasmid prompt the development of plasmids without antibiotic resistance genes.

At present, the widely used plasmids without antibiotic resistance genes mainly include the following: 1) Minicircles (circular DNA molecules). The minicircle is derived from the conventional plasmid containing recombination sites by self-recombination in E. coli to form a circular DNA molecule containing only a target gene sequence and a small plasmid containing an antibiotic resistance gene with the replication capability. The minicircle has the shortest plasmid backbone sequence, but has a cumbersome production process and low yield and purity due to the lack of replication capability and the limitation of recombination efficiency and purification means, making it difficult to produce on a large scale [2]. 2) Plasmids based on RNA-OUT selection technology. Such plasmids inhibit the expression of the negative selectable gene SacB of the host strain by coding a stretch of RNA-OUT antisense strand, so that the transformed positive clones can grow on a sucrose-containing culture medium for screening [3]. This screening system is easy to scale up for production. The plasmid backbone contains a DNA replication element and an RNA-OUT element, and is more complex in the sequence (450-500 bp) than the minicircle.

### SUMMARY

According to an aspect, the present invention provides a precursor plasmid, including:
1) a replication original site;
2) a selectable marker gene;
3) a target gene or a cloning site for inserting the target gene; and
4) paired recombination sites, where
the paired recombination sites enable the precursor plasmid to perform self-recombination in the presence of recombinase to form a molecule of daughter plasmid without the selectable marker gene and a molecule of circular double-stranded DNA;
the daughter plasmid includes the replication original site and the target gene, or includes the replication original site and the cloning site; and the circular double-stranded DNA includes the selectable marker gene.

In some embodiments, the sequences of the paired recombination sites are in the same direction.

In some embodiments, the replication original site is adjacent to the target gene or the cloning site, the paired recombination sites are respectively adjacent to the upstream and downstream of the replication original site and the target gene, or the paired recombination sites are respectively adjacent to the upstream and downstream of the replication original site and the cloning site. In some preferred embodiments, the replication original site is adjacent to the target gene or the cloning site, the paired recombination sites are respectively located at both ends of the “replication original site—target gene”, or the paired recombination sites are respectively located at both ends of the “replication original site—cloning site”. In some other preferred embodiments, the replication original site is located at one end of the target gene or the cloning site, the paired recombination sites are respectively located at both ends of the “replication original site—target gene”, or the paired recombination sites are respectively located at both ends of the “replication original site—the cloning site”.

In some embodiments, the paired recombination sites are the loxP sequence in the same direction, the FRT sequence in the same direction, or the attB/attP sequence in the same direction.

In some embodiments, the paired recombination sites are the lox71 sequence and the lox66 sequence in the same direction. In some embodiments, the paired recombination sites are the attB sequence and the attP sequence in the same direction.

In some embodiments, the precursor plasmid may include one or more paired recombination sites. In an embodiment, the precursor plasmid includes one paired recombination sites. In another embodiment, the precursor plasmid includes at least two paired recombination sites.

In some embodiments, the replication original site may be derived from a replication original site of bacteria or bacteriophage. In some specific embodiments, the replication original site is a replication original site of bacteria. In some preferred embodiments, the replication original site is selected from a replication original site for the pUC, a replication original site for the pMB1 and derivatives thereof, a replication original site for the ColE1, and a replication original site for the R6Kγ. In some embodiments, the replication original site includes the following sequences: nucleotide sequences as set forth in SEQ ID NOs: 43-46 and nucleotide sequences that have at least 800%, 85%, 90%, 91%, 92%, 93%, 94%, 95%, 96%, 97%, 98%, or 99% identity with the nucleotide sequences as set forth in SEQ ID NOs: 43-46 and can function as the replication origin. In some other embodiments, the replication original site includes a nucleotide sequence that has at least 80%, 85%, 90%, 91%, 92%, 93%, 94%, 95%, 96%, 97%, 98%, or 99% identity with a nucleotide sequence as set forth in SEQ ID NO: 43, 44, 45, or 46 and can function as the replication origin. In some embodiments, the replication original site includes a nucleotide sequence as set forth in SEQ ID NO: 43, 44, 45, or 46. In a specific embodiment, the nucleotide sequence of the replication original site is as set forth in SEQ ID NO: 43, 44, 45, or 46.

In some embodiments, the precursor plasmid further includes the following sequences: the repressor of primer (rop) gene sequence, the coding sequence of endonuclease, and the coding sequence of plasmid replication accessory protein. The endonuclease may be I-SceI. The plasmid replication accessory protein may be n protein. In some other embodiments, the circular double-stranded DNA includes one or more of the following sequences: a rop gene sequence, a coding sequence of endonuclease, and a coding sequence of plasmid replication accessory protein.

In some embodiments, the precursor plasmid includes other plasmid backbone sequences. In some other embodiments, the circular double-stranded DNA includes the other plasmid backbone sequences.

In some embodiments, the selectable marker gene is an antibiotic resistant gene. In some other embodiments, the selectable marker gene is selected from the genes resistant to ampicillin, kanamycin, chloramphenicol, neomycin, or tetracycline.

In some embodiments, the precursor plasmid further includes the coding gene of recombinase. In some other embodiments, the circular double-stranded DNA may include the coding gene of the recombinase. In some embodiments, the precursor plasmid can express the recombinase under suitable conditions. The suitable conditions may be any conditions that enable the precursor plasmid to express the recombinase, for example, may include suitable host cells, growth conditions, induction conditions, and the like. In addition to the coding gene of the recombinase, the precursor plasmid may also contain elements necessary for expressing the recombinase, such as a promoter, an enhancer, and a terminator.

In some embodiments, the precursor plasmid includes a nucleotide sequence that has at least 80%, 85%, 90%, 91%, 92%, 93%, 94%, 95%, 96%, 97%, 98%, or 99% identity with a nucleotide sequence as set forth in SEQ ID NO: 8, 34, 39-42, or 47. In some embodiments, the precursor plasmid includes a nucleotide sequence as set forth in SEQ ID NO: 8, 34, 39-42, or 47. In an embodiment, the nucleotide sequence of the precursor plasmid is as set forth in SEQ ID NO: 8, 34, 3942, or 47.

According to another aspect, the present invention provides use of the foregoing precursor plasmid in preparing a daughter plasmid without selectable marker gene.

According to another aspect, the present invention provides a method for preparing a daughter plasmid without selectable marker gene, including: 1) introducing the foregoing precursor plasmid into a host cell that can express or can help express the recombinase, and screening out host cells that express the selectable marker gene; 2) culturing the host cells screened out in step 1) to allow the recombinase to be expressed in the host cells, and culturing the host cells and screening out host cells that do not express the selectable marker gene; and 3) culturing the host cells screened out in step 2) and extracting plasmids to obtain the daughter plasmid.

In some embodiments, the preparation method further includes the step of obtaining or preparing the foregoing precursor plasmid before step 1).

In some embodiments, the precursor plasmid includes a replication original site, a selectable marker gene, a target gene, and paired recombination sites. The paired recombination sites enable the precursor plasmid to perform self-recombination in the presence of recombinase to form a molecule of the daughter plasmid and a molecule of circular double-stranded DNA. The daughter plasmid includes the replication original site and the target gene. The circular double-stranded DNA includes the selectable marker gene. In some embodiments, the daughter plasmid does not include an antibiotic resistant gene.

In some embodiments, the sequences of the paired recombination sites are in the same direction.

In some embodiments, the paired recombination sites are the loxP sequence in the same direction, and the recombinase is the Cre recombinase; the paired recombination sites are the FRT sequence in the same direction, and the recombinase is the Flp recombinase; or the paired recombination sites are the attB/attP sequence in the same direction, and the recombinase is the PhiC31 recombinase.

In some embodiments, the paired recombination sites are the lox71 sequence and the lox66 sequence in the same direction, and the recombinase is the Cre recombinase. In some other embodiments, the paired recombination sites are the FRT sequence in the same direction, and the recombinase is the Flp recombinase. In some embodiments, the paired recombination sites are the attB/attP sequence in the same direction, and the recombinase is the PhiC31 recombinase.

In some embodiments, the replication original site is derived from a replication original site of bacteria or bacteriophage. In some specific embodiments, the replication original site is selected from a replication original site for the pUC, a replication original site for the pMB1 and derivatives thereof, a replication original site for the ColE1, and a replication original site for the R6Kγ. In some other embodiments, the replication original site includes the following sequences: nucleotide sequences as set forth in SEQ ID NOs: 43-46 and nucleotide sequences that have at least 80%, 85%, 90%, 93%, 94%, 95%, 96%, 97%, 98%, or 99% identity with the nucleotide sequences as set forth in SEQ ID NOs: 43-46 and can function as the replication origin.

In some embodiments, the selectable marker gene is an antibiotic resistant gene. In some other embodiments, the selectable marker gene is selected from genes resistant to ampicillin, kanamycin, chloramphenicol, neomycin, or tetracycline.

In some embodiments, the precursor plasmid includes the coding gene of the recombinase, and the precursor plasmid can express the recombinase in the host cell.

In some embodiments, the precursor plasmid includes a nucleotide sequence that has at least 80%, 85%, 90%, 91%, 92%, 93%, 94%, 95%, 96%, 97%, 98%, or 99% identity with a nucleotide sequence as set forth in SEQ ID NO: 8, 34, 39-42, or 47. In some other embodiments, the precursor plasmid includes a nucleotide sequence as set forth in SEQ ID NO: 8, 34, 39-42, or 47. In an embodiment, the nucleotide sequence of the precursor plasmid is as set forth in SEQ ID NO: 8, 34, 39-42, or 47.

In some embodiments, the host cell includes the coding gene of the recombinase in the genome thereof, or the host cell includes an expression vector containing the coding gene of the recombinase.

In some embodiments, the recombinase is inducibly expressed in the host cell. In some other specific embodiments, the host cell includes an inducible promoter, and the inducible promoter enables the recombinase to be inducibly expressed in the presence of an inducer. For example, the inducible promoter may be a lactose promoter (lactose operon), an arabinose promoter (arabinose operon), a temperature-induced promoter, a metal ion-induced promoter, and the like. In some other embodiments, the recombinase gene is integrated into the host cell genome through the plasmid containing the inducible promoter and the recombinase coding gene. The integration method includes, but is not limited to, the CRISPR/Cas9 method.

In some embodiments, the expression vector includes a conditionally induced loss-type plasmid replication element. In some specific embodiments, the expression vector includes a conditionally induced loss-type replicon, such as a temperature-sensitive replicon and a metal ion replicon. Under the induction condition, the expression vector loses the replication function and is gradually lost in the host cell.

In some embodiments, the host cell is E. coli. For example, the host cell may be E. coli JM108, TOP10, DH5α, GT115, pir1, pir2, etc., and other modified strains derived from related strains.

All the definitions and technical features of the foregoing precursor plasmid are suitable for the precursor plasmid in the method for preparing a daughter plasmid, which are all introduced herein. Details are not described again.

Further, the present invention includes a daughter plasmid obtained by the foregoing preparation method.

According to another aspect, the present invention provides a kit for preparing a daughter plasmid without selectable marker gene, including the foregoing precursor plasmid.

In some embodiments, the kit further includes a host cell that can express or can help express the recombinase. The host cell may be E coli. For example, the host cell may be E. coli JM108, TOP10, DH5α, GT115, pir1, pir2, etc., and other modified strains derived from related strains.

In some embodiments, in the precursor plasmid, the paired recombination sites are the loxP sequence in the same direction, and the recombinase is the Cre recombinase; the paired recombination sites are the FRT sequence in the same direction, and the recombinase is the Flp recombinase; or the paired recombination sites are the attB/attP sequence in the same direction, and the recombinase is the phiC31 recombinase. In an embodiment, the paired recombination sites are the lox71 sequence and the lox66 sequence in the same direction, and the recombinase is the Cre recombinase. In another embodiment, the paired recombination sites are the FRT sequence in the same direction, and the recombinase is the Flp recombinase.

In some embodiments, the host cell includes the coding gene of the recombinase in the genome thereof, or the host cell includes an expression vector containing the coding gene of the recombinase.

In some embodiments, the recombinase is inducibly expressed in the host cell. In some other specific embodiments, the host cell includes an inducible promoter, and the inducible promoter enables the recombinase to be inducibly expressed in the presence of an inducer. For example, the inducible promoter may be a lactose promoter (lactose operon), an arabinose promoter (arabinose operon), a temperature-induced promoter, a metal ion-induced promoter, and the like. The recombinase gene is integrated into the host cell genome through the plasmid containing the inducible promoter and the recombinase coding gene. The integration method includes, but is not limited to, the CRISPR/Cas9 method.

In some embodiments, the expression vector includes a conditionally induced loss-type plasmid replication element. In some specific embodiments, the expression vector includes a conditionally induced loss-type replicon, such as a temperature-sensitive replicon and a metal ion replicon.

In some embodiments, a target gene is inserted into the cloning site, so that the daughter plasmid formed after recombination contains the target gene.

All the definitions and technical features of the foregoing precursor plasmid are suitable for the precursor plasmid in the foregoing kit, which are all introduced herein. Details are not described again.

According to still another aspect, the present invention provides a daughter plasmid, including a replication original site and a target gene, where the daughter plasmid does not include an antibiotic resistant gene. In some embodiments, the daughter plasmid does not include a selectable marker gene. In some embodiments, the daughter plasmid is basically composed of a replication original site and a target gene.

In some embodiments, the replication original site is derived from a replication original site of bacteria or bacteriophage. In some other embodiments, the replication original site is selected from a replication original site for the pUC, a replication original site for the pMB1 and derivatives thereof, a replication original site for the ColE1, and a replication original site for the R6Kγ. In some other embodiments, the replication original site includes the following sequences: nucleotide sequences as set forth in SEQ ID NOs: 43-46 and nucleotide sequences that have at least 80%, 85%, 90%, 93%, 94%, 95%, 96%, 97%, 98%, or 99% identity with the nucleotide sequences as set forth in SEQ ID NOs: 43-46 and can function as the replication origin.

In some embodiments, the target gene includes the following sequences: a promoter, a coding gene of an expressed protein, and a terminator. In some other embodiments, the target gene includes the coding gene of the expressed protein, and may further include a promoter, a terminator, an enhancer, and the like.

In some embodiments, the daughter plasmid can replicate in the host cell. The host cell may be E. coli. For example, the host cell may be E. coli JM108, TOP10, DH5α, GT115, pir1, pir2, etc., and other modified strains derived from related strains. The daughter plasmid is fermented and cultured in the host cell, and then obtained by plasmid extraction. This method can produce daughter plasmids on a large scale to meet production requirements.

According to still another aspect, the present invention also provides a host cell including the foregoing daughter plasmid, where the daughter plasmid can replicate in the host cell. In some embodiments, the daughter plasmid can be obtained from the host cell by culture, collection, and extraction.

All the definitions and technical features of the foregoing daughter plasmid are suitable for the daughter plasmid in the host cell, which are all introduced herein. Details are not described again.

According to still another aspect, the present invention also provides a composition, including the foregoing daughter plasmid, where the content of the daughter plasmid is at least 80%, 81%, 82%, 83%, 84%, 85%, 86%, 87%, 88%, 89%, 90%, 91%, 92%, 93%, 94%, 95%, 96%, 97%, 98%, or 99%.

In some embodiments, a method for determining the content of the daughter plasmid is an NGS analysis method or a gel electrophoresis imaging analysis method. In an embodiment, the content of the daughter plasmid is determined by the proportion of the sequence of the daughter plasmid in the composition by performing NGS analysis on the composition. In some embodiments, the content of the daughter plasmid in the composition determined by the NGS analysis method is at least 80%, 85%, 88%, 90%, 91%, 92%, 93%, 94%, 95%, 96%, 97%, 98%, or 99%. In an embodiment, the content of the daughter plasmid in the composition determined by the NGS analysis method is at least 98%. In another embodiment, the content of the daughter plasmid in the composition determined by the NGS analysis method is at least 99%. In some embodiments, the composition uses the NGS analysis method, where the content of the resistant gene sequence is less than 0.1%, 0.08%, 0.06%, 0.04%, 0.02%, 0.01%, or 0.008%. In some other embodiments, the composition uses the NGS analysis method, where the content of the host cell genome is less than 10%, 8%, 7%, 6%, 5%, 4%, 3%, 2%, or 1%. In some other embodiments, the composition is analyzed by the NGS analysis method, where the target daughter plasmid accounted for more than 90% of the composition, the residue of the resistance gene sequence is less than 0.02%, and the content of the host cell genome is less than 5%.

In another embodiment, the content of the daughter plasmid is determined by the band brightness of the corresponding position of the daughter plasmid in the supercoiled monomer form in gel imaging by performing gel electophoresis imaging analysis on the composition. In some embodiments, the content of the daughter plasmid in the composition determined by the gel electrophoresis imaging analysis method is at least 80%, 81%, 82%, 83%, 84%, 85%, 86%, 87%, 88%, 89%, 90%, 91%, 92%, 93%, 94%, 95%, 96%, 97%, 98%, or 99%. In an embodiment, the content of the daughter plasmid in the composition determined by the gel electrophoresis imaging analysis method is at least 90%.

The plasmid without selectable markers prepared by using the precursor plasmid can be used in the field of gene and cell therapy as a DNA delivery vector or a virus packaging plasmid vector to improve the safety of the plasmid. Compared with minicircles, the production of the plasmid without antibiotic resistance genes involved in the present invention has the following advantages: 1) the plasmid has a high yield, and the plasmid without selectable markers involved in the present invention includes a replication element, which can replicate and amplify autonomously, and has a yield equivalent to that of conventional plasmids; 2) the plasmid product has a high purity, the precursor plasmid is recombined in the host bacteria, strains that do not contain the precursor plasmid are obtained by screening antibiotic-sensitive strains, the circular DNA containing the antibiotic resistant gene produced by recombination is naturally metabolized during bacterial culture due to the lack of replication capability, so that strains that only carry plasmids without resistance selectable markers can be obtained, and high-purity plasmid DNA can be prepared with the strains; and 3) the preparation process is easy to scale up, and the strains carrying plasmids without antibiotic resistance genes prepared by one-time recombination can be used for fermentation production of the same plasmid in different batches without repeated recombination preparation.

### BRIEF DESCRIPTION OF THE DRAWINGS

FIG. 1 is a schematic structural diagram of a precursor plasmid, where ori: replication original site; RS: recombination site; Redundant backbone include AB^{R} gene: a backbone sequence including an antibiotic resistant gene; and Target Gene: a target gene.

FIG. 2 is an electrophoresis gel image of cre fragment amplification, where lane 1 is a marker, and lane 2 and lane 3 are two parallel cre gene amplification products.

FIG. 3 is an electrophoresis gel image of preparing the pSC101-araBAD linear vector, where lane 1 is a marker, and lanes 2 to 5 are four parallel digestion products.

FIG. 4 is an electrophoresis gel image of the PCR verification result of pSC101-araBAD-cre(Amp^{R}) plasmid, where lanes 1 to 12 are PCR products from 12 colonies, and lane 13 is a marker.

FIG. 5 shows a sanger sequencing result of the pSC101-araBAD-cre(Amp^{R}) plasmid, where each arrow above represents an actual sequence obtained by one sequencing reaction, and two sequencing reactions completely cover the cre gene fragment to be sequenced.

FIG. 6 is an electrophoresis gel image of fragment preparation of the pSC101(ts)-araBAD-cre plasmid, where lane 1 is a marker, lane 2 and lane 3 are amplified pSC101 on (ts) fragment products, and lane 4 and lane 5 are pSC101-araBAD-cre(Amp^{R}) plasmid digestion products.

FIG. 7 shows a sanger sequencing result of the pSC101-araBAD-cre(ts) plasmid.

FIG. 8 is an electrophoresis gel image of the PCR verification result of the JM108 genetically modified bacteria, where lane 1 is a marker, lane 2 is a PCR product of the wild-type JM108 strain, and lane 3 is a PCR product of the colony with the araBAD-cre fragment knocked in.

FIG. 9 shows a sanger sequencing result of the JM108 genetically modified bacteria.

FIG. 10 is a gel image of an FLP fragment amplification result, where lane 1 is a marker, and lane 2 and lane 3 are amplification products of the FLP gene.

FIG. 11 is an electrophoresis gel image of the prepared pSC101(ts)-araBAD linear vector, where lane 1 is a marker, and lanes 2 to 5 are pSC101(ts)-araBAD-cre digestion products (that are parallel).

FIG. 12 shows a sanger sequencing result of pSC101(ts)-araBAD-flp, where each arrow above represents an actual sequence obtained by one sequencing reaction, and two sequencing reactions completely cover the FLP fragment to be sequenced.

FIG. 13 is a schematic diagram of a precursor plasmid vector containing the lox71/lox66 recombination site, where backbone include pMB1 ori: a backbone sequence including the pMB1 replication original site; MCS: multiple cloning site; and Redundant backbone include Kan^{R} Gene: a backbone sequence including the kanamycin resistant gene.

FIG. 14 is an electrophoresis gel image of the digestion verification result of the pMF9-loxP plasmid after, where lane 1 is a marker, and lanes 2 to 5 are pMF9-loxP digestion products (that are parallel).

FIG. 15 is an electrophoresis gel image of the prepared pMF9-loxP linear vector, where lane 1 is a marker, and lane 2 and lane 3 are pMF9-loxP digestion products (that are parallel).

FIG. 16 shows a sanger sequencing result of pMF9-loxP-5.5 kb, where each arrow above represents an actual sequence obtained by one sequencing reaction, and nine sequencing reactions completely cover the 5.5 kb fragment to be sequenced.

FIG. 17 is a schematic structural diagram of the pMF65-loxP precursor empty vector plasmid containing the specific recombination site loxp in the same direction, where pMB1 ori: pMB1 replication original site; MCS: multiple cloning site; and Redundant backbone include Kan^{R} Gene: a backbone sequence including the kanamycin resistant gene.

FIG. 18 is an electrophoresis gel image of the prepared pUC57(Kan^{R}) linear vector template, where lane 1 is a marker, and lanes 2 and 3 are pUC57(Kan^{R}) plasmid digestion products (that are parallel).

FIG. 19 is an electrophoresis gel image of fragment preparation of the pMF65-loxp precursor empty vector plasmid, where lane 1 is a marker, lane 2 and lane 3 are amplification products of pUC57 (Kan^{R}) containing the pMB1 replicon fragment (that are parallel), and lane 4 and lane 5 are amplification products of the fragment containing Kan^{R} selectable marker gene and loxp71/66 sites (that are parallel).

FIG. 20 shows a sequencing result of the pMF65-loxp precursor empty vector plasmid, where each arrow above represents an actual sequence obtained by one sequencing reaction, and six sequencing reactions completely cover the entire plasmid to be sequenced.

FIG. 21 is a schematic structural diagram of the pMF65-loxP-RFP precursor plasmid containing the specific recombination site loxp in the same direction, where pMB1 ori: pMB1 replication original site; RFP cassette: the target gene inserted; and Redundant backbone include Kan^{R} Gene: a backbone sequence including the kanamycin resistant gene.

FIG. 22 is an electrophoresis gel image of fragment preparation of the pMF65-loxp-RFP precursor plasmid, where lane 1 is a marker, lane 2 and lane 3 are the RFP amplification fragment, and lane 4 and lane 5 are the pMF65-loxp vector fragment.

FIG. 23 shows a sequencing result of the pMF65-loxp-RFP precursor plasmid, where each arrow above represents an actual sequence obtained by one sequencing reaction, and five sequencing reactions completely cover the entire plasmid to be sequenced.

FIG. 24 is a schematic structural diagram of the pMF7-loxP-RFP precursor plasmid containing the specific recombination site loxp in the same direction, where pUC ori: pUC replication original site; RFP cassette: the target gene inserted; Redundant backbone include Kan^{R} Gene: a backbone sequence including the kanamycin resistant gene; and rop: rop gene.

FIG. 25 is a schematic structural diagram of the pMF5-loxP-RFP precursor plasmid containing the specific recombination site loxp in the same direction, where R6Kγ ori: R6Kγ replication original site; RFP cassette: the target gene inserted; and Redundant backbone include Kan^{R} Gene: a backbone sequence including the kanamycin resistant gene.

FIG. 26 is a schematic structural diagram of the precursor plasmid containing the specific recombination site FRT in the same direction, where backbone include pMB1 ori: a backbone sequence including the pMB1 replication original site; RFP cassette: an expression element containing the target gene; and Redundant backbone include Kan^{R} Gene: a backbone sequence including the kanamycin resistant gene.

FIG. 27 is an electrophoresis gel image of amplification of the FRT-RFP fragment and vector, where lane 1 is a marker, lane 2 and lane 3 are amplification products of the FRT-RFP fragment (that are parallel), and lane 4 and lane 5 are amplification products of the pMF9-loxP-RFP vector fragment (that are parallel).

FIG. 28 shows a sanger sequencing result of pMF9-FRT-RFP, where each arrow above represents an actual sequence obtained by one sequencing reaction, and four sequencing reactions completely cover the fragment to be sequenced.

FIG. 29 is an electrophoresis gel image of verification result of induced Cre-loxP 5.5 kb plasmid recombination, where lane 1 is an unrecombined precursor plasmid; lane 2 is a product after 1 h recombination: the main band is the generated plasmid without resistance gene, and the band corresponding to the precursor plasmid is significantly weakened; lane 4 and lane 5 are linear DNA bands of the plasmids at lane 1 and lane 2 that are digested; and lane 3 is a marker.

FIG. 30 shows a result of antibiotic plate screening, where the left panel is a screening result of antibiotic-sensitive strains, the 10 selected clones did not grow colonies on an antibiotic plate; and the test tubes #1-5 in the right panel show the growth status of the colonies 1-5 in the left panel in a non-antibiotic culture medium, and the colonies grow normally (the white dots on the plate in the left panel are holes in the culture medium caused by a monoclonal dotting operation).

FIG. 31 is an electrophoresis gel image of plasmid verification after antibiotic plate screening, where lanes #1-5 on the left are plasmid products extracted from the test tubes #1-5 in FIG. 30 (5 parallel clones), middle lane 6 is a marker, and lanes #1-5 on the right are digestion products of the plasmids at the lanes #1-5 on the left.

FIG. 32 is an electrophoresis gel image of verification result of induced pMF65-loxp-RFP plasmid recombination, where lane 1 is a DL3000 Marker; lane 2 is an unrecombined precursor plasmid; and lane 3 is a product after 1 h recombination: the main band is the generated plasmid without resistance gene, and the band corresponding to the precursor plasmid is significantly weakened.

FIG. 33 is an electrophoresis gel image of plasmid verification after antibiotic plate screening, where lane 1 is a marker, lane 2 is a control of the precursor plasmid without induced recombination, and lane 3 is a daughter plasmid obtained after 1 h induced recombination.

FIG. 34 shows a sequencing result of the plasmid after antibiotic plate screening, where each arrow above represents an actual sequence obtained by one sequencing reaction, and three sequencing reactions completely cover the entire plasmid to be sequenced; and the hollow arrow shows that the actual sequence does not have the map sequence by sequencing, and the gene is missing.

FIG. 35 is an electrophoresis gel image of verification result of induced pMF7-loxp-RFP plasmid recombination, where lane 1 is a Kb Ladder Marker; lane 2 is an unrecombined precursor plasmid; and lane 3 is a product after 1 h recombination: the main band is the generated plasmid without resistance gene, and the band corresponding to the precursor plasmid is significantly weakened.

FIG. 36 is an electrophoresis gel image of plasmid verification after antibiotic plate screening, where lane 1 is a marker, lanes 1 to 7 are purified plasmid products (7 parallel plasmid products).

FIG. 37 shows a sequencing result of the plasmid after antibiotic plate screening, where each arrow above represents an actual sequence obtained by one sequencing reaction, and three sequencing reactions completely cover the entire plasmid to be sequenced; and the hollow arrow shows that the actual sequence does not have the map sequence by sequencing, and the gene is missing.

FIG. 38 is an electrophoresis gel image of verification result of the digested plasmid after FLP-FRT recombination, where lanes 1 to 7 on the left and right show no induced recombination, 2 h induced recombination, 5 h induced recombination, 8 h induced recombination, 2 h induction and overnight at 37° C., 5 h induction and overnight at 37° C., and 8 h induction and overnight at 37° C. respectively, the lanes 1 to 7 on the left are plasmid products, the lanes 1 to 7 on the right are plasmid digestion products, and middle lane 8 is a marker.

FIG. 39 shows screening of non-resistant strains, where the left panel is an image of the growth of 10 colonies after induced recombination for 2 h, 5 h, and 8 h respectively on a kanamycin plate and an ampicillin plate, and the right panel is an image of the growth of 5 colonies after induced recombination for 5 h cultured in the test tube with antibiotic-free culture medium.

FIG. 40 is an electrophoresis gel image of verification result of plasmid from antibiotic-sensitive strains obtained by resistance screening, where different lanes are parallel clones of the same strain selected under the condition of induced recombination for 2 h, 5 h, and 8 h, and lane 1 is a marker.

FIG. 41 is an electrophoresis gel image of verification result of induced pMF5-loxp-RFP plasmid recombination, where lane 1 is a Kb Ladder Marker; lane 2 is unrecombined precursor plasmids (1): pSC101(ts)-araBAD-cre and pMF5-loxp-RFP precursor plasmid; lane 3 is products after 1 h recombination (2): pSC101(ts)-araBAD-cre, pMF5-loxp-RFP precursor plasmid, circular double-stranded DNA with Kan^{R} gene, and pMF5-loxp-RFP; and the band corresponding to the pMF5-loxp-RFP precursor plasmid is significantly weakened.

FIG. 42 is an electrophoresis gel image of verification result of plasmid from antibiotic-sensitive strains obtained by resistance screening, where lanes 1-5 are 5 parallel clones of the same strain, and lane 6 is a marker.

FIG. 43 shows a sequencing result of the plasmid extracted from antibiotic-sensitive strains obtained by resistance screening, where referring to the precursor plasmid sequence (the black line at the bottom marked with the plasmid element), the region covered by the solid arrow at the top represents the actual sequence sequenced; and the result shows that only the plasmid replication element and the target gene sequence are detected in the plasmid product, and the antibiotic resistant gene Kan^{R} and other redundant vector sequences are not detected, which is consistent with the expected sequence of the marker-free plasmid product.

FIG. 44 is an electrophoresis gel image of verification result of the digestion product of pMF9-5.5 kb plasmid, where lane 1 is the pMF9-5.5 kb plasmid product, lane 2 is a digestion product of the pMF9-5.5 kb plasmid, and lane 3 is a marker.

FIG. 45 shows a sanger sequencing result of the large-scale extracted pMF9-5.5 kb plasmids.

FIG. 46 is an electrophoresis gel image of verification result of the digestion product of pMF65-RFP plasmid, where lane 1 is the pMF65-RFP plasmid product, lane 2 is a digestion product of the pMF65-RFP plasmid, and lane 3 is a marker.

FIG. 47 shows a sanger sequencing result of the large-scale extracted pMF65-RFP plasmids.

FIG. 48 is an electrophoresis gel image of verification result of the digestion product of pMF7-RFP plasmid, where lane 1 is the pMF7-RFP plasmid product, lane 2 is a digestion product of the pMF7-RFP plasmid, and lane 3 is a marker.

FIG. 49 shows a sanger sequencing result of the large-scale extracted pMF7-RFP plasmids.

### DETAILED DESCRIPTION

Unless otherwise specified, all technical and scientific terms used herein have the meanings as commonly understood by a person of ordinary skill in the art. For the purpose of facilitating the understanding of the technical solutions provided herein, some technical terms are briefly described below.

Where the context permits or unless otherwise specified, the term “comprise” or “include” herein covers “consist of” and/or “essentially consist of” and the meaning of the term conforms to the provisions of the patent law.

“Plasmid” or “plasmid vector” can be used interchangeably herein, and refers to a circular DNA molecule with a replication original site (or referred to as a DNA replication element) to have an autonomous replication capability in a host cell. The plasmid may be a natural plasmid or a modified plasmid. The plasmid may include a selectable marker gene, such as an antibiotic resistant gene, so that host cells containing the plasmid can grow under specific culture conditions, while host cells that do not contain the plasmid cannot grow normally under the specific culture conditions. For example, host cells (such as E coli) containing a plasmid with a tetracycline resistant gene can grow in a culture medium containing tetracycline, and host cells that do not contain or lose the plasmid cannot grow or grow inhibited in the culture medium containing tetracycline. Therefore, the use of the selectable marker gene allows the technician to learn which host cells contain the desired plasmid to complete the screening. The plasmid or the modified plasmid may also include a cloning site to facilitate the insertion of a target gene. The target gene can replicate with the plasmid after inserted into the plasmid through the cloning site to achieve the amplification of the target gene; or the plasmid can be used for the expression of the target gene in a host cell when constructed as an expression plasmid. The cloning site may be a single cloning site or a multiple cloning site. To facilitate experimental operations, the multiple cloning site is usually preferred. The multiple cloning site herein refers to a DNA segment containing multiple sites recognized by restriction endonucleases or other endonucleases (such as homing endonucleases). For example, the restriction endonuclease may be Ahd I, AclI, HindIII, SspI, MiuCI, Tsp509I, PciI, AgeI, BspMI, BfuAI, SexAI, MluI, BceAI, Nde I, or EcoR I.

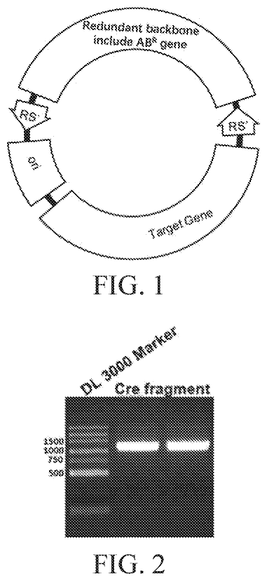

“Precursor plasmid” herein refers to a parent plasmid used to generate a target plasmid/daughter plasmid (such as a plasmid without a selectable marker gene). The “precursor plasmid” includes the following elements: a replication original site, which enables the plasmid to replicate in a host cell to increase or maintain the quantity of the precursor plasmid or the generated target plasmid in the host cell; a selectable marker gene, used for screening host cells; a target gene or a cloning site for inserting the target gene; and paired recombination sites, used for recombination in the presence of the corresponding recombinase to eliminate the sequence between the paired recombination sites. To generate a plasmid without the selectable marker gene, the selectable marker gene is usually inserted between the paired recombination sites (referring to FIG. 1). In this way, during recombination, a precursor plasmid molecule forms two circular DNA molecules. One circular DNA molecule contains a replication original site and a target gene or cloning site, and does not contain a selectable marker gene. Correspondingly, the other circular DNA molecule contains a selectable marker gene, and does not contain a replication original site and a cloning site. The former is also referred to herein as a daughter plasmid or a target plasmid, that is, a plasmid that does not contain a selectable marker gene. For the purpose of the present invention, a cloning site encompasses the cloning site itself, or a cloning site with a target gene inserted. Alternatively, it can be considered that a precursor plasmid not containing the target gene at the cloning site (or referred to as “platform plasmid” or “precursor empty vector plasmid”) is a parent plasmid of a precursor plasmid containing the target gene at the cloning site, and they are all included in the meaning of “precursor plasmid” herein.

“Daughter plasmid”, also referred to as “mini plasmid”, “target plasmid”, and “plasmid without selectable markers” herein, refers to a sequence containing a replication original site and a cloning site and/or a target gene produced by recombination of a precursor plasmid containing a recombination site (such as the loxP sequence in the same direction) in the presence of a recombinase (such as the Cre recombinase). In some embodiments herein, the daughter plasmid includes a replication original site and a cloning site and/or a target gene sequence, but does not include a selectable marker gene. In some other embodiments herein, the backbone sequence of the daughter plasmid refers to a sequence not containing a target gene or a cloning site with a sequence length of less than 1000 bp, less than 900 bp, less than 800 bp, less than 700 bp, less than 600 bp, less than 500 bp, less than 400 bp, less than 300 bp, or less than 200 bp. In some specific embodiments, the length of the backbone sequence of the daughter plasmid may be 878 bp, 864 bp, 708 bp, 623 bp, or 429 bp.

A “daughter plasmid” is a plasmid with specific structure and characteristics as described in the specification and protected by the claims of this application. That is, it is a special plasmid, so that the definition can exist independently of “precursor plasmid” or “parent plasmid”. The inventor does not rule out that this special plasmid (i.e., daughter plasmid) can be obtained by methods other than the present invention, for example, instead of the use of a precursor plasmid, this special plasmid is prepared by other methods, but as long as the obtained plasmid meets the structure and/or definition of the “daughter plasmid” herein, it still falls within the protection scope of this application.

The “recombination site” herein refers to a nucleotide sequence that can be specifically recognized by the corresponding recombinase. When the precursor plasmid contains paired recombination sites in the same direction, under the action of the corresponding recombinase, recombination occurs between the recombination sites in the precursor plasmid, resulting in the generation of a molecule of daughter plasmid and a molecule of circular DNA. The sequence directions of the paired recombination sites in the precursor plasmid are the same. Under the action of the corresponding recombinase, the DNA fragment between the two recombination sites will be deleted to generate two DNA molecules. In some embodiments, the recombination site is the loxP sequence, and the corresponding recombinase is the Cre recombinase. In some other embodiments, the recombination site is the FRT sequence, and the corresponding recombinase is the Flp recombinase. In still some embodiments, the recombination site is the attB/attP sequence, and the recombinase is the PhiC31 recombinase. A person skilled in the art may understand that the recombination sites used in the precursor plasmid only need to cause the sequence (including the selectable marker gene) between them to be removed from the precursor plasmid during recombination and the remaining sequence to form the daughter plasmid. Therefore, these recombination sites are not limited to the specific sequences mentioned herein, but may be their mutants or other recombination sites. For example, the recombination site loxP mutant may be lox75, lox44, lox76, lox43, lox72, lox78, lox65, lox511, lox5171, or lox2272. The recombination site FRT may be wild-type or may be mutant, such as FRT3 and FRT5. In a specific embodiment herein, the recombination sites loxP are the lox71 and lox66 sequences, and the nucleotide sequences are as set forth in SEQ ID NOs: 25 and 26. In another specific embodiment, the nucleotide sequence of the recombination site FRT is as set forth in SEQ ID NO: 27. In a specific embodiment, the recombination site attB and attP sequences are as set forth in SEQ ID NOs: 48 and 49 respectively.

The term “recombinase” refers to an enzyme involved in the process of gene mapping and recombination. It is responsible for recognizing and cutting specific recombination sites and connecting two molecules involved in recombination. In this specification, the recombinase may be the Cre recombinase, the Flp recombinase, or the phiC31 recombinase. In some embodiments, the coding gene of the recombinase includes a sequence that has at least 80%, 85%, 90%, 91%, 93%, 95%, 97%, or 99% identity with a nucleotide sequence as set forth in SEQ ID NO: 1, 24, or 50. For example, the coding gene of the Cre recombinase includes the nucleotide sequence as set forth in SEQ ID NO: 1, the Flp recombinase includes the nucleotide sequence as set forth in SEQ ID NO: 24, and the phiC31 recombinase includes the nucleotide sequence as set forth in SEQ ID NO: 50.

“Selectable marker” or “selectable marker gene” as used herein refers to a segment of selectable marker gene in a plasmid, vector or cell. The introduction of the gene into cells, especially cultured bacteria or cells, can express proper features for artificial selection. It is a reporter gene used to show whether foreign DNA has been successfully transfected or transformed into cells by other methods in the fields of microorganisms, molecular biology, and genetic engineering. The selectable marker gene is usually an antibiotic resistant gene, or may be some auxotrophic selectable markers such as glucosamine synthetase selectable markers and mannose phosphate isomerase selectable markers, or may be negative selectable markers such as anti-toxic gene or anti-SacB selectable markers. In this specification, the selectable marker gene may be an expression cassette, and may include other sequences for expressing the selectable marker gene, such as a promoter, an enhancer, and a terminator. The SacB gene is a structural gene that encodes levosucrose of Bacillus subtilis. In the presence of sucrose, the expression of SacB in Gram-negative bacteria is toxic.

“Screening stress” or “selection stress” can be used interchangeably. In this specification, it refers to the application of specific substances or conditions to organisms such as host cells in a culture condition (such as a culture medium), so that organisms that adapt to these specific substances or conditions can survive, and organisms that do not adapt are eliminated. For example, in this specification, an antibiotic such as kanamycin or ampicillin is added into the culture medium of the host cells, and this culture medium contains antibiotic selection stress, which can screen out host cells with the corresponding antibiotic resistance.

“Adjacent” herein means that two DNA sequence fragments are located upstream and downstream in the direction of gene replication, and positions of the two are very close. For example, the distance between the positions of the two gene fragments is less than 100 bp, less than 90 bp, less than 80 bp, less than 70 bp, less than 60 bp, less than 50 bp, less than 40 bp, less than 30 bp, less than 20 bp, less than 10 bp, less than 5 bp, less than 3 bp, or less than 1 bp. In some embodiments, the two gene fragments are directly connected. In this specification, the distance between positions of the replication original site and the target gene or cloning site in the direction of replication in the precursor plasmid may be less than 50 bp, less than 45 bp, less than 40 bp, less than 35 bp, less than 30 bp, less than 25 bp, less than 20 bp, less than 15 bp, less than 10 bp, less than 5 bp, less than 3 bp, or less than 1 bp. In some embodiments, the replication original site and the target gene or cloning site are directly connected. In some other embodiments, the distance between the replication original site and the target gene or cloning site in the direction of replication is 30 bp, 25 bp, 20 bp, 15 bp, 14 bp, 13 bp, 12 bp, 11 bp, 10 bp, 9 bp, 8 bp, 7 bp, 6 bp, 5 bp, 4 bp, 3 bp, 2 bp, or 1 bp.

In this specification, “the paired recombination sites are respectively adjacent to the upstream and downstream of the replication original site and the target gene” means that the distance between positions of any one of the paired recombination sites and one of the replication original site and the target gene that is closer to the recombination site may be less than 100 bp, less than 90 bp, less than 80 bp, less than 70 bp, less than 60 bp, less than 50 bp, less than 40 bp, less than 30 bp, less than 20 bp, less than 10 bp, less than 5 bp, less than 3 bp, or less than 1 bp. In some embodiments, one or two of the paired recombination sites are directly connected to the replication original site and the target gene, that is, the distance is 0 bp. In some other embodiments, the distance between positions of any one of the paired recombination sites and one of the replication original site and the target gene that is closer to the recombination site may be 70 bp, 65 bp, 60 bp, 55 bp, 50 bp, 45 bp, 40 bp, 35 bp, 30 bp, 25 bp, 20 bp, 15 bp, 14 bp, 13 bp, 12 bp, 11 bp, 10 bp, 9 bp, 8 bp, 7 bp, 6 bp, 5 bp, 4 bp, 3 bp, 2 bp, or 1 bp. In some embodiments, one of the paired recombination sites is directly connected to the replication original site and the target gene, and the distance between positions of the other of the paired recombination sites and the replication original site or the target gene may be 70 bp, 65 bp, 60 bp, 55 bp, 50 bp, 45 bp, 40 bp, 35 bp, 30 bp, 25 bp, 20 bp, 15 bp, 14 bp, 13 bp, 12 bp, 11 bp, 10 bp, 9 bp, 8 bp, 7 bp, 6 bp, 5 bp, 4 bp, 3 bp, 2 bp, or 1 bp. In some other embodiments, two of the paired recombination sites are both directly connected to the replication original site and the target gene. Similarly. “the paired recombination sites are respectively adjacent to the upstream and downstream of the replication original site and the cloning site” means that the replication original site is adjacent to the cloning site, and the distance between positions of any one of the paired recombination sites and one of the replication original site and the cloning site that is closer to the recombination site may be less than 100 bp, less than 90 bp, less than 80 bp, less than 70 bp, less than 60 bp, less than 50 bp, less than 40 bp, less than 30 bp, less than 20 bp, less than 10 bp, less than 5 bp, less than 3 bp, or less than 1 bp. In some embodiments, one or two of the paired recombination sites are directly connected to the replication original site and the cloning site, that is, the distance is 0 bp. In some other embodiments, the distance between positions of any one of the paired recombination sites and one of the replication original site and the cloning site that is closer to the recombination site may be 70 bp, 65 bp, 60 bp, 55 bp, 50 bp, 45 bp, 40 bp, 35 bp, 30 bp, 25 bp, 20 bp, 15 bp, 14 bp, 13 bp, 12 bp, 11 bp, 10 bp, 9 bp, 8 bp, 7 bp, 6 bp, 5 bp, 4 bp, 3 bp, 2 bp, or 1 bp. In some embodiments, one of the paired recombination sites is directly connected to the replication original site and the cloning site, and the distance between positions of the other of the paired recombination sites and the replication original site or the cloning site may be 70 bp, 65 bp, 60 bp, 55 bp, 50 bp, 45 bp, 40 bp, 35 bp, 30 bp, 25 bp, 20 bp, 15 bp, 14 bp, 13 bp, 12 bp, 11 bp, 10 bp, 9 bp, 8 bp, 7 bp, 6 bp, 5 bp, 4 bp, 3 bp, 2 bp, or 1 bp. In some other embodiments, two of the paired recombination sites are both directly connected to the replication original site and the cloning site. Preferably, in the related plasmid described, “the paired recombination sites are respectively adjacent to the upstream and downstream of the replication original site and the target gene”, and the replication original site is also adjacent to the target gene.

In this specification, the “target gene” may be designed with different nucleotide sequences according to different requirements, may include different element sequences such as a promoter, a coding gene of an expressed protein, and a terminator, and may further include a regulatory element such as an enhancer and polyA. The target gene may also include mRNA gene that encodes protein or peptide antigens and protein or peptide therapeutic agents, mRNA, shRNA, RNA, or microRNA that encodes RNA therapeutic agents, mRNA, shRNA, RNA, or microRNA that encodes RNA vaccines, and the like.

“Host cell” herein refers to a cell in which a plasmid can be maintained and/or replicated, including a prokaryote and a eukaryote, such as bacteria (E. coli), fungi (yeast), insect cells, and mammalian cells. In the method for preparing a plasmid that does not contain a selectable marker gene provided herein, the host cell provides necessary components (such as various enzymes and nucleotide monomer molecules) for the plasmid to replicate therein, and also provides the recombinase required for recombination. The expression of the recombinase in the host cell is preferably controllable expression or inducible expression. In some embodiments, the coding gene of the recombinase is integrated into the genome of the host cell. In some other embodiments, the coding gene of the recombinase is inserted into another expression vector, and the expression vector can be introduced into the host cell together with the precursor plasmid at the front or back. In some embodiments, the coding gene of the recombinase is contained in the precursor plasmid, and can express the recombinase after introduced into the host cell. Regardless of the embodiments, it is preferable to place the recombinase gene under the control of an inducible promoter, so that the technician can control the starting time of recombination.

“Conditionally induced loss-type plasmid replication element” herein means that the replication element loses the replication capability under certain conditions such as high temperature and inducer induction, so that the plasmid is gradually lost during host cell amplification. It may be a temperature-sensitive replication element or a metal ion-induced replication element, such as pSC101 on (ts).

For sequences, the term “identity” refers to the amount of identity between two sequences (such as a query sequence and a reference sequence), which is generally expressed as a percentage. Generally, before the calculation of the identity in percentage terms between two sequences, sequence alignment is first performed and gaps (if any) are introduced. At a certain alignment position, if the bases or amino acids in two sequences are the same, it is considered that the two sequences are identical or matched at the position; and if the bases or amino acids in two sequences are different, it is considered that the two sequences are not identical or mismatched at the position. In some algorithms, the number of matched positions is divided by the total number of positions in an alignment window to obtain the sequence identity. In some other algorithms, the number of gaps and/or the length of the gaps are also taken into account. For the purpose of the present invention, the public alignment software BLAST (available on ncbi.nlm.nih.gov) may be used to obtain the best sequence alignment and calculate the sequence identity between two nucleotide or amino acid sequences with default settings.

In this specification, the “plasmid copy number” refers to the copy number of plasmids in each cell. Single copy means that there is only one of the plasmid in the cell, and multicopy means that there are a plurality of the plasmids in the cell. The increase in the plasmid copy number increases the production yield of plasmids. In this specification, the daughter plasmid copy number is usually 10-20, and the high copy number can reach 500-800, or even higher.

“Plasmid backbone sequence” refers to a sequence used as a template to load the target gene in the genetic engineering plasmid, so that the target gene sequence can be selected and amplified in the host cell. The plasmid backbone sequence may include a plasmid replication functional element and other functional elements related to plasmid production performance. The plasmid backbone sequence may be of eukaryotic, prokaryotic or viral origin without the target gene.

The term “cloning site” refers to any nucleotide or nucleotide sequence that facilitates the linking of one polynucleotide (for example, polynucleotide of the target gene) to another polynucleotide (for example, the cloning vector). Generally, the cloning site includes one or more sites recognized by restriction endonucleases, for example, a multiple cloning site. In some embodiments, the “cloning site” may be a multiple cloning site (also referred to as MCS or polylinker). The multiple cloning site refers to a DNA segment containing multiple sites recognized by restriction endonucleases or other endonucleases (such as homing endonucleases).

polyA refers to a polyadenylation signal or site. Polyadenylation is the addition of a poly(A) tail to an RNA molecule. The polyadenylation signal includes a sequence motif recognized by the RNA cleavage complex. Most human polyadenylation signals contain the AAUAAA sequence and its conserved sequences 5′ and 3′. The commonly used polyA signals come from rabbit β-globin, bovine somatotropin, and SV40 early or SV40 late polyA signals.

The pMB1 replication original site (pMB1 ori) refers to the replication original site from the pMB1 plasmid, or may be a derivative of the replication original site from the pMB1 plasmid. The sequence of the pMB1 replication original site may be as set forth in SEQ ID NO: 44, and the sequence of the derivative of the pMB1 replication original site may be as set forth in SEQ ID NO: 43.

The pUC replication original site (pUC ori) refers to the replication original site of pBR322 derivation with a G to A substitution. It lacks the rop negative regulator and can increase the copy number at an increased temperature. Its sequence may be as set forth in SEQ ID NO: 45.

The R6K replication original site (R6K ori) refers to the region specifically recognized by the R6K Rep protein to initiate DNA replication. It includes, but is not limited to, the R6Kγ replication original site sequence as set forth in SEQ ID NO: 46, and also includes a CpG-free version as described in the U.S. Pat. No. 7,244,609 by Drocourt et al., which is incorporated herein by reference.

The rop gene sequence is a repressor of a primer. In this specification, the precursor plasmid may include the rop gene sequence. The removal of the rop sequence from the daughter plasmid by the recombinase results in a substantial increase in the plasmid copy number.

The term “transfection” refers to a method for delivering nucleic acid into a cell. For example, poly(lactic-co-glycolic acid) (PLGA), ISCOM, liposomes, nonionic surfactant vesicles (niosomes), virosomes, Pluronic block copolymers, chitosan, and other biodegradable polymers, particles, microspheres, calcium phosphate nanoparticles, nanoparticles, nanocapsules, nanospheres, poloxamine nanospheres, electroporation, nucleofection, piezoelectric penetration, acoustic perforation, iontophoresis, ultrasound, membrane rupture mediated by SQZ high-speed cell deformation, corona plasma, blood plasma facilitated delivery, tissue-tolerance blood plasma, laser micro-perforation, shock wave energy, magnetic field, non-contact magnetic penetration, gene gun, microneedles, microgrinding, hydrodynamic delivery, high-pressure tail vein injection, etc. are as known in the art and incorporated herein by reference.

“Terminator” or “transcription terminator” herein refers to a DNA sequence at the end of a marker gene or operon in bacteria for transcription. It may be an intrinsic transcription terminator or Rho-dependent transcription terminator. For an internal terminator, such as the trpA terminator, a hairpin structure is formed in the transcript and destroys the mRNA-DNA-RNA polymerase ternary complex. Alternatively, the Rho-dependent transcription terminator requires the Rho factor (an RNA helicase protein complex) to destroy a nascent mRNA-DNA-RNA polymerase ternary complex. In eukaryotes, the polyA signal is not a “terminator”, but is internal cleavage at the polyA site, which leaves an unblocked 5′ end on the 3′UTR RNA for nuclease digestion. The nuclease catches up with RNA Pol II and causes termination. The termination can be promoted in the short region of the poly A site by transforming the RNA Pol II pause site (eukaryotic transcription terminator). The pause of RNA Pol II allows the nuclease that transforms 3′UTR mRNA after PolyA cleavage to catch up with RNA Pol II at the pause site. A non-limiting list of the eukaryotic transcription terminator known in the art includes C2x4 and a gastrin terminator. The eukaryotic transcription terminator can increase mRNA levels by enhancing proper 3′ end processing of mRNA.

The present invention further provides a method for establishing a recombinase inducible expression system, including a method for preparing an expression vector containing a coding gene of the recombinase and transforming host bacteria, and a method for preparing a host cell genome containing the recombinase gene. The Cre recombinase inducible expression vector is used as an example, the method for preparing an expression vector containing a coding gene of the recombinase includes preparing the cre gene fragment and pSC101-araBAD linear vector; assembling the cre gene fragment and pSC101-araBAD linear vector into the pSC101-araBAD-cre(AmpR) plasmid, and transforming the plasmid into competent cells for culture and verification; and selecting clones correctly verified for plasmid extraction. For example, the method for preparing the Cre recombinase temperature-sensitive inducible expression vector includes: preparing the temperature-sensitive replicon pSC101 on (ts) fragment with the pCP20 plasmid; digesting the pSC101-araBAD-cre(Amp^{R}) plasmid to obtain the araBAD-cre(Amp^{R}) fragment; assembling the pSC101 ori (ts) fragment and the araBAD-cre(Amp^{R}) fragment, transforming the assembled fragment into competent cells for culture and verification; and selecting clones with the verification successful, and extracting the plasmid to obtain the expression vector.

The method for integrating the Cre recombinase into the host cell genome includes: preparing the cre gene fragment and the pSC101-araBAD linear vector; assembling the cre gene fragment and the pSC101-araBAD linear vector into the pSC101-araBAD-cre(Amp^{R}) plasmid, and transforming the plasmid into competent cells for culture and verification; selecting clones correctly verified for plasmid extraction; and performing seamless knock-in of the Cre specific recombinase inducible expression system on the host cell by using the λRed recombination technology and CRISPR/Cas9 technology, and culturing the host cell to obtain inducible expression strains of the Cre recombinase. The method for integrating the Flp recombinase into the host cell includes: preparing the flp gene fragment and the pSC101(ts)-araBAD linear vector; assembling the flp gene fragment and the pSC101(ts)-araBAD into the pSC101(ts)-araBAD-flp plasmid, and transforming the plasmid into competent cells for culture and verification; selecting clones correctly verified for plasmid extraction; and performing seamless knock-in of the Cre specific recombinase inducible expression system on the host cell by using the λRed recombination technology and CRISPR/Cas9 technology, and culturing the host cell (such as E. coli) to obtain inducible expression strains of the Flp recombinase.

For example, in some embodiments, the host cell (such as E coli) integrated with the recombinase coding gene may be first transformed with the precursor plasmid, the E coli containing the precursor plasmid is screened with selectable markers for culture, one part of the culture is exposed to the recombinase expression inducer (the other part of the culture may be stored for later use), and the expression of the recombinase causes the precursor plasmid to undergo recombination between the recombination sites, to form a molecule of daughter plasmid without the selectable marker gene and a molecule of circular DNA containing the selectable marker gene. The host cells continue to be cultured, and the host cells are screened with selectable markers. The host cell containing the precursor plasmid (without recombination) or the circular DNA has the selectable marker feature (for example, antibiotic resistance), while the host cell containing only the daughter plasmid does not have the selectable marker feature (does not have the antibiotic resistance). The host cells that cannot grow on the corresponding antibiotic are selected. The host cells that do not have the selectable marker feature are cultured to obtain the plasmids that do not contain the selectable marker gene.

In the embodiment of introducing the recombinase into the host cell by the expression vector, to prevent the expression vector from contaminating the final product (i.e., the plasmid without selectable marker gene), the expression vector preferably has an inducible loss-type expression, for example, including an inducible loss-type replication element, or using a temperature-sensitive replication element from the pSC101(ts) plasmid.

In some specific embodiments, a method for preparing a plasmid without selectable marker gene provided in the present invention includes the following steps:
(1) constructing a precursor plasmid that can be recombined into a circle DNA;
(2) constructing a site-specific recombinase inducible expression system in E. coli;
(3) transforming the precursor plasmid and screening positive clones in site-specific recombinase expression strains;
(4) culturing cells, and inducing the expression of the recombinase, so that the precursor plasmid undergoes self-recombination, where a molecule of precursor plasmid forms a molecule of mini plasmid without selectable markers containing a target gene and a replication original site and a molecule of circular double-stranded DNA containing a plasmid backbone sequence such as selectable marker gene;
(5) separating and purifying to screen out monoclonal strains without selectable marker gene: and
(6) culturing the cells, and extracting the plasmids.

A person skilled in the art can understand that one or more of foregoing steps may be omitted or the order thereof may be changed according to the situation, as long as the daughter plasmid, i.e., the marker-free plasmid, of the present invention can be obtained. Therefore, in some other specific embodiments, a method for preparing a marker-free plasmid provided in the present invention includes the following steps:
1) introducing the precursor plasmid of the present invention into a host cell that can express or can help express the recombinase, and screening out host cells that express the selectable marker gene;
2) culturing the host cells screened out in step 1) to allow the recombinase to be expressed in the host cells, and culturing the host cells and screening out host cells that do not express the selectable marker gene; and
3) culturing the host cells screened out in step 2) and extracting plasmids to obtain the plasmid without selectable marker gene.

In some embodiments, the preparation method further includes the step of obtaining or preparing the precursor plasmid of the present invention before step 1). In some embodiments, the screening of steps 1) and 2) uses the screening stress corresponding to the selectable marker. In some embodiments, there is no screening stress for the cell culture in step 3).

The recombinase system, whether it is present on the precursor plasmid, in the host cell, or both, may be an inducible expression system or a constitutive expression system.

In this specification, “constitutive expression” means that gene expression (such as the recombinase system) is not affected by time, location, or environment, and has no temporal and spatial specificity. In contrast to the inducible expression, the constitutive expression does not require induction by other factors for stable expression, while the inducible expression requires induction by other factors for expression.

More specifically, a method for preparing a marker-free plasmid provided in the present invention includes the following steps:

(1) Construct a Precursor Plasmid that can be Recombined into a Circle DNA.

The precursor plasmid includes: a replication original site (e.g., a DNA replication element), a selectable marker gene, a pair of specific recombination sites in the same direction, and a target gene. The target gene does not contain a specific recombination site in the same type as the plasmid backbone. Except for the DNA replication element, the plasmid backbone sequence such as the selectable marker gene is located inside the pair of specific recombination sites. The precursor plasmid containing the target gene sequence and DNA replication element can be reconstituted under the action of the site-specific recombinase and divided into two circular double-stranded DNA. One contains the plasmid backbone sequence such as the selectable marker gene, which does not have the replication capability, and is gradually lost during cell amplification. The other is a mini plasmid containing only the DNA replication element and target gene sequence, which can be continuously amplified during cell amplification.

(2) Construct a Site-Specific Recombinase Inducible Expression System in E. coli.

The site-specific recombinase inducible expression system may be constructed on the plasmid vector, and then transformed into the E. coli host cell for expression or integrated onto the E. coli host cell genome for expression. The expression system contains: an inducible prokaryotic transcription promoter, e.g., the araBAD promoter, a site-specific recombinase gene, and a transcription terminator. The site-specific recombinase gene may include: a. the Cre recombinase derived from the P1 bacteriophage Cre-loxP recombination system, corresponding to the pair of specific recombination sites in the same direction in (1) being the loxP sequence (such as lox71/lox66); b. the Flp recombinase derived from the brewer's yeast Flp-FRT recombination system, corresponding to the pair of specific recombination sites in the same direction in (1) being the FRT sequence. When the recombinase expression system is constructed on the plasmid vector, the vector plasmid may be a conditionally induced loss-type plasmid replication element, e.g., the temperature-sensitive replication element pSC101(ts) plasmid, to ensure that the plasmid product without selectable markers does not have the recombinase expression plasmid contamination.

### (3) Transform the Precursor Plasmid and Screen Positive Clones in Site-Specific Recombinase Expression Strains.

The E. coli strain carrying the site-specific recombinase inducible expression system is prepared as competent cells, the precursor plasmid is transformed, and the transformed positive clones are screened by using a selection culture medium corresponding to the selectable marker gene on the precursor plasmid. If the recombinase system is constructed on the plasmid, the recombinase expression plasmid and the precursor plasmid may be co-transformed into conventional E. coli competent cells, and the transformed positive clones are screened through culture by using a double-selection culture medium.

(4) Culture Cells, and Induce the Expression of the Recombinase, so that the Precursor Plasmid Undergoes Self-Recombination.

The transformed positive clones in (3) are selected, and the strain is cultured and enriched in the selection culture medium corresponding to the selectable marker overnight. The cells are washed with a liquid culture medium without selection stress, a culture medium without selection stress containing the inducer is used to resuspend the strain and induce the expression of the recombinase to perform recombination between the two recombination sites carried by the plasmid, and the plasmid backbone sequence such as the selectable marker gene between the recombination sites is deleted to form the mini plasmid without selectable markers.

(5) Separate and Purify to Screen Out Monoclonal Strains without Selectable Marker Gene.

A flat plate without selection stress is streaked with the fully recombined bacterial solution in (4) to separate a single colony. The single colony is copied on a selection culture plate and a non-selection (e.g., antibiotic-free) culture plate that contain the selectable marker gene to culture overnight. The colony on the antibiotic-free plate that cannot grow in the corresponding antibiotic plate is selected for culture to obtain the strain containing only the plasmid without selectable markers.

(6) Culture the Strain Obtained in (5) in a Non-Selection (e.g., Antibiotic-Free) Culture Medium, and Extract the Plasmid, to Obtain a Final Product of the Mini Plasmid without Selectable Markers.

To avoid the safety risks of gene therapy caused by antibiotic resistant genes and achieve high purity and large-scale production of plasmids, the mini plasmid without the selectable marker gene (e.g., antibiotic resistance selectable marker) and with the product plasmid backbone containing only the replication original site (DNA replication element) and the production method thereof provided in the present invention greatly reduces the proportion of bacterial-derived sequences in the product plasmid, and reduces potential safety risks. In addition, it also achieves separation, purification, culture, and amplification of the positive strain containing the plasmid product, and resolves the problem of low purity of the plasmid without selectable markers and difficulty in large-scale production.

The beneficial technical effects of the present invention include, but are not limited to that: the plasmid obtained by the method in the present invention has no antibiotic selectable marker, and has no extra prokaryotic DNA elements except the replication original site; and the plasmid without antibiotic selectable markers has no antibiotic drugs added during production, and is easy to scale up for production to achieve large-scale production.

The plasmid without selectable markers provided in the present invention can be used in the field of gene and cell therapy as a DNA delivery vector or a virus packaging plasmid vector to improve the safety and stability of the plasmid and reduce cytotoxicity.

The technical solution of the present invention is further described in detail below by using the examples and the accompanying drawings. Unless otherwise specified, the methods and materials in the examples described below are commercially available and conventional products. A person skilled in the art to which the present invention belongs will understand that the methods and materials as described below are only exemplary and should not be considered as limiting the scope of the present invention.

### Example 1: Establishment of Cre Recombinase Expression System

In this example, a site-specific recombinase inducible expression system was constructed in the E. coli JM108. This set of system contains an inducible prokaryotic transcription promoter araBAD promoter, a site-specific recombinase gene cre, and a transcription terminator. The system was constructed and expressed in the host bacteria in the following two ways:

1.1 The Cre specific recombinase inducible expression system was constructed on a vector plasmid of the pSC101(ts) replication system to transform the JM108 for expression. The specific steps were as follows:

(1) Preparation of Cre Gene Fragment and pSC101-araBAD Linear Vector

Preparation of cre gene fragment: the cre gene fragment was amplified with primers cre-F and cre-R (the primer sequences were respectively as set forth in SEQ ID NO: 2 and SEQ ID NO: 3), and the cre fragment was synthesized by Nanjing GenScript Biotechnology Co., Ltd. (the cre gene sequence was as set forth in SEQ ID NO: 1). The amplification system and PCR system are shown in Table 1 and Table 2. the Phusion®HF DNA polymerase was commercially available from New England Biotechnology Co., Ltd. under an article number of 10058481. The theoretical size of the cre gene fragment is 1116 bp. Agarose gel electrophoresis was performed on the amplification product, and the fragment size of the amplification product obtained through the electrophoresis was correct (as shown in FIG. 2). The gel was recovered to obtain the cre gene fragment.

TABLE 1
PCR amplification system

| System | Volume | |
|---|---|---|
| Phusion ®HF DNA polymerase (2 U/μL) | 0.5 | μL |
| Phusion ®HF reaction buffer (5×) | 10 | μL |
| dNTP (10 nM) | 1 | μL |
| Primer cre-F (10 pmol) | 1 | μL |
| Primer cre-R (10 pmol) | 1 | μL |
| cre gene template (25 ng/μL) | 0.5 | μL |
| H₂O | 36 | μL |

TABLE 2
PCR procedure

| No. | Procedure |
|---|---|
| 1 | 98° C. 30 s |
| 2 | 98° C. 10 s |
| 3 | 50° C. 30 s |
| 4 | 72° C. 1 min 12 s |
| 5 | To step 2, 30 cycles |
| 6 | 72° C. 5 min |

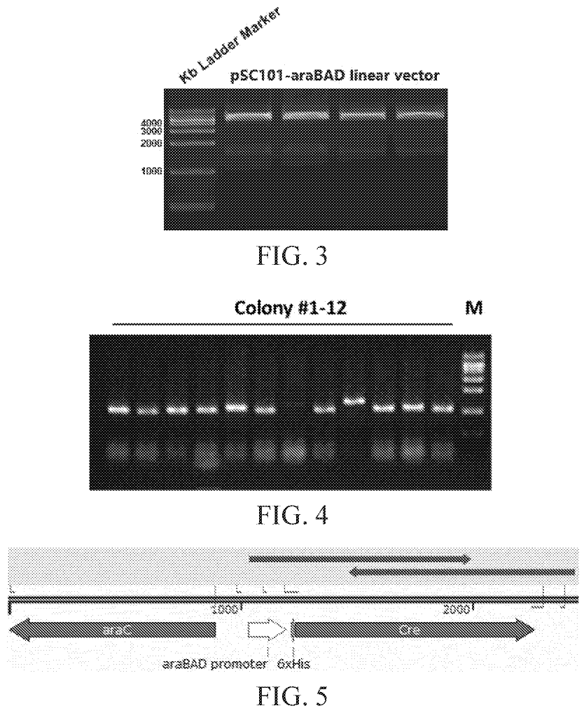

Preparation of pSC101-araBAD linear vector: the pSC101-araBAD linear vector was derived from the pKD46 plasmid with NCBI Sequence ID: AY048746.1. The pKD46 plasmid was digested with EcoR I, and the linear vector was recovered. The digestion system is shown in Table 3. The EcoRI restriction endonuclease was commercially available from New England Biotechnology Co., Ltd. under an article number of 10079659. The digestion system was reacted at 37° C. for 45 min. The digestion product was run on agarose gel for electrophoresis. As shown in FIG. 3, there are two bands after digestion with the size of 4820 bp+1509 bp (4820 bp and 1509 bp respectively), which are consistent with the theoretical values (4820 bp and 1509 bp). The 4820 bp vector fragment was recovered by cutting the gel to obtain the pSC101-araBAD linear vector.

TABLE 3
Digestion system

| System | Volume |
|---|---|
| EcoR I (20 U/μL) | 1 μL |
| 10 × reaction buffer | 5 μL |
| pKD46 plasmid template | 15 μL |
| (130 ng/μL) | |
| H₂O | 29 μL |

(2) Assembly of pSC101-araBAD-Cre(Amp^{R}) Plasmid

The assembly system is shown in Table 4. The Gene Builder™ cloning Kit was commercially available from Nanjing GenScript Biotechnology Co., Ltd. under an article number of C20012009. A reaction liquid was formulated to react at 50° C. for 15 min, and the product was transformed into the DH10b competence and cultured in an incubator at 37° C.

TABLE 4
Assembly system

| System | Volume | |
|---|---|---|
| Gene builder 2 × Master Mix recombinase | 10 | μL |
| (2×) | | |
| cre fragment (75 ng/μL) | 0.4 | μL |
| pSC101-araBAD linear vector (20 ng/μL) | 3 | μL |
| H₂O | 6.6 | μL |

(3) Verification of pSC101-araBAD-Cre(Amp^{R}) Plasmid

A single colony was selected on a transformation plate from the foregoing step, and then transferred into a 48-well plate for culture at 37° C. and 220 rpm overnight. Colony PCR was performed with the primers cre-seq1/cre-seq2 (the sequences of the two primers were as set forth in SEQ ID NO: 4 and SEQ ID NO: 5 respectively, and the size of the amplification fragment was 1069 bp). The product was verified through agarose gel electrophoresis. As shown in FIG. 4, colonies 1-12 are results of the agarose gel electrophoresis of the colony PCR product, and all the other clones show bands except for clone #7, indicating that the assembly may be successful. The positive clones with a correct fragment size were selected to extract plasmids, the plasmids were sent to Nanjing GenScript Biotechnology Co., Ltd. for sanger sequencing, and the sequencing result was correct (as shown in FIG. 5, the tested sequence is completely consistent with the map sequence (the black line in the figure), and there is no gene deletion or mutation, indicating that the plasmid is successfully assembled). The pSC101-araBAD-cre(Amp^{R}) plasmid was successfully assembled.

(4) Construction of Temperature-Sensitive Plasmid pSC101(Ts)-araBAD-Cre

Preparation of temperature-sensitive replicon pSC101 on (ts) fragment: the pSC101 on (ts) fragment (the sequence was as set forth in SEQ ID NO: 23) was PCR-amplified by using the pCP20 plasmid (NCBI Sequence ID: MK178598.1) as a template. The amplification primers pSC101(ts)-F and pSC101(ts)-R are as set forth in SEQ ID NO: 6 and SEQ ID NO: 7 respectively in the primer sequence list. The PCR system and procedure are shown in Table 5 and Table 6. The pSC101 on (ts) fragment product obtained by amplification was run on agarose gel for electrophoresis. As shown in lanes 2 and 3 in FIG. 6, the fragments have a correct size of 1434 bp. The Phusion®HF DNA polymerase was commercially available from New England Biotechnology Co., Ltd. under an article number of 10058481.

Preparation of araBAD-cre(Amp^{R}) fragment: the pSC101-araBAD-cre(Amp^{R}) plasmid prepared in step (3) was treated with Nco I and Not I. The digestion system is shown in Table 7. As shown in lanes 4 and 5 in FIG. 6, the digestion products have a correct fragment size of 4431 bp. The larger fragment was recovered by cutting the gel to obtain araBAD-cre(Amp^{R}). The Nco I and Not I restriction endonucleases were commercially available from New England Biotechnology Co., Ltd. under article numbers of 10080424 and 10046577 respectively.

TABLE 5
PCR amplification system

| System | Volume | |
|---|---|---|
| Phusion ®HF DNA polymerase (2 U/μL) | 0.5 | μL |
| Phusion ®HF reaction buffer (5×) | 10 | μL |
| dNTP (10 nM) | 5 | μL |
| pSC101(ts)-F (10 pmol) | 1 | μL |
| pSC101(ts)-R (10 pmol) | 1 | μL |
| pSC101 ori (ts) fragment (60 ng/μL) | 0.2 | μL |
| H₂O | 32.3 | μL |

TABLE 6
PCR amplification procedure

| No. | Procedure |
|---|---|
| 1 | 98° C. 30 s |
| 2 | 98° C. 10 s |
| 3 | 68° C. 30 s |
| 4 | 72° C. 1 min 30 s |
| 5 | From step 2, 30 cycles |
| 6 | 72° C. 5 min |

TABLE 7
Digestion system

| System | Volume |
|---|---|
| Nco I (20 U/μL) | 1 μL |
| Not I (20 U/μL) | 1 μL |
| 10 × cutsmart ® buffer (NEB | 5 μL |
| cat#B7204S) | |
| pSC101-araBAD-cre(Amp^{R}) | 16 μL |
| plasmid (125 ng/μL) | |
| H₂O | 27 μL |

The pSC101(ts)-araBAD-cre(Amp^{R}) plasmid was assembled. As shown in Table 8, the reaction system was reacted at 50° C. for 15 min. The assembled product was transformed into the DH10b competence and cultured in an incubator at 30° C. The Gene Builder™ cloning Kit was commercially available from Nanjing GenScript Biotechnology Co., Ltd. under an article number of C20012009.

TABLE 8
Assembly system

| System | Volume | |
|---|---|---|
| Gene builder 2 × Master Mix | 10 | μL |
| pSC101 ori (ts) fragment (50 ng/μL) | 1.3 | μL |
| araBAD-cre (Amp^{R}) fragment (13 ng/μL) | 8 | μL |
| H₂O | 0.7 | μL |

PCR verification was performed on the pSC101(ts)-araBAD-cre plasmid. A single colony in a transformation plate in the foregoing step was selected and transferred into a 48-well plate to culture at 30° C. and 220 rpm, 8 clones correctly verified by PCR were randomly selected to extract plasmids for sanger sequencing (provided by Nanjing GenScript Biotechnology Co., Ltd.). Referring to FIG. 7, the sequencing result shows that the tested sequence is completely consistent with the map sequence (the black line in the figure), and there is no gene deletion or mutation, indicating that the plasmid is successfully assembled. The pSC101(ts)-araBAD-cre plasmid was successfully assembled.

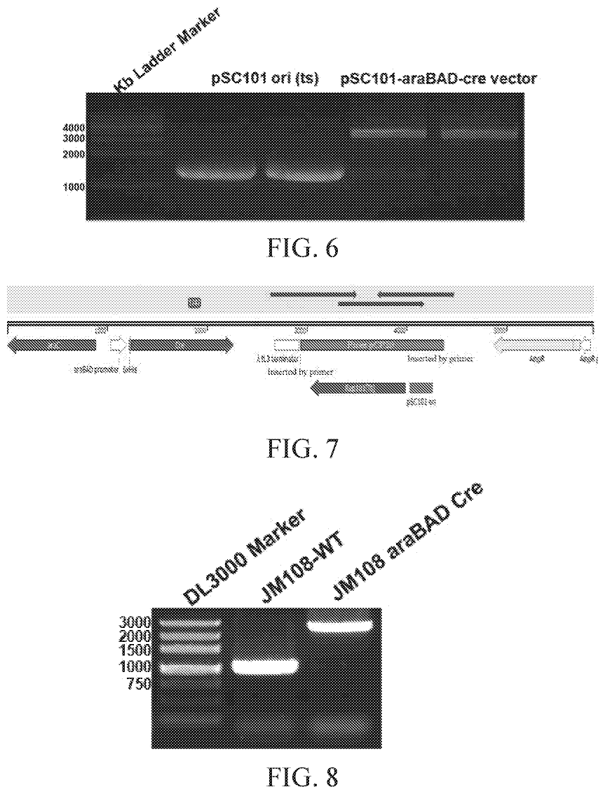

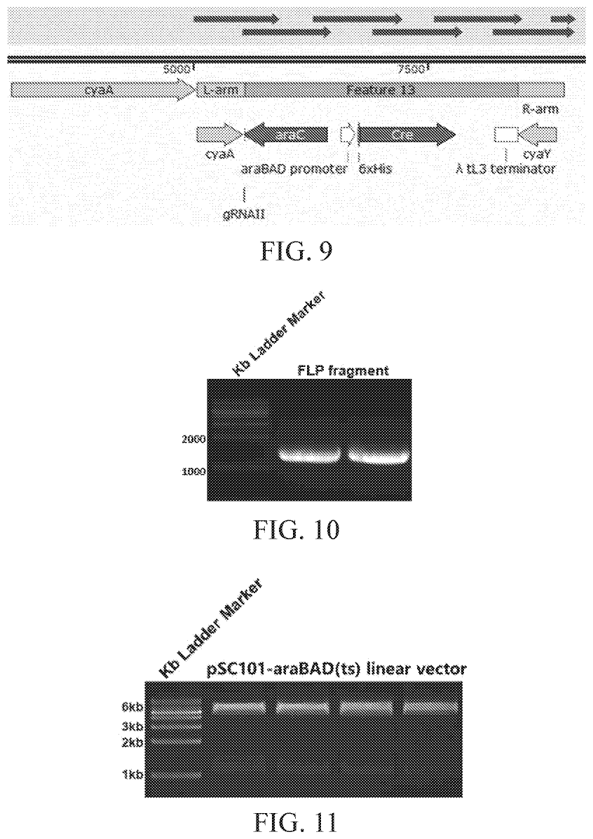

1.2 The inducible expression system of the Cre recombinase was integrated onto the JM108 genome for expression through λRed recombination and CRISPR/Cas9 editing. The strain editing and verification operations 3)-5) were completed by Nanjing GenScript Biotechnology Co., Ltd. (CRISPR products and services (genscript.com.cn)) with the specific steps as follows:
1) The CRISPR/Cas9 specific target site between the cyaA and cyaY genes was designed with a gRNA of 20 bp in length and having sequence specified in SEQ ID NO: 9.
2) The JM108-araBAD-cre(Amp) L-homology arm and JM108-araBAD-cre(Amp)R-homology arm that were located at both sides of the insertion site between the cyaA and cyaY genes were designed with homology arm sequences as set forth in SEQ ID NO: 10 and SEQ ID NO: 11 respectively shown in the sequence list.
3) The Cre specific recombinase inducible expression system (the sequence as set forth in SEQ ID NO: 22) was seamlessly knocked in the E. coli JM108 strain by using the λRed recombination technology and the CRISPR/Cas9 technology.
4) Screening verification by colony PCR and agarose gel electrophoresis was performed on the edited strain (with the primers C4818FK060-6JJF1 and C4818FK060-6JJR1 having the sequences as set forth in SEQ ID NO: 14 and SEQ ID NO: 15 respectively). The theoretical size of the band of the successful knock-in colony PCR on the gel for electrophoresis should be greater than 3000 bp, and the theoretical size of the band of the unsuccessful knock-in colony is 1297 bp. As shown in FIG. 8, the result shows that the size of the band of the successful knock-in colony on the gel for electrophoresis is correct.
5) The sanger sequencing was performed on the clones correctly verified by PCR. As shown in FIG. 9, the result shows that the sequencing result is correct, and there is no mutation.

### Example 2: Establishment of Flp Recombinase Expression System

The establishment of the Flp recombinase inducible expression system may refer to the establishment of the Cre recombinase expression system in Example 1. The Flp recombinase inducible expression system includes an inducible prokaryotic transcription promoter araBAD promoter, a site-specific recombinase gene flp, and a transcription terminator. The system was constructed and expressed in the host bacteria in the following two ways:

2.1 The Flp specific recombinase inducible expression system was constructed on a vector plasmid of the pSC101 ori(ts) temperature-sensitive replication system to transform the JM108 for expression. The specific operation was as follows:

(1) Preparation of flp Gene Fragment and pSC101(Ts)-araBAD Linear Vector

Preparation of flp gene fragment: the flp gene (with the sequence as set forth in SEQ ID NO: 24) was derived from the pCP20 plasmid, and the flp gene was amplified with the primers flp-F and flp-R (with the sequences as set forth in SEQ ID NO: 16 and SEQ ID NO: 17 respectively). The amplification system and PCR system are shown in Table 9 and Table 10. As shown in FIG. 10, the result of the agarose gel electrophoresis shows that the fragment has a correct size of 1333 bp. The gel was recovered. The Phusion®HF DNA polymerase was commercially available from New England Biotechnology Co., Ltd. under an article number of 10058481.

TABLE 9
PCR amplification system

| System | Volume | |
|---|---|---|
| Phusion ®HF DNA polymerase (2 U/μL) | 0.5 | μL |
| Phusion ®HF reaction buffer (5×) | 10 | μL |
| dNTP (10 nM) | 5 | μL |
| Primer flp-F (10 pmol) | 1 | μL |
| Primer flp-(10 pmol)R | 1 | μL |
| pCP20 plasmid (60 ng/μL) | 0.2 | μL |
| H₂O | 32.3 | μL |

TABLE 10
PCR amplification procedure

| No. | Procedure |
|---|---|
| 1 | 98° C. 30 s |
| 2 | 98° C. 10 s |
| 3 | 63° C. 30 s |
| 4 | 72° C. 1 min 12 s |
| 5 | From step 2, 30 cycles |
| 6 | 72° C. 5 min |

Preparation of pSC101(ts)-araBAD linear vector: the pSC101(ts)-araBAD-cre plasmid was digested with Nde I and EcoR I-HF (commercially available from New England Biotechnology Co., Ltd. under article numbers of 10064897 and 10079659 respectively) to prepare the linear vector. The size of the pSC101(ts)-araBAD-cre plasmid was 5876 bp. The digestion product was run on agarose gel for electrophoresis. As shown in FIG. 11, there are two bands after digestion with a size of 4841 bp+1034 bp, which are consistent with the theoretical values. The digestion system is shown in Table 11. The digestion system was reacted at 37° C. for 60 min. The large fragment was recovered by cutting the gel.

TABLE 11
Digestion system

| System | Volume |
|---|---|
| EcoR I-HF (20 U/μL) | 1 μL |
| Nde I (5 U/μL) | 1 μL |
| 10 × cutsmart buffer | 5 μL |
| pSC101(ts)-araBAD-cre plasmid (125 ng/μL) | 10 μL |
| H₂O | 33 μL |

(2) Assembly of pSC101(Ts)-araBAD-Flp Plasmid

The assembly system is shown in Table 12. The assembly system was reacted at 50° C. for 15 min. After the assembly was completed, the assembled product was transformed into the DH10b competence and cultured in an incubator at 30° C. The Gene Builder™ cloning Kit was commercially available from Nanjing GenScript Biotechnology Co., Ltd. under an article number of C20012009.

TABLE 12
Assembly system

| System | Volume |
|---|---|
| Gene builder 2 × Master Mix recombinase working liquid | 10 μL |
| flp fragment (52 ng/μL) | 1 μL |
| pSC101(ts)-araBAD linear vector (13 ng/μL) | 9 μL |

(3) Verification of pSC101(Ts)-araBAD-Flp Plasmid

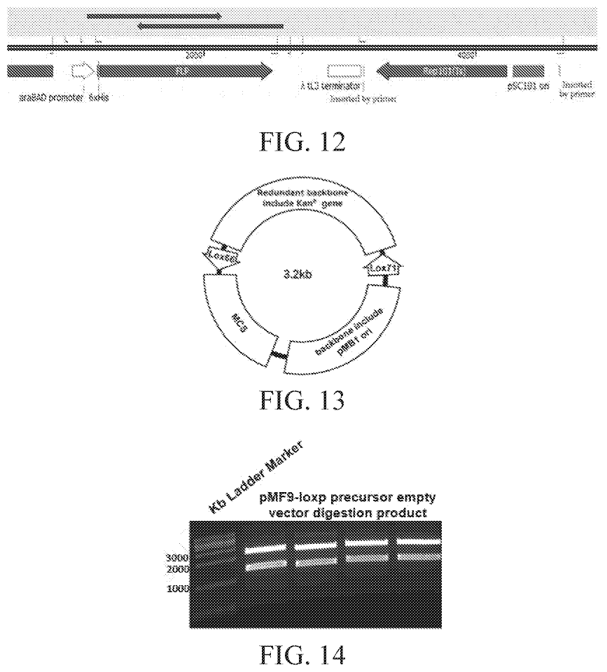

8 single colonies were randomly selected from the foregoing plate and then inoculated in 4 mL of LB liquid culture medium for culture at 30° C. and 220 rpm. The plasmids were extracted for sanger sequencing, and the sequencing was completed by Nanjing GenScript Biotechnology Co., Ltd. Referring to FIG. 12, the sequencing result shows that the tested sequence is completely consistent with the map sequence (the black line in the figure), and there is no gene deletion or mutation, indicating that the plasmid is successfully assembled.

2.2 The inducible expression system of the Flp recombinase was integrated onto the JM108 genome for expression through λRed recombination and CRISPR/Cas9 editing. The specific steps were the same as step 1.2 in Example 1.

### Example 3: Construction of Precursor Plasmid Containing Lox71/Lox66 Recombination Site

3.1 Construction of pMF9-Loxp Precursor Empty Vector Plasmid

The pMF9-loxp precursor empty vector plasmid includes a DNA replication element pMB1 derivative (784 bp, SEQ ID NO: 43), a resistance selectable marker gene KanR, a pair of specific recombination sites lox71/lox66 in the same direction (as shown in the sequence list, the lox71 sequence is as set forth in SEQ ID NO: 25, and the lox66 sequence is as set forth in SEQ ID NO: 26), and a multiple cloning site MCS. The multiple cloning site and the replication element are located between lox71 and lox66 (as shown in FIG. 13), and the order includes, but is not limited to, this example. The plasmid sequence is as set forth in SEQ ID NO: 8, which was synthesized by Nanjing GenScript Biotechnology Co., Ltd. The synthesized pMF9-loxp plasmid was verified by digestion. The restriction endonuclease is Hind III, which was commercially available from New England Biotechnology Co., Ltd. under an article number of 10055811. The digestion system is shown in Table 13. The digestion system was reacted at 37° C. for 45 min. The theoretical size of the target band should be 1176 bp+2015 bp. As shown in FIG. 14, the result of agarose gel electrophoresis of the digestion product shows that the lanes 2-5 are four parallel digestion products with the correct band size, indicating that the obtained plasmid is correct.

TABLE 13
Digestion system

| System | Volume | |
|---|---|---|
| Hind III (5 U/μL) | 0.5 | μL |
| 10 × cutsmart ® buffer (cat# B7204S) | 2 | μL |
| pMF9-loxp plasmid template | 5 | μL |
| H₂O | 12.5 | μL 1 |

3.2 Preparation of pMF9-Loxp Linear Vector

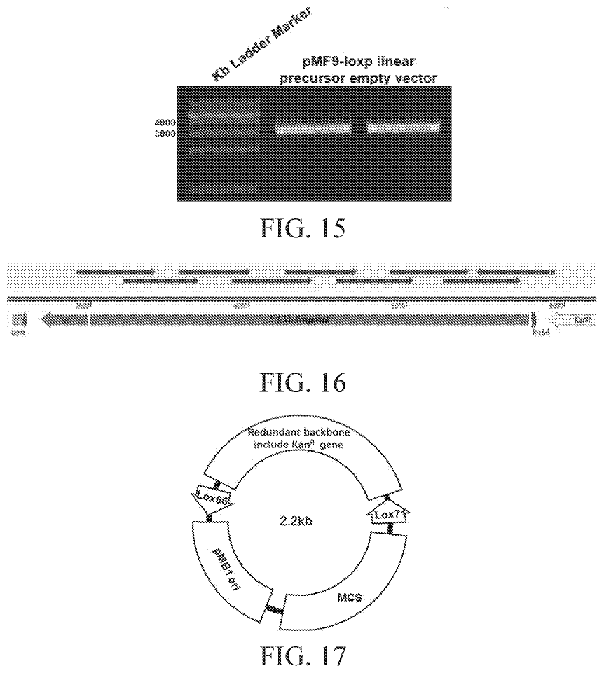

The pMF9-loxp plasmid was digested with BamHI-HF (New England Biotechnology Co., Ltd., 10081013). The digestion system is shown in Table 14. The digestion system was reacted at 37° C. for 45 min. The size of the pMF9-loxp plasmid is 3191 bp. As shown in FIG. 15, the result of agarose gel electrophoresis of the digestion product shows that the lanes 2-3 are two parallel digestion products with the correct band size after digestion. The gel was recovered.

TABLE 14
Digestion system

| System | Volume |
|---|---|
| BamH I-HF (20 U/μL) | 1 μL |
| 10 × cutsmart ® buffer (cat# B7204S) | 5 μL |
| pMF9-loxp plasmid | 35 μL |
| H₂O | 9 μL |

3.3 Assembly of pMF9-Loxp-5.5 kb Precursor Plasmid

The fragment (target gene) with a length of 5.5 kb and without the loxP site was inserted between the loxP sites. The fragment was amplified and recovered using a pair of primers with a homology arm. The sequences of the primers are shown in the sequence list (as set forth in SEQ ID NO: 12 and SEQ ID NO: 13 respectively). The recovered fragment and the pMF9-loxp linear vector prepared in Example 3.2 were assembled. The assembly system is shown in Table 15. The assembly was carried out at 50° C. for 15 min. The assembly system was transformed to JM108. 50 μL/100 μL of the transformed product was poured and cultured at 37° C. overnight. The Gene Builder™ cloning Kit was commercially available from Nanjing GenScript Biotechnology Co., Ltd. under an article number of C20012009.

TABLE 15
Assembly system

| System | Volume |
|---|---|
| Gene builder 2 × Master Mix recombinase | 10 μL |
| 5.5 kb fragment (57 ng/μL) | 5 μL |
| pMF9-loxp linear vector (17 ng/μL) | 5 μL |

3.4 Verification of pMF9-Loxp-5.5 kb Plasmid

The assembled pMF9-loxp-5.5 kb plasmid grew a single colony on the plate after transformation, 8 single clones were randomly selected to be inoculated in 4 mL of LB liquid culture medium for culture at 37° C. and 220 rpm. The plasmids were extracted for sanger sequencing. As shown in FIG. 16, the sanger sequencing result shows that the assembly is successful. The sanger sequencing was completed by Nanjing GenScript Biotechnology Co., Ltd.

### Example 4: Construction of Precursor Plasmid Containing Lox71/Lox66 Recombination Site and pMB1 Shortest Replication Element

4.1 Construction of pMF65-Loxp Precursor Empty Vector Plasmid

Plasmid safety is critical in gene and cell therapy. The sequence on the plasmid backbone is potentially immunogenic, leading to adverse reactions. Shortening the plasmid backbone has always been the optimization direction of plasmids related to gene and cell therapy. The precursor plasmid vector designed in this example can be recombined to obtain a daughter plasmid containing only about 0.65 KB of pMB1 replication element sequence, which reduces the introduction of foreign gene sequences, reduces potential cytotoxicity and immunogenicity, and improves safety and stability.

The plasmid was constructed based on the pUC57(Kan^{R}) plasmid (with the sequence as set forth in SEQ ID NO: 29, synthesized by Nanjing GenScript Biotechnology Co., Ltd.), including a DNA replication element pMB1 (589 bp. SEQ ID NO: 44), a resistance selectable marker gene Kan^{R}, a pair of specific recombination sites lox71/lox66 in the same direction, and a multiple cloning site. The replication element and the cloning site are located between lox71 and lox66 (the plasmid is shown in FIG. 17), and the order includes, but is not limited to, this example.

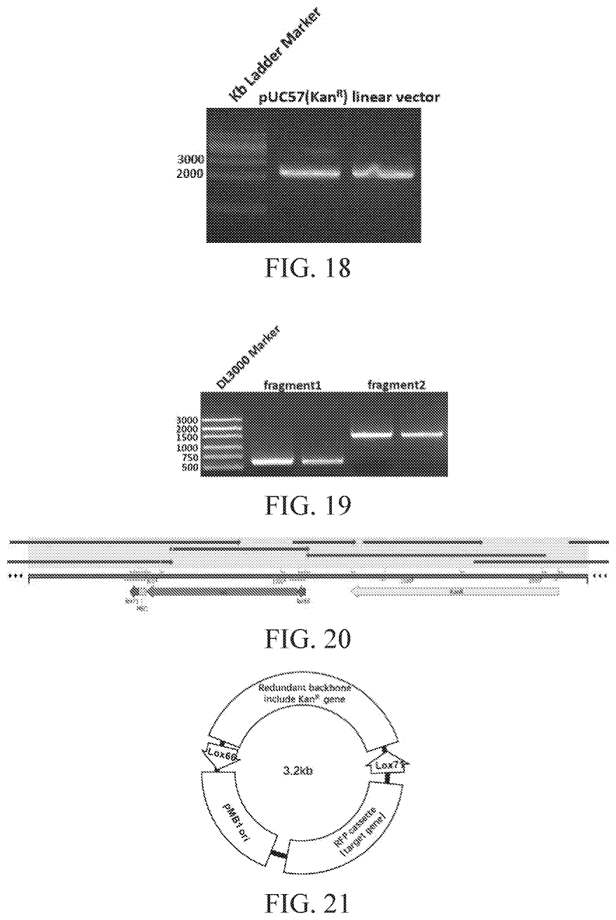

The pUC57(Kan^{R}) plasmid was treated with the restriction endonuclease sall/BamHI at the multiple cloning insertion site, and the pUC57(Kan^{R}) plasmid was digested into a linear vector. The digestion system is shown in Table 16. Referring to FIG. 18, the result shows that the pUC57(Kan^{R}) plasmid has the band size of 2579 bp that is correct. The gel was recovered according to a gel kit. The pUC57(Kan^{R}) fragment containing the pMB1 replicon and the pUC57(Kan^{R}) fragment containing the Kan^{R} selectable marker and loxp71/66 sites were amplified with two pairs of primers respectively. The sequences of the primers for amplifying the fragment containing the pMB1 replicon are as set forth in SEQ ID NO: 30 and SEQ ID NO: 31. The sequences of the primers for amplifying the fragment containing the Kan^{R} selectable marker and loxp71/66 sites are as set forth in SEQ ID NO: 32 and SEQ ID NO: 33. The PCR system and PCR procedure are shown in Table 17 and Table 18 respectively. For the amplification product, as shown in FIG. 19, fragment 1 on the lanes 1 and 2 containing the pMB1 replicon has a size of 668 bp; and fragment 2 on the lanes 3 and 4 containing the Kan^{R} selectable marker and loxp71/66 sites has a size of 1574 bp. The band sizes are both correct. The gel was recovered according to a gel kit. Primer Star GXL DNA polymerase was commercially available from Takara Biomedical Technology (Beijing) Co., Ltd. under an article number of R050A.

TABLE 16
Digestion system

| System | Volume |
|---|---|
| BamH I-HF (20 U/μL) | 1 μL |
| Sall-HF (5 U/μL) | 1 μL |
| 10 × cutsmart ® buffer (cat# B7204S) | 5 μL |
| pUC57(Kan^{R}) plasmid | 28 μL |
| H₂O | 15 μL |

TABLE 17
PCR amplification system

| System | Volume | |
|---|---|---|
| Primer Star GXL polymerase (2 U/μL) | 1 | μL |
| Reaction buffer (5×) | 10 | μL |
| dNTP (10 mM) | 1 | μL |
| DMSO | 5 | μL |
| pUC57-short 1-1/2-1 (50 pmol) | 1 | μL |
| pUC57-short 1-2/2-2 (50 pmol) | 1 | μL |
| pUC57(Kan^{R}) plasmid template (20 ng/μL) | 0.3 | μL |
| H₂O | 30.7 | μL |

TABLE 18
PCR procedure

| No. | Procedure |
|---|---|
| 1 | 98° C. 3 min |
| 2 | 98° C. 30 s |
| 3 | 65° C. 30 s |
| 4 | 68° C. 30 s |
| 5 | From step 2, 30 cycles |
| 6 | 68° C. 5 min |

The pMF65-loxp plasmid was assembled. The system is shown in Table 19. The system was reacted at 50° C. for 15 min. After the assembly was completed, the assembled product was transformed into the JM108 competence and cultured at 37° C. The Gene Builder™ cloning Kit was commercially available from Nanjing GenScript Biotechnology Co., Ltd. under an article number of C20012009.

TABLE 19
Assembly system

| System | Volume | |
|---|---|---|
| Gene builder 2 × Master Mix recombinase | 10 | μL |
| Fragment 2 (21.6 ng/μL) | 4.7 | μL |
| Fragment 1 (34.2 ng/μL) | 1.3 | μL |
| H₂O | 4 | μL |

Verification of pMF65-Loxp Plasmid

The assembled pMF65-loxp plasmid grew a single colony on the plate after transformation. Single clones were selected to be inoculated in 4 mL of LB liquid culture medium for culture at 37° C. and 220 rpm. The plasmids were extracted for sanger sequencing. As shown in FIG. 20, the sanger sequencing result shows that the primer sequencing result covers the complete plasmid sequence without mutation, and the assembly is successful. The sanger sequencing was completed by Nanjing GenScript Biotechnology Co., Ltd.

4.2 Construction of pMF65-Loxp-RFP Precursor Plasmid

The construction of the pMF65-loxp-RFP precursor plasmid refers to the construction of pMF9-loxp-5.5 kb in Example 3. That is, the rfp red chromogenic gene (with the sequence as set forth in SEQ ID NO: 28) was inserted into the MCS of the pMF65-loxp plasmid as a foreign fragment (the structural diagram is shown in FIG. 21).

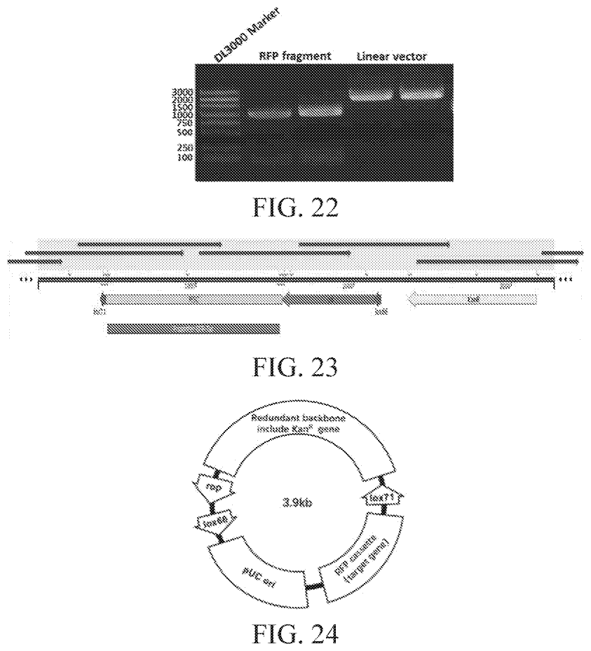

The pMF65-loxp vector and the inserted fragment RFP were amplified by PCR. The size of the vector fragment is 2182 bp, and the size of the RFP fragment is 1150 bp (the sequences of two pairs of primers are as set forth in SEQ ID NO: 35, SEQ ID NO: 36, SEQ ID NO: 37, and SEQ ID NO: 38 respectively). The amplification system is shown in Table 20. The amplification procedure is shown in Table 21. Referring to FIG. 22, the result shows that the lanes 2 and 3 are RFP amplification fragments, the lanes 4 and 5 are pMF65-loxp vector fragments, and the sizes thereof are correct. The gel was recovered according to a gel kit. The assembly was carried out with the Gene Builder™ cloning Kit.

TABLE 20
PCR amplification system

| System | Volume | |
|---|---|---|
| Primer Star GXL polymerase (2 U/μL) | 1 | μL |
| Reaction buffer (5×) | 10 | μL |
| dNTP (10 mM) | 5 | μL |
| DMSO | 5 | μL |
| Short-F/RFP-F (50 pmol) | 1 | μL |
| Short-R/RFP-R (50 pmol) | 1 | μL |
| pUC57(Kan^{R}) plasmid template/pMF9- | 0.3 | μL |
| loxp-RFP plasmid (20 ng/μL) | | |
| H₂O | 26.7 | μL |

TABLE 21
PCR procedure

| No. | Procedure |
|---|---|
| 1 | 98° C. 3 min |
| 2 | 98° C. 30 s |
| 3 | 65° C. 30 s |
| 4 | 68° C. 4 min 30 s |
| 5 | From step 2, 30 cycles |
| 6 | 68° C. 5 min |

The pMF65-loxp-RFP precursor plasmid was assembled. The system is shown in Table 22. The system was reacted at 50° C. for 15 min. After the assembly was completed, the assembled product was transformed into the JM108 competence and cultured at 37° C. The Gene Builder™ cloning Kit was commercially available from Nanjing GenScript Biotechnology Co., Ltd. under an article number of C20012009.

TABLE 22
Assembly system

| System | Volume |
|---|---|
| Gene builder 2 × Master Mix recombinase | 10 μL |
| pMF65-loxp vector fragment (123 ng/μL) | 1.2 μL |
| RFP fragment (59 ng/μL) | 1.3 μL |
| H₂O | 7.6 μL |

Verification of pMF65-Loxp-RFP Precursor Plasmid

The assembled pMF65-loxp-RFP precursor plasmid grew a single colony on the plate after transformation. Single clones were selected to be inoculated in 4 mL of LB liquid culture medium for culture at 37° C. and 220 rpm. The plasmids were extracted for sanger sequencing. As shown in FIG. 23, the sanger sequencing result shows that the primer sequencing result covers the complete plasmid sequence without mutation, and the assembly is successful. The sanger sequencing was completed by Nanjing GenScript Biotechnology Co., Ltd.

### Example 5: Construction of High-Copy Precursor Plasmid Containing Lox71/Lox66 Recombination Site

5.1 Construction of pMF7-Loxp-RFP Plasmid

The plasmid in this example contains a high-copy pUC replicon to improve the yield and purity of the plasmid without selectable markers, simplify the preparation process, reduce production costs, and facilitate large-scale production.

The plasmid was constructed based on the pUC57(Kan^{R}) plasmid (with the sequence as set forth in SEQ ID NO: 29), including a DNA replication element pUC ori (674 bp, SEQ ID NO: 45), a resistance selectable marker gene Kan^{R}, a gene sequence coding the Rop protein, a pair of specific recombination sites lox71/lox66 in the same direction, and a target gene RFP sequence. The replication element and the target gene are located between lox71 and lox66 (the structure is shown in FIG. 24) (the full sequence of the plasmid is as set forth in SEQ ID NO: 39 in the sequence list). The plasmid was synthesized by Gene Department of Nanjing GenScript Biotechnology Co., Ltd. The rop gene sequence is derived from the pBR322 replication element and is a replication control protein, and reduce the copy number of the pUC replication element plasmid. Before induced recombination, the precursor plasmid expresses the Rop protein, and the copy number of the precursor plasmid is inhibited. After induced recombination, the plasmid without resistance selectable markers loses the rop gene sequence to become a high-copy plasmid, which can increase the proportion of the plasmid without resistance selectable markers in the strain after recombination, accelerate the loss of the precursor plasmid, and improve the screening efficiency of the strain containing the plasmid without resistance selectable markers.

### Example 6: Construction of Precursor Plasmid Containing Lox71/Lox66 Recombination Site and R6Kγ Replicon

6.1 Construction of pMF5-Loxp-RFP Precursor Plasmid

This example shows that the method of the present invention is suitable for preparation of the plasmid without selectable markers of different types of replication elements, and the method has universal applicability. R6Kγ on (389 bp, SEQ ID NO: 46) is a replicon different from pMB1 or pUC in Examples 3, 4, and 5. The R6Kγ sequence has a shorter length, which can further shorten the backbone length of the daughter plasmid and reduce cytotoxicity.

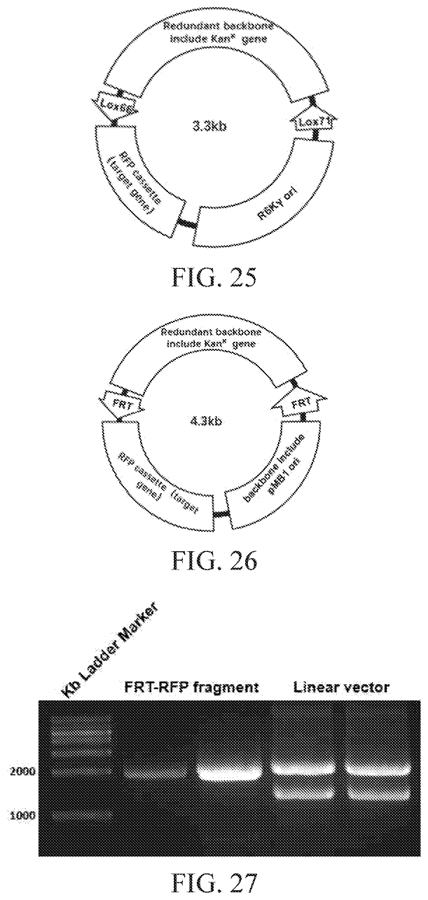

The pMF5-loxP-RFP precursor plasmid includes a DNA replication element R6Kγ ori, a resistance selectable marker gene Kan^{R}, a pair of specific recombination sites lox71/lox66 in the same direction, and a target gene inserted into the sequence RFP. The replication element and the inserted gene are located between lox71 and lox66 (the structure is shown in FIG. 25) (the full sequence of the plasmid is as set forth in SEQ ID NO: 40 in the sequence list). The plasmid was synthesized by Nanjing GenScript Biotechnology Co., Ltd.

### Example 7: Construction of Precursor Plasmid Containing Specific Recombination Site FRT in the Same Direction

### 7.1 Preparation of FRT-RFP Gene Fragment and Vector

The construction of the pMF9-FRT-RFP precursor plasmid (the structure is shown in FIG. 26) is based on the pMF9-loxP-RFP plasmid. The construction of the pMF9-loxP-RFP plasmid refers to the construction method of pMF9-loxP-5.5 kb in Example 3. That is, the rfp red chromogenic gene (with the sequence as set forth in SEQ ID NO: 28) was inserted between the loxP sites of pMF9-loxP-RFP as a target gene. The fragment between the loxP sites of the pMF9-loxP-RFP plasmid was amplified with primers carrying the FRT sequence (the FRT sequence is as set forth in SEQ ID NO: 27 in the sequence list). The specific primer sequences are shown in the sequence list (the FRT-F1 sequence is as set forth in SEQ ID NO: 18, and the FRT-R1 sequence is as set forth in SEQ ID NO: 19). The PCR system and PCR procedure are shown in Table 23 and Table 24 respectively. Gel electrophoresis was performed on the amplification product. As shown in FIG. 27, the sizes of the lanes 2 and 3 are both 2123 bp, which are correct. The gel was recovered according to a gel kit.

Preparation of pMF9 vector fragment: PCR amplification was performed on the vector with the pMF9-loxP-RFP plasmid as a template. The primers FRT-F2 and FRT-R2 are shown in the primer sequence list as set forth in SEQ ID NO: 20 and SEQ ID NO: 21 respectively. The PCR system and PCR procedure are shown in Table 16 and Table 17 respectively. Agarose gel electrophoresis was performed on the amplification product. As shown in lanes 4 and 5 in FIG. 27, the size of the amplification product is 2236 bp. The band with a correct size was cut, and the gel was recovered according to a gel kit. The Phusion®HF DNA polymerase used by PCR was commercially available from New England Biotechnology Co., Ltd. under an article number of 10058481.

TABLE 23
PCR amplification system

| System | Volume | |
|---|---|---|
| Phusion ®HF DNA polymerase (2 U/μL) | 0.5 | μL |
| Phusion ®HF reaction buffer (5×) | 10 | μL |
| dNTP (2.5 mM) | 5 | μL |
| FRT-F 1/2 (10 pmol) | 1 | μL |
| FRT-R1/2 (10 pmol) | 1 | μL |
| pMF9-loxP-RFP plasmid (90 ng/μL) | 0.3 | μL |
| template | | |
| H₂O | 32.2 | μL |

TABLE 24
PCR procedure

| No. | Procedure |
|---|---|
| 1 | 98° C. 30 s |
| 2 | 98° C. 10 s |
| 3 | 68° C./54° C. 30 s |
| 4 | 72° C. 2 min 12 s |
| 5 | From step 2, 30 cycles |
| 6 | 72° C. 5 min |

7.2 The pMF9-FRT-RFP plasmid was assembled. The assembly system is shown in Table 25. The assembly system was reacted at 50° C. for 15 min. After the assembly was completed, the assembled product was transformed into the DH10b competence and cultured at 37° C. The Gene Builder™ cloning Kit was commercially available from Nanjing GenScript Biotechnology Co., Ltd. under an article number of C20012009.

TABLE 25
Assembly system

| System | Volume |
|---|---|
| Gene builder 2 × Master Mix recombinase | 10 μL |
| pMF9 vector fragment (60 ng/μL) | 4 μL |
| FRT-RFP fragment (60 ng/μL) | 4 μL |
| H₂O | 2 μL |

7.3 Verification of pMF9-FRT-RFP Plasmid

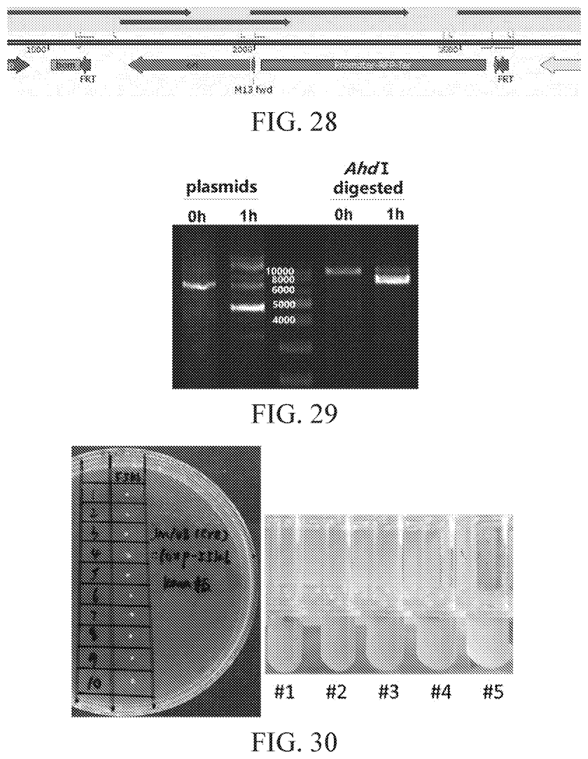

8 single colonies were randomly selected from the transformation plate and sent to Nanjing GenScript Biotechnology Co., Ltd. for sanger sequencing. Referring to FIG. 28, the sequencing result shows that the tested sequence is consistent with the map sequence (the black line in the figure), and there is no gene deletion or mutation, indicating that the plasmid is successfully assembled.

### Example 8: Induced Recombination, Resistance Screening, and Plasmid Purification Based on Single-Plasmid Transformation of pMF9-Loxp Vector

This example describes methods of induced recombination, resistance screening, and plasmid purification based on single-plasmid transformation by using JM108 araBAD-cre+pMF9-loxp-5.5kb as an example, but is not limited to the Cre-loxP recombination system. The specific operations are as follows:

### 8.1 Single-Plasmid Transformation

The competence was prepared with the genetically modified strain JM108 araBAD-cre in Example 1, and the precursor plasmid pMF9-loxp-5.5kb was transformed and then poured on the kanamycin plate. The specific transformation steps are as follows:

The JM108 araBAD-cre competence was placed on ice to melt naturally; 1 μL of the plasmid was added into the competence on ice, and the competence and the plasmid were mixed uniformly, and the mixture was ice-bathed at 42° C. for 30 min, heat shocked for 90s, and then ice-bathed for 3 min. The mixture was added into 800 μL of fresh LB liquid culture medium and incubated in a full-temperature shaking incubator at 37° C. and 220 rpm for 45 min. A proper amount of bacterial solution was poured on a solid plate of LB+Kan+1% glucose and then cultured in an incubator at 37° C. overnight. Single colonies with a normal size were selected for subsequent experiments.

### 8.2 the Addition of Arabinose Induces Cre-loxP Recombination

A certain amount of arabinose was added into the JM108 araBAD-cre pMF9-loxp-5.5 kb culture system to induce expression of the Cre recombinase to achieve the Cre-loxP recombination. The specific operations are as follows:

3-4 JM108 araBAD-cre pMF9-loxp-5.5 kb colonies were selected to be inoculated in 4 mL of liquid culture medium of LB+Kan (kanamycin, 30 μg/mL)+1% glucose for culture at 37° C. and 220 rpm overnight, and the strains were enriched. The strains were collected to be washed twice with antibiotic-free LB and then resuspended with an induction solution (induction solution: LB+2% arabinose). The induced recombination was carried out at 37° C. and 220 rpm for 1 h. antibiotic-free LB solid plate was streaked with 5 μL of recombined bacterial solution to culture in an incubator at 37° C. overnight, and plasmids were extracted from the remaining bacterial solution for verification. The digestion system is shown in Table 26. The Ahd I restriction endonuclease was commercially available from New England Biotechnology Co., Ltd. under an article number of 10079968. Agarose gel electrophoresis was carried out. Taking original plasmids as a control, the electrophoresis result is shown in FIG. 29.

The result shows that: the size of the precursor plasmid pMF9-loxp-5.5 kb is 8720 bp, and after the recombination, the size of the resistant circular DNA generated is 2270 bp and the size of the pMF9-5.5 kb plasmid generated is 6450 bp. It can be learned according to FIG. 29 that the induction for 1 h with the addition of 2% arabinose successfully achieves the Cre-loxP recombination and obtains about 6.5 kb of marker-free plasmid, but there is still a little precursor plasmid residue.

TABLE 26
Digestion system

| System | Volume |
|---|---|
| Ahd I (10 U/μL) | 0.2 μL |
| 10 × cutsmart ® buffer (cat#B7204S) | 1 μL |
| Recombination product (100 ng/μL) | 1 μL |
| H₂O | 7.8 μL |

### 8.3 Kan Resistance Screening and Purification to Obtain Marker-Free Plasmid

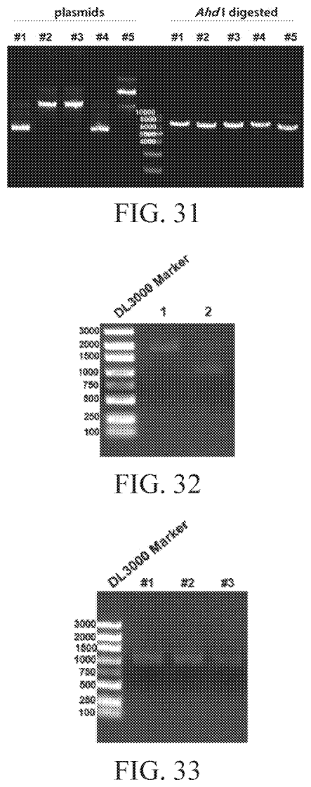

1) After the antibiotic-free plate was streaked with the recombined bacterial solution, 10 single colonies were selected. As shown in the left panel in FIG. 30, the LB+Kan plate was dotted with each colony to culture in an incubator at 37° C. overnight. In addition, as shown in the right panel in FIG. 30, each colony was also inoculated in the LB antibiotic-free liquid culture medium to culture at 37° C. and 220 rpm for resistance screening.
2) 5 colonies that had no colony grown on the LB+Kan plate and had colony normally grown in the LB antibiotic-free liquid culture medium were selected (as shown in FIG. 30), and plasmids were extracted after preservation for plasmid verification.
3) The digestion verification was performed on the extracted plasmids. The digestion system is shown in Table 26. Agarose gel electrophoresis was carried out. The pMF9-5.5 kb plasmid is 6450 bp. The plasmid with a correct plasmid size and a correct digestion product was selected to be the obtained plasmid product without markers (as shown in the lanes #1 and #4 on the left of FIG. 31).

The result of resistance screening plate and plasmid verification shows that the remaining precursor plasmids and resistance genes are eliminated through Kan resistance reverse screening, and the marker-free plasmid is obtained efficiently.

### Example 9: Induced Recombination, Resistance Screening, and Plasmid Purification Based on Single-Plasmid Transformation of Shortest Backbone Precursor Plasmid Containing Only pMB1 Replication Element

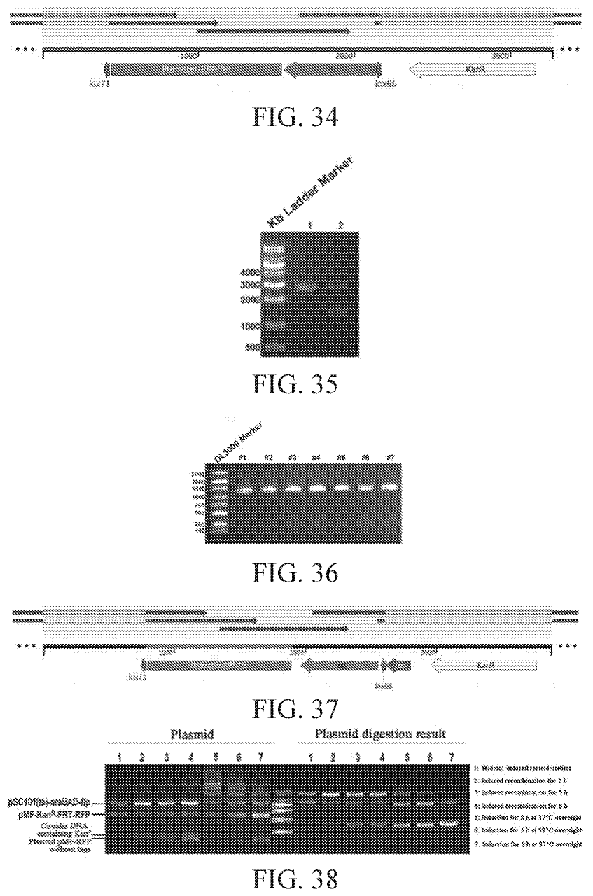

This example tests the feasibility of reducing the pMB1 replication element of the precursor plasmid to the shortest length for the production of the plasmids without selectable markers in the present invention based on the pMF65-loxp-RFP precursor plasmid in Example 4. The specific implementation is the same as Example 8. The recombination of the precursor plasmid was achieved through induction. As shown in FIG. 32, the detection result of gel electrophoresis shows that the lane 2 is a control of the precursor plasmid without induced recombination with a size of 3272 bp, the lane 3 is the MF plasmid obtained by induced recombination for 1 h with a size of 1743 bp, and the band sizes are all correct. The pMF65-RFP plasmid without resistance selectable markers with a correct size was obtained by subsequent resistance screening and purification. As shown in FIG. 33 (3 parallel plasmid products), the result of gel electrophoresis shows that the band has a correct size and is single, which is a haploid band. The sequencing verification was performed on the pMF65-loxp-RFP plasmid. As shown in FIG. 34, the verification result indicates that the recombination is successful.

### Example 10: Induced Recombination, Resistance Screening, and Plasmid Purification Based on Single-Plasmid Transformation of High-Copy Precursor Plasmid Backbone

This example tests the feasibility of the high-copy precursor plasmid backbone for the production of the plasmids without selectable markers in the present invention based on the pMF7-loxp-RFP precursor plasmid in Example 5. The specific implementation is the same as Example 8. The recombination of the precursor plasmid was achieved through induction. As shown in FIG. 35, the detection result of gel electrophoresis shows that the lane 2 is the precursor plasmid without induced recombination with a size of 3879 bp, the lane 3 is the plasmid without resistance gene generated by induced recombination for 1 h, where the band corresponding to the precursor plasmid is significantly weakened, and the size is correct. The pMF7-RFP plasmid without resistance selectable markers with a correct size of 1828 bp was obtained by subsequent resistance screening and purification. As shown in FIG. 36 (7 parallel plasmid products), the result of gel electrophoresis shows that the size is correct. The sequencing verification was performed on the purified pMF7-RFP plasmid. As shown in FIG. 37, the verification result indicates that the recombination is successful.

### Example 11: Induced Recombination, Resistance Screening, and Plasmid Purification Based on Double Plasmid System

In the double-plasmid recombination system, the recombinase expression system is loaded on the plasmid vector and transformed into the host strain together with the precursor plasmid. Multicopy plasmids can increase the expression amount of the recombinase and improve the recombination efficiency. The double-plasmid recombination system does not require strain modification, and is suitable for the preparation of plasmids without selectable markers in general commercial strains.

This example describes methods of induced recombination, resistance screening, and plasmid purification based on double-plasmid transformation by using the pSC101(ts)-araBAD-flp temperature-sensitive plasmid prepared in Example 2 and the pMF9-FRT-RFP plasmid prepared in Example 7. However, this example is not limited to the FLP-FRT recombination system. The specific operations are as follows:

11.1 Co-Transformation of pSC101(Ts)-araBAD-Flp Plasmid and pMF9-FRT-RFP Plasmid

1 μL of pSC101(ts)-araBAD-flp plasmid and 1 μL of pMF9-FRT-RFP plasmid were transformed with the JM108 competence as the host bacteria. The transformation steps are the same as Example 8.1.

### 11.2 the Addition of Arabinose Induces Flp-FRT Recombination

In this example, the arabinose was added into the JM108 pSC101(ts)-araBAD-flp pMF9-FRT-RFP culture system to induce the expression of the Flp enzyme, achieving the Flp-FRT recombination. The treatment of the control group is the same as that of the experimental group. The specific operations are as follows:
1) 3-4 JM108 pSC101(ts)-araBAD-flp pMF9-FRT-RFP colonies were selected to be inoculated in 4 mL of liquid culture medium of LB+Amp (100 μg/mL)+Kan (30 μg/mL)+1% glucose for culture at 30° C. and 220 rpm overnight, and the strains were enriched.
2) The strains were collected to be washed twice with antibiotic-free LB and then resuspended with an induction solution (induction solution: LB+2% arabinose). The induced recombination was carried out at 30° C. and 220 rpm for 2-8 h.
3) After the recombination time was reached, the recombined strains were transferred into a 4 mL antibiotic-free LB single tube by 1‰, the loss of pSC101(ts)-araBAD-flp was induced at 37° C., and then the strains were cultured at 220 rpm overnight.
4) antibiotic-free LB solid plate was streaked with 5 μL of bacterial solution from each of 3 test tubes cultured overnight to culture in an incubator at 37° C., and plasmids were extracted from the remaining bacterial solution for verification. The digestion system is shown in Table 27. AhdI was commercially available from New England Biotechnology Co., Ltd. under an article number of 10079968. Agarose gel electrophoresis was carried out (as shown in FIG. 38). The size of the precursor plasmid pMF9-FRT-RFP is 4309 bp. The recombined plasmid without markers is 2025 bp. The electrophoresis result shows that there are significant bands of the plasmid without markers after induction for 2-8 h (the plasmid lanes 2-4 and the plasmid digestion lanes 2-4), and there is significant loss of the pSC101(ts)-araBAD-flp plasmid after induction at 37° C. overnight (the plasmid lanes 5-7 and the plasmid digestion lanes 5-7).

TABLE 27
Digestion system

| System | Volume |
|---|---|
| Ahd I (10 U/μL) | 0.2 μL |
| 10 × cutsmart ® buffer (cat# B7204S) | 1 μL |
| Recombination product (100 ng/μL) | 1 μL |
| H₂O | 7.8 μL |

### 11.3 Kan and Amp Resistance Reverse Screening and Purification to Obtain Marker-Free Plasmid

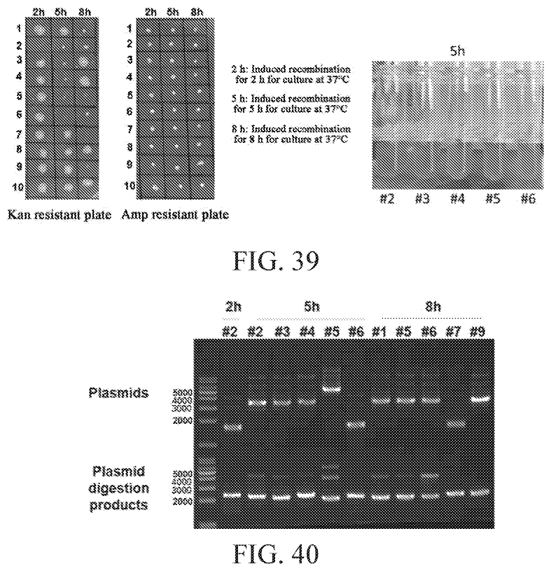

1) 10 single colonies were selected from each of the three antibiotic-free plates. The LB+Amp plate and the LB+Kan plate were dotted with each colony to culture in an incubator at 37° C. In addition, each colony was also inoculated in the LB antibiotic-free liquid culture medium to culture at 37° C. and 220 rpm for resistance screening. According to the screening result, the precursor plasmid and Kan resistance gene were removed from 10-50% of the single colonies after recombination, and all pSC101-araBAD-flp(ts) was lost after 37° C. induction, to obtain the strain without resistance (as shown in FIG. 39, the bacterial solution sample of the 5 h group is used as an example).
2) Colonies that had no colony grown on the LB+Amp plate and the LB+Kan plate and had colony grown in the LB antibiotic-free liquid culture medium were selected, and plasmids were extracted after preservation for digestion verification. The digestion system is shown in Table 20. Agarose gel electrophoresis was carried out. It has been verified that the plasmids and digestion products of 2h #2, 5h #6, and 8h #7 have correct sizes, which are plasmid products without markers (as shown in FIG. 40).

### Example 12: Induced Recombination, Resistance Screening, and Plasmid Purification Based on Double-Plasmid Transformation of R6Kγ Replicon

This example describes methods of induced recombination, resistance screening, and plasmid purification based on double-plasmid transformation by using the pSC101(ts)-amBAD-cre plasmid prepared in Example 1(4) and the pMF5-loxp-RFP precursor plasmid prepared in Example 6.

12.1 Co-Transformation of pSC101(Ts)-araBAD-Cre Plasmid and pMF5-Loxp-RFP Precursor Plasmid

The pir2 competence provided by Nanjing GenScript Biotechnology Co., Ltd. was selected for the host bacteria. 1 μL of pSC101(ts)-araBAD-cre plasmid and 1 μL of pMF5-loxp-RFP precursor plasmid were transformed. The transformation steps are the same as Example 8.1.

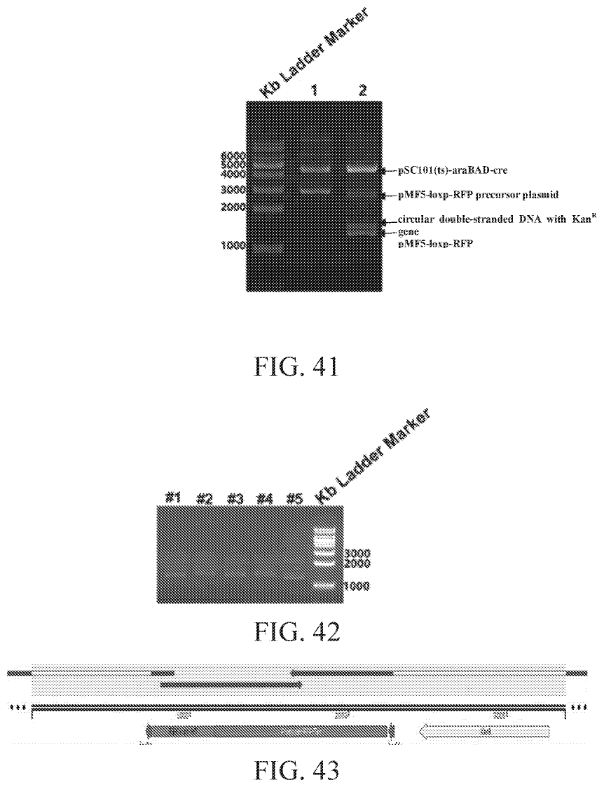

12.2 The specific implementations of recombination, screening, and purification are the same as Examples 8.2 and 8.3. The recombination of the precursor plasmid was achieved through induction. As shown in FIG. 41, the result shows that the lane 2 is a control of the plasmid without induced recombination containing two bands of the pSC101(ts)-araBAD-cre plasmid and pMF5-loxp-RFP precursor plasmid with sizes of 5867 bp and 3369 bp respectively. The lane 3 is bands of mixed plasmids obtained by induced recombination, and the bands from top to bottom are pSC101(ts)-araBAD-cre, pMF5-loxp-RFP precursor plasmid, circular double-stranded DNA carrying Kan^{R} gene, and pMF5-loxp-RFP. The band sizes are all correct. The pMF5-loxp-RFP plasmid without resistance selectable markers with a correct size of 1533 bp was obtained by subsequent resistance screening and purification. As shown in FIG. 42, the result shows that the size is correct. The sequencing verification was performed on the purified pMF5-loxp-RFP plasmid. As shown in FIG. 43, the verification result indicates that the recombination is successful.

### Example 13: Large-Scale Extraction of Marker-Free Plasmid

In this example, host E. coli cells carrying the pMF9-5.5 kb, pMF65-RFP, and pMF7-RFP plasmids with correct digestion and good plasmid supercoil bands in Examples 8, 9, and 10 were sent to Nanjing GenScript Biotechnology Co., Ltd. for large-scale plasmid preparation, referring to https://www.genscript.com.cn/industrial-grade-plasmid.html.

### QC Detection Result:

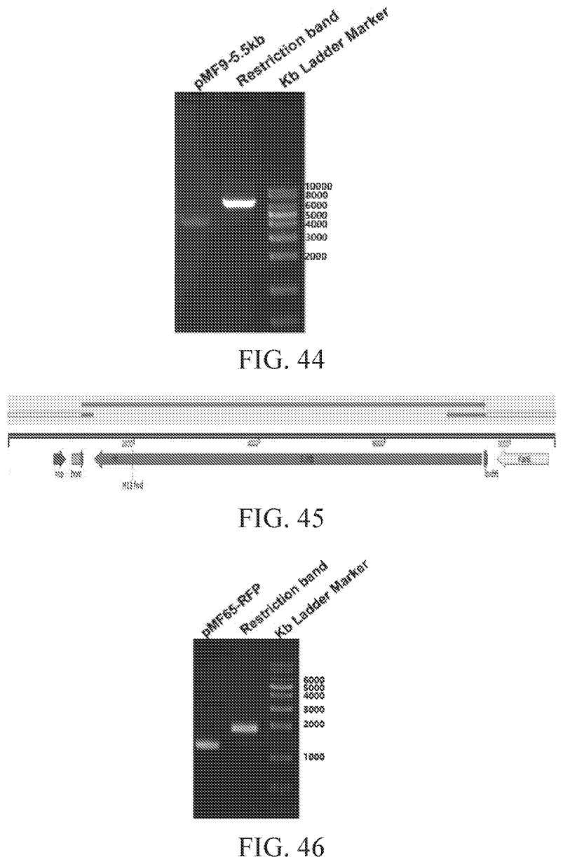

pMF9-5.5kb plasmid: 0.5-1.2 mg/L. Digestion verification was performed on 300 ng of the plasmids. The size of the plasmid is 6450 bp. The gel electrophoresis detection result shows that the band size is correct (as shown in FIG. 44). Analyzed by Tanon GIS, the proportion of monomeric supercoiled plasmid bands is greater than 90%. The sanger sequencing was performed on the plasmids, and the sequencing was completed by Nanjing GenScript Biotechnology Co., Ltd. The sequencing result shows that the sequence is correct, and there is no resistance gene sequence (as shown in FIG. 45).

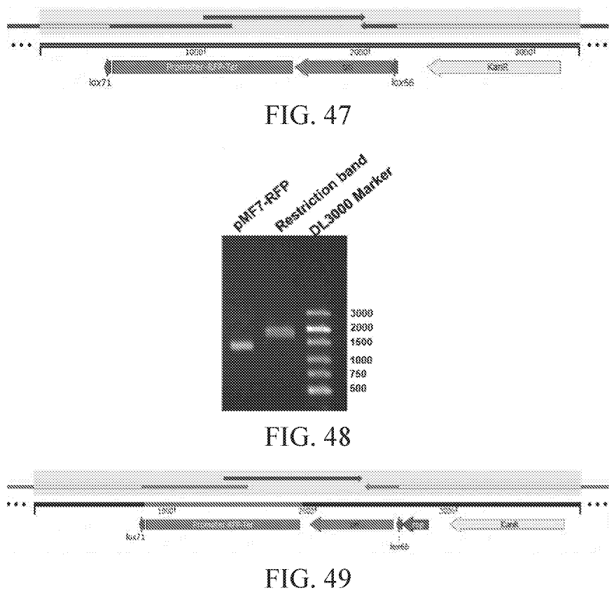

pMF65-RFP plasmid: 0.3-1 mg/L. Digestion verification was performed on 300 ng of the plasmids. The gel electrophoresis detection result shows that the size of the plasmid is 1743 bp, and the band size is correct (as shown in FIG. 46). Analyzed by Tanon GIS, the proportion of monomeric supercoiled plasmid bands is greater than 90%. The sanger sequencing was performed on the plasmids, and the sequencing was completed by Nanjing GenScript Biotechnology Co., Ltd. The sequencing result shows that the sequence is correct, and there is no resistance gene sequence (as shown in FIG. 47). After the NGS analysis on the plasmids, the content of the target daughter plasmid is greater than 98%, and the content of the precursor plasmid is less than 0.02%.

pMF7-RFP plasmid: 3.24.1 mg/L. Digestion verification was performed on 300 ng of the plasmids. The gel electrophoresis detection result shows that the size of the plasmid is 1828 bp, and the band size is correct (as shown in FIG. 48). Analyzed by Tanon GIS, the proportion of monomeric supercoiled plasmid bands is greater than 90%. The sanger sequencing was performed on the plasmids, and the sequencing was completed by Nanjing GenScript Biotechnology Co., Ltd. The sequencing result shows that the sequence is correct, and there is no resistance gene sequence (as shown in FIG. 49). After the NGS analysis on the plasmids, the content of the target daughter plasmid is greater than 98%, and the content of the precursor plasmid is less than 0.02%.

TABLE 28
Plasmid vector information in each example

| | | | Length of daughter | Copy |
|---|---|---|---|---|
| Precursor | | | plasmid backbone | number of |
| plasmid | Recombination | Replication | (without target | daughter |
| name | system | element | gene/MCS) | plasmid |
| pMF9-loxp | Cre-loxp | pMB1 ori | 864 bp | ~15-20 |
| (SEQ ID NO: 8) | | derivative | | |
| | | (SEQ ID NO: 43) | | |
| pMF65-loxp | Cre-loxp | pMB1 ori | 623 bp | ~15-20 |
| (SEQ ID NO: 34) | | (SEQ ID NO: 44) | | |
| pMF7-loxp | Cre-loxp | pUC ori | 708 bp | ~500-700 |
| (SEQ ID NO: 41) | | (SEQ ID NO: 45) | | |
| pMF5-loxp | Cre-loxp | R6Kγ ori | 429 bp | ~15-20 |
| (SEQ ID NO: 42) | | (SEQ ID NO: 46) | | |
| pMF9-FRT | FLP-FRT | pMB1 ori | 878 bp | ~15-20 |
| (SEQ ID NO: 47) | | derivative | | |
| | | (SEQ ID NO: 43) | | |

The implementations of the present invention are not limited to those described in the above examples. Without departing from the spirit and scope of the present invention, those of ordinary skill in the art can make various modifications and improvements to the present invention in form and details, and these are deemed to fall within the protection scope of the present invention.

Some nucleic acid sequence information mentioned in this specification is as follows:

| Gene name | Sequence |
|---|---|
| cre gene | caccatcaccatcaccatATGTCCAATTTACTGAC |
| | CGTACACCAAAATTTGCCTGCATTACCGGTCGATG |
| | CAACGAGTGATGAGGTTCGCAAGAACCTGATGGAC |
| | ATGTTCAGGGATCGCCAGGCGTTTTCTGAGCATAC |
| | CTGGAAAATGCTTCTGTCCGTTTGCCGGTCGTGGG |
| | CGGCATGGTGCAAGTTGAATAACCGGAAATGGTTT |
| | CCCGCAGAACCTGAAGATGTTCGCGATTATCTTCT |
| | ATATCTTCAGGCGCGCGGTCTGGCAGTAAAAACTA |
| | TCCAGCAACATTTGGGCCAGCTAAACATGCTTCAT |
| | CGTCGGTCCGGGCTGCCACGACCAAGTGACAGCAA |
| | TGCTGTTTCACTGGTTATGCGGCGGATCCGAAAAG |
| | AAAACGTTGATGCCGGTGAACGTGCAAAACAGGCT |
| | CTAGCGTTCGAACGCACTGATTTCGACCAGGTTCG |
| | TTCACTCATGGAAAATAGCGATCGCTGCCAGGATA |
| | TACGTAATCTGGCATTTCTGGGGATTGCTTATAAC |
| | ACCCTGTTACGTATAGCCGAAATTGCCAGGATCAG |
| | GGTTAAAGATATCTCACGTACTGACGGTGGGAGAA |
| | TGTTAATCCATATTGGCAGAACGAAAACGCTGGTT |
| | AGCACCGCAGGTGTAGAGAAGGCACTTAGCCTGGG |
| | GGTAACTAAACTGGTCGAGCGATGGATTTCCGTCT |
| | CTGGTGTAGCTGATGATCCGAATAACTACCTGTTT |
| | TGCCGGGTCAGAAAAAATGGTGTTGCCGCGCCATC |
| | TGCCACCAGCCAGCTATCAACTCGCGCCCTGGAAG |
| | GGATTTTTGAAGCAACTCATCGATTGATTTACGGC |
| | GCTAAGGATGACTCTGGTCAGAGATACCTGGCCTG |
| | GTCTGGACACAGTGCCCGTGTCGGAGCCGCGCGAG |
| | ATATGGCCCGCGCTGGAGTTTCAATACCGGAGATC |
| | ATGCAAGCTGGTGGCTGGACCAATGTAAATATTGT |
| | CATGAACTATATCCGTAACCTGGATAGTGAAACAG |
| | GGGCAATGGTGCGCCTGCTGGAAGATGGCGATTAA |
| | (SEQ ID NO: 1) |
| pMF9-loxp | TTCTCATGTTTGACAGCTTATCATCGATAAGCTTT |
| precursor | AATGCGGTAGTTTATCACAGTTAAATTGCTAACGC |
| empty | AGTCAGGCACCGTGTATGGAAGCCGGCGGCACCTC |
| vector | GCTAACGGATTCACCACTCCAAGAATTGGAGCCAA |
| plasmid | TCAATTCTTGCGGAGAACTGTGAATGCGCAAACCA |
| | ACCCTTGGCAGAACATATCCATCGCGTCCGCCATC |
| | TCCAGCAGCCGCACGCGGCGCATCTCGGGCAGCGT |
| | TGGGTCCTGGCCACGGGTGCGCATGATCGTGCTCC |
| | TGTCGTTGAGGACCCGGCTAGGCTGGCGGGGTTGC |
| | CTTACTGGTTAGCAGAATGAATCACCGATACGCGA |
| | GCGAACGTGAAGCGACTGCTGCTGCAAAACGTCTG |
| | CGACCTGAGCAACAACATGAATGGTCTTCGGTTTC |
| | CGTGTTTCGTAAAGTCTGGAAACGCGGAAGTCAGC |
| | GCCCTGCACCATTATGTTCCGGATCTGCATCGCAG |
| | GATGCTGCTGGCTACCCTGTGGAACACCTACATCT |
| | GTATTAACGAAGCGCTGGCATTGACCCTGAGTGAT |
| | TTTTCTCTGGTCCCGCCGCATCCATACCGCCAGTT |
| | GTTTACCCTCACAACGTTCCAGTAACCGGGCATGT |
| | TCATCATCAGTAACCCGTATCGTGAGCATCCTCTC |
| | TCGTTTCATCGGTATCATTACCCCCATGAACAGAA |
| | ATCCCCCTTACACGGAGGCATCAGTGACCAAACAG |
| | GAAAAAACCGCCCTTAACATGGCCCGCTTTATCAG |
| | AAGCCAGACATTAACGCTTCTGGAGAAACTCAACG |
| | AGCTGGACGCGGATGAACAGGCAGACATCTGTGAA |
| | TCGCTTCACGACCACGCTGATGAGCTTTACCGCAG |
| | CTGCCTCGCGCGTTTCGGTGATGACGGTGAAAACC |
| | TCTGACACATGCAGCTCCCGGAGACGGTCACAGCT |
| | TGTCTGTAAGCGGATGCCGGGAGCAGACAAGCCCG |
| | TCAGGGCGCGTCAGCGGGTGTTGGCGGGTGTCGGG |
| | GCGCAGCCATGACCCAGTCACGTAGCGATAGCGGA |
| | GTGTATACTGGCTTAACTATGCGGCATCAGAGCAG |
| | ATTGTACTGAGAGTGCACCATATGCGGTGTGAAAT |
| | ACCGCACAGATGCGTAAGGAGAAAATACCGCATCA |
| | GGataacttcgtataatgtatgctatacgaacggt |
| | aCGCTCTTCCGCTTCCTCGCTCACTGACTCGCTGC |
| | GCTCGGTCGTTCGGCTGCGGCGAGCGGTATCAGCT |
| | CACTCAAAGGCGGTAATACGGTTATCCACAGAATC |
| | AGGGGATAACGCAGGAAAGAACATGTGAGCAAAAG |
| | GCCAGCAAAAGGCCAGGAACCGTAAAAAGGCCGCG |
| | TTGCTGGCGTTTTTCCATAGGCTCCGCCCCCCTGA |
| | CGAGCATCACAAAAATCGACGCTCAAGTCAGAGGT |
| | GGCGAAACCCGACAGGACTATAAAGATACCAGGCG |
| | TTTCCCCCTGGAAGCTCCCTCGTGCGCTCTCCTGT |
| | TCCGACCCTGCCGCTTACCGGATACCTGTCCGCCT |
| | TTCTCCCTTCGGGAAGCGTGGCGCTTTCTCATAGC |
| | TCACGCTGTAGGTATCTCAGTTCGGTGTAGGTCGT |
| | TCGCTCCAAGCTGGGCTGTGTGCACGAACCCCCCG |
| | TTCAGCCCGACCGCTGCGCCTTATCCGGTAACTAT |
| | CGTCTTGAGTCCAACCCGGTAAGACACGACTTATC |
| | GCCACTGGCAGCAGCCACTGGTAACAGGATTAGCA |
| | GAGCGAGGTATGTAGGCGGTGCTACAGAGTTCTTG |
| | AAGTGGTGGCCTAACTACGGCTACACTAGAAGGAC |
| | AGTATTTGGTATCTGCGCTCTGCTGAAGCCAGTTA |
| | CCTTCGGAAAAAGAGTTGGTAGCTCTTGATCCGGC |
| | AAACAAACCACCGCTGGTAGCGGTGGTTTTTTTGT |
| | TTGCAAGCAGCAGATTACGCGCAGAAAAAAAGGAT |
| | CTCAAGAAGATCCTTGTAAAACGACGGCCAGTGAA |
| | TTCGAGCTCGGTACCCGGGGATCCTCTAGAGTCGA |
| | CCTGCAGGCATGCAAGCTTGGCGTAATCATGGTCA |
| | TAGCTGTTTCCTGtaccgttcgtataatgtatgct |
| | atacgaagttatTGATCTTTTCTACGGGGTCTGAC |
| | GCTCAGTGGAACGAAAACTCACGTTAAGGGATTTT |
| | GGTCATGAGATTATCAAAAAGGATCTTCACCTAGA |
| | TCCTTTTAAATTAAAAATGAAGTTTTAAATCAATC |
| | TAAAGTATATATGAGTAAACTTGGTCTGACAGtta |
| | gaaaaactcatcgagcatcaaatgaaactgcaatt |
| | tattcatatcaggattatcaataccatatttttga |
| | aaaagccgtttctgtaatgaaggagaaaactcacc |
| | gaggcagttccataggatggcaagatcctggtatc |
| | ggtctgcgattccgactcgtccaacatcaatacaa |
| | cctattaatttcccctcgtcaaaaataaggttatc |
| | aagtgagaaatcaccatgagtgacgactgaatccg |
| | gtgagaatggcaaaagtttatgcatttctttccag |
| | acttgttcaacaggccagccattacgctcgtcatc |
| | aaaatcactcgcatcaaccaaaccgttattcattc |
| | gtgattgcgcctgagcgagacgaaatacgcgatcg |
| | ctgttaaaaggacaattacaaacaggaatcgaatg |
| | caaccggcgcaggaacactgccagcgcatcaacaa |
| | tattttcacctgaatcaggatattcttctaatacc |
| | tggaatgctgttttcccggggatcgcagtggtgag |
| | taaccatgcatcatcaggagtacggataaaatgct |
| | tgatggtcggaagaggcataaattccgtcagccag |
| | tttagtctgaccatctcatctgtaacatcattggc |
| | aacgctacctttgccatgtttcagaaacaactctg |
| | gcgcatcgggcttcccatacaatcgatagattgtc |
| | gcacctgattgcccgacattatcgcgagcccattt |
| | atacccatataaatcagcatccatgttggaattta |
| | atcgcggcctagagcaagacgtttcccgttgaata |
| | tggctcatCACATTTCCCCGAAAAGTGCCACCTGA |
| | CGTCTAAGAAACCATTATTATCATGACATTAACCT |
| | ATAAAAATAGGCGTATCACGAGGCCCTTTCGTCTT |
| | CAAGAA |
| | (SEQ ID NO: 8) |
| gRNAII | GATGAACGTGCCGGAAAGCG |
| | (SEQ ID NO: 9) |
| JM108- | GGGGGTTGTTCTTCGAACGCCTGAATGTATCGGTA |
| araBAD- | CAGAAACTGGAAAACGCCATCGAGTTTTATGGCGC |
| cre(Amp) | GATTTCGCATAACAAACTGCACGGCCTGTCAGTGC |
| L-arm | AGGTTGAAACCAATCACGTCAAATTACCGGCGGTG |
| | GTGGACGGCTTTGCCAGCGAAGGGATCATCCAGTT |
| | CTTTTTCGAAGAAACGCAAGACGAGAATGGCTTTA |
| | ATATCTACATTCTCGACGAAAGCAACCGGGTTGAG |
| | GTATATCACCACTGCGAAGGCAGCAAAGAGGAGCT |
| | GGTACGTGACGTCAGTCGCTTCTACTCGTCATCGC |
| | ATGACCGTTTTACCTACGGCTCAAGCTTCATCAAC |
| | TTCAACCTGCCGCAGTTCTATCAGATTGTGAAGGT |
| | TGATGGTCGTGAACAGGTGATTCCGTTCCGCACAA |
| | AATCTATCGGTAACATGCCGCCTGCCAATCAGGAT |
| | CACGATACGCCGCTATTACAGCAATATTTTTCGTG |
| | ATGAACGTG |
| | (SEQ ID NO: 10) |
| JM108- | ATGCAATCTTAGCGGAAACTGACTGTTTCACCCGC |
| araBAD- | CTGCTGCGTCGCCGCCTGTTCCAGCAAATCCCAGA |
| cre(Amp)R- | AGGTTTCGCCGCTGCGATCACAAATCCACTCATCG |
| arm | CCTTTCAGGTCAAAATGGTAGCCGCCCTGTTTGGT |
| | TGCCAGCCATACCTGGTGCAGCGGCTCCTGGCGGT |
| | TGATAATGATTTTGCTGCCATTCTCAAAGGTAATG |
| | GTCAGTACGCCGCCGTTGATTTCGCAGTCGATATC |
| | GCTGTCGCCATCCCAGTCGTCCAGGCGTTCTTCAA |
| | TGGTCAGCCAGAGTTGATCAGCCAGGCGATGAAAT |
| | TCACTGTCGTTCATTGTTGTATCCTGTTTTTAAGT |
| | GATGGCGGCAGTATAGCGGCATGGGGTCAGGGCTT |
| | CAAAGTTTGCACCTCTGCGGCTGCGTTCCGGCACG |
| | ATTCATCCGTCACCGGAATAATGATGTCTCTGTGT |
| | AGCGAAAGATTTGTCTCTTCATTAGGGCGCAGTTA |
| | CACCAC(SEQ ID NO: 11) |
| Cre specific | TTATGACAACTTGACGGCTACATCATTCACTTTTT |
| recombinase | CTTCACAACCGGCACGGAACTCGCTCGGGCTGGCC |
| araBAD-cre | CCGGTGCATTTTTTAAATACCCGCGAGAAATAGAG |
| inducible | TTGATCGTCAAAACCAACATTGCGACCGACGGTGG |
| expression | CGATAGGCATCCGGGTGGTGCTCAAAAGCAGCTTC |
| system | GCCTGGCTGATACGTTGGTCCTCGCGCCAGCTTAA |
| | GACGCTAATCCCTAACTGCTGGCGGAAAAGATGTG |
| | ACAGACGCGACGGCGACAAGCAAACATGCTGTGCG |
| | ACGCTGGCGATATCAAAATTGCTGTCTGCCAGGTG |
| | ATCGCTGATGTACTGACAAGCCTCGCGTACCCGAT |
| | TATCCATCGGTGGATGGAGCGACTCGTTAATCGCT |
| | TCCATGCGCCGCAGTAACAATTGCTCAAGCAGATT |
| | TATCGCCAGCAGCTCCGAATAGCGCCCTTCCCCTT |
| | GCCCGGCGTTAATGATTTGCCCAAACAGGTCGCTG |
| | AAATGCGGCTGGTGCGCTTCATCCGGGCGAAAGAA |
| | CCCCGTATTGGCAAATATTGACGGCCAGTTAAGCC |
| | ATTCATGCCAGTAGGCGCGCGGACGAAAGTAAACC |
| | CACTGGTGATACCATTCGCGAGCCTCCGGATGACG |
| | ACCGTAGTGATGAATCTCTCCTGGCGGGAACAGCA |
| | AAATATCACCCGGTCGGCAAACAAATTCTCGTCCC |
| | TGATTTTTCACCACCCCCTGACCGCGAATGGTGAG |
| | ATTGAGAATATAACCTTTCATTCCCAGCGGTCGGT |
| | CGATAAAAAAATCGAGATAACCGTTGGCCTCAATC |
| | GGCGTTAAACCCGCCACCAGATGGGCATTAAACGA |
| | GTATCCCGGCAGCAGGGGATCATTTTGCGCTTCAG |
| | CCATACTTTTCATACTCCCGCCATTCAGAGAAGAA |
| | ACCAATTGTCCATATTGCATCAGACATTGCCGTCA |
| | CTGCGTCTTTTACTGGCTCTTCTCGCTAACCAAAC |
| | CGGTAACCCCGCTTATTAAAAGCATTCTGTAACAA |
| | AGCGGGACCAAAGCCATGACAAAAACGCGTAACAA |
| | AAGTGTCTATAATCACGGCAGAAAAGTCCACATTG |
| | ATTATTTGCACGGCGTCACACTTTGCTATGCCATA |
| | GCATTTTTATCCATAAGATTAGOGGATCCTACCTG |
| | ACGCTTTTTATCGCAACTCTCTACTGTTTCTCCAT |
| | ACCCGTTTTTTTGGGATGcaccatcaccatcacca |
| | tATGTCCAATTTACTGACCGTACACCAAAATTTGC |
| | CTGCATTACCGGTCGATGCAACGAGTGATGAGGTT |
| | CGCAAGAACCTGATGGACATGTTCAGGGATCGCCA |
| | GGCGTTTTCTGAGCATACCTGGAAAATGCTTCTGT |
| | CCGTTTGCCGGTCGTGGGCGGCATGGTGCAAGTTG |
| | AATAACCGGAAATGGTTTCCCGCAGAACCTGAAGA |
| | TGTTCGCGATTATCTTCTATATCTTCAGGCGCGCG |
| | GTCTGGCAGTAAAAACTATCCAGCAACATTTGGGC |
| | CAGCTAAACATGCTTCATCGTCGGTCCGGGCTGCC |
| | ACGACCAAGTGACAGCAATGCTGTTTCACTGGTTA |
| | TGCGGCGGATCCGAAAAGAAAACGTTGATGCCGGT |
| | GAACGTGCAAAACAGGCTCTAGCGTTCGAACGCAC |
| | TGATTTCGACCAGGTTCGTTCACTCATGGAAAATA |
| | GCGATCGCTGCCAGGATATACGTAATCTGGCATTT |
| | CTGGGGATTGCTTATAACACCCTGTTACGTATAGC |
| | CGAAATTGCCAGGATCAGGGTTAAAGATATCTCAC |
| | GTACTGACGGTGGGAGAATGTTAATCCATATTGGC |
| | AGAACGAAAACGCTGGTTAGCACCGCAGGTGTAGA |
| | GAAGGCACTTAGCCTGGGGGTAACTAAACTGGTCG |
| | AGCGATGGATTTCCGTCTCTGGTGTAGCTGATGAT |
| | CCGAATAACTACCTGTTTTGCCGGGTCAGAAAAAA |
| | TGGTGTTGCCGCGCCATCTGCCACCAGCCAGCTAT |
| | CAACTCGCGCCCTGGAAGGGATTTTTGAAGCAACT |
| | CATCGATTGATTTACGGCGCTAAGGATGACTCTGG |
| | TCAGAGATACCTGGCCTGGTCTGGACACAGTGCCC |
| | GTGTCGGAGCCGCGCGAGATATGGCCCGCGCTGGA |
| | GTTTCAATACCGGAGATCATGCAAGCTGGTGGCTG |
| | GACCAATGTAAATATTGTCATGAACTATATCCGTA |
| | ACCTGGATAGTGAAACAGGGGCAATGGTGCGCCTG |
| | CTGGAAGATGGCGATTAA |
| | (SEQ ID NO: 22) |
| pSC101 | TATGGACAGTTTTCCCTTTGATATCTAACGGTGAA |
| ori(ts) | CAGTTGTTCTACTTTTGTTTGTTAGTCTTGATGCT |
| fragment | TCACTGATAGATACAAGAGCCATAAGAACCTCAGA |
| | TCCTTCCGTATTTAGCCAGTATGTTCTCTAGTGTG |
| | GTTCGTTGTTTTTGCGTGAGCCATGAGAACGAACC |
| | ATTGAGATCATGCTTACTTTGCATGTCACTCAAAA |
| | ATTTTGCCTCAAAACTGGTGAGCTGAATTTTTGCA |
| | GTTAAAGCATCGTGTAGTGTTTTTCTTAGTCCGTT |
| | ACGTAGGTAGGAATCTGATGTAATGGTTGTTGGTA |
| | TTTTGTCACCATTCATTTTTATCTGGTTGTTCTCA |
| | AGTTCGGTTACGAGATCCATTTGTCTATCTAGTTC |
| | AACTTGGAAAATCAACGTATCAGTCGGGCGGCCTC |
| | GCTTATCAACCACCAATTTCATATTGCTGTAAGTG |
| | TTTAAATCTTTACTTATTGGTTTCAAAACCCATTG |
| | GTTAAGCCTTTTAAACTCATGGTAGTTATTTTCAA |
| | GCATTAACATGAACTTAAATTCATCAAGGCTAATC |
| | TCTATATTTGCCTTGTGAGTTTTCTTTTGTGTTAG |
| | TTCTTTTAATAACCACTCATAAATCCTCATAGAGT |
| | ATTTGTTTTCAAAAGACTTAACATGTTCCAGATTA |
| | TATTTTATGAATTTTTTTAACTGGAAAAGATAAGG |
| | CAATATCTCTTCACTAAAAACTAATTCTAATTTTT |
| | CGCTTGAGAACTTGGCATAGTTTGTCCACTGGAAA |
| | ATCTCAAAGCCTTTAACCAAAGGATTCCTGATTTC |
| | CACAGTTCTCGTCATCAGCTCTCTGGTTGCTTTAG |
| | CTAATACACCATAAGCATTTTCCCTACTGATGTTC |
| | ATCATCTGAACGTATTGGTTATAAGTGAACGATAC |
| | CGTCCGTTCTTTCCTTGTAGGGTTTTCAATCGTGG |
| | GGTTGAGTAGTGCCACACAGCATAAAATTAGCTTG |
| | GTTTCATGCTCCGTTAAGTCATAGCGACTAATCGC |
| | TAGTTCATTTGCTTTGAAAACAACTAATTCAGACA |
| | TACATCTCAATTGGTCTAGGTGATTTTAATCACTA |
| | TACCAATTGAGATGGGCTAGTCAATGATAATTACT |
| | AGTCCTTTTCCTTTGAGTIGTGGGTATCTGTAAAT |
| | TCTGCTAGACCTTTGCTGGAAAACTTGTAAATTCT |
| | GCTAGACCCTCTGTAAATTCCGCTAGACCTITGTG |
| | TGTTTTTTTTGTTTATATTCAAGTGGTTATAATTT |
| | ATAGAATAAAGAAAGAATAAAAAAAGATAAAAAGA |
| | ATAGATCCCAGCCCTGTGTATAACTCACTACTTTA |
| | GTCAGTTCCGCAGTATTACAAAAGGATGTCGCAAA |
| | CGCTGTTTGCTCCTCTACAAAACAGACCTTAAAAC |
| | CCTAAAGGCTTAAGTAGCACCCTCGCAAGCTOGG |
| | (SEQ ID NO: 23) |
| Flp gene | TTATATGCGTCTATTTATGTAGGATGAAAGGTAGT |
| | CTAGTACCTCCTGTGATATTATCCCATTCCATGCG |
| | GGGTATCGTATGCTTCCTTCAGCACTACCCTTTAG |
| | CTGTTCTATATGCTGCCACTCCTCAATTGGATTAG |
| | TCTCATCCTTCAATGCTATCATTTCCTTTGATATT |
| | GGATCATATGCATAGTACCGAGAAACTAGTGCGAA |
| | GTAGTGATCAGGTATTGCTGTTATCTGATGAGTAT |
| | ACGTTGTCCTGGCCACGGCAGAAGCACGCTTATCG |
| | CTCCAATTTCCCACAACATTAGTCAACTCCGTTAG |
| | GCCCTTCATTGAAAGAAATGAGGTCATCAAATGTC |
| | TTCCAATGTGAGATTTTGGGCCATTTTTTATAGCA |
| | AAGATTGAATAAGGCGCATTTTTCTTCAAAGCTTT |
| | ATTGTACGATCTGACTAAGTTATCTTTTAATAATT |
| | GGTATTCCTGTTTATTGCTTGAAGAATTGCCGGTC |
| | CTATTTACTCGTTTTAGGACTGGTTCAGAATTCCT |
| | CAAAAATTCATCCAAATATACAAGTGGATCGATCC |
| | TACCCCTTGCGCTAAAGAAGTATATGTGCCTACTA |
| | ACGCTTGTCTTTGTCTCTGTCACTAAACACTGGAT |
| | TATTACTCCCAGATACTTATTTTGGACTAATTTAA |
| | ATGATTTCGGATCAACGTTCTTAATATCGCTGAAT |
| | CTTCCACAATTGATGAAAGTAGCTAGGAAGAGGAA |
| | TTGGTATAAAGTTTTTGTTTTTGTAAATCTCGAAG |
| | TATACTCAAACGAATTTAGTATTTTCTCAGTGATC |
| | TCCCAGATGCTTTCACCCTCACTTAGAAGTGCTTT |
| | AAGCATTTTTTTACTGTGGCTATTTCCCTTATCTG |
| | CTTCTTCCGATGATTCGAACTGTAATTGCAAACTA |
| | CTTACAATATCAGTGATATCAGATTGATGTTTTTG |
| | TCCATAGTAAGGAATAATTGTAAATTCCCAAGCAG |
| | GAATCAATTTCTTTAATGAGGCTTCCAGAATTGTT |
| | GCTTTTTGCGTCTTGTATTTAAACTGGAGTGATTT |
| | ATTGACAATATCGAAACTCAGCGAATTGCTTATGA |
| | TAGTATTATAGCTCATGAATGTGGCTCTCTTGATT |
| | GCTGTTCCGTTATGTGTAATCATCCAACATAAATA |
| | GGTTAGTTCAGCAGCACATAATGCTATTTTCTCAC |
| | CTGAAGGTCTTTCAAACCTTTCCACAAACTGACGA |
| | ACAAGCACCTTAGGTGGTGTTTTACATAATATACC |
| | AAATTGTGGCAT |
| | (SEQ ID NO: 24) |
| Lox71 | TACCGTTCGTATAATGTATGCTATACGAAGTTAT |
| | (SEQ ID NO: 25) |
| Lox66 | ATAACTTCGTATAATGTATGCTATACGAACGGTA |
| | (SEQ ID NO: 26) |
| FRT | GAAGTTCCTATACTTTCTAGAGAATAGGAACTTCG |
| | GAATAGGAACTTC |
| | (SEQ ID NO: 27) |
| rfp gene | GTACCCAATACGCAAACCGCCTCTCCCCGCGCGTT |
| | GGCCGATTCATTAATGCAGCTGGCACGACAGGTTT |
| | CCCGACTGGAAAGCGGGCAGTGAGCGCAACGCAAT |
| | TAATGTGAGTTAGCTCACTCATTAGGCACCCCAGG |
| | CTTTACACTTTATGCTTCCGGCTCGTATGTTGTGT |
| | GGAATTGTGAGCGGATAACAATTTCACACATACTA |
| | GAGAAAGAGGAGAAATACTAGATGGCTTCCTCCGA |
| | AGACGTTATCAAAGAGTTCATGCGTTTCAAAGTTC |
| | GTATGGAAGGTTCCGTTAACGGTCACGAGTTCGAA |
| | ATCGAAGGTGAAGGTGAAGGTCGTCCGTACGAAGG |
| | TACCCAGACCGCTAAACTGAAAGTTACCAAAGGTG |
| | GTCCGCTGCCGTTCGCTTGGGACATCCTGTCCCCG |
| | CAGTTCCAGTACGGTTCCAAAGCTTACGTTAAACA |
| | CCCGGCTGACATCCCGGACTACCTGAAACTGTCCT |
| | TCCCGGAAGGTTTCAAATGGGAACGTGTTATGAAC |
| | TTCGAAGACGGTGGTGTTGTTACCGTTACCCAGGA |
| | CTCCTCCCTGCAAGACGGTGAGTTCATCTACAAAG |
| | TTAAACTGCGTGGTACCAACTTCCCGTCCGACGGT |
| | CCGGTTATGCAGAAAAAAACCATGGGTTGGGAAGC |
| | TTCCACCGAACGTATGTACCCGGAAGACGGTGCTC |
| | TGAAAGGTGAAATCAAAATGCGTCTGAAACTGAAA |
| | GACGGTGGTCACTACGACGCTGAAGTTAAAACCAC |
| | CTACATGGCTAAAAAACCGGTTCAGCTGCCGGGTG |
| | CTTACAAAACCGACATCAAACTGGACATCACCTCC |
| | CACAACGAAGACTACACCATCGTTGAACAGTACGA |
| | ACGTGCTGAAGGTCGTCACTCCACCGGTGCTTAAT |
| | AACGCTGATAGTGCTAGTGTAGATCGCTACTAGAG |
| | CCAGGCATCAAATAAAACGAAAGGCTCAGTCGAAA |
| | GACTGGGCCTTTCGTTTTATCTGTTGTTTGTCGGT |
| | GAACGCTCTCTACTAGAGTCACACTGGCTCACCTT |
| | CGGGTGGGCCTTTCTGCGTTTATAGTCGACTCGAG |
| | CGGCC |
| | (SEQ ID NO: 28) |
| pUC57 | tcgcgcgtttcggtgatgacggtgaaaacctctga |
| (Kan^{R}) | cacatgcagctcccggagacggtcacagcttgtct |
| | gtaagcggatgccgggagcagacaagcccgtcagg |
| | gcgcgtcagcgggtgttggcgggtgtcggggctgg |
| | cttaactatgcggcatcagagcagattgtactgag |
| | agtgcaccatatgcggtgtgaaataccgcacagat |
| | gcgtaaggagaaaataccgcatcaggcgccattcg |
| | ccattcaggctgcgcaactgttgggaagggcgatc |
| | ggtgcgggcctcttcgctattacgccagctggcga |
| | aagggggatgtgctgcaaggcgattaagttgggta |
| | acgccagggttttcccagtcacgacgttgtaaaac |
| | gacggccagagaattcgagctcggtacctcgcgaa |
| | tacatctagatatcggatcccgggcccgtcgactg |
| | cagaggcctgcatgcaagcttggtgtaatcatggt |
| | catagctgtttcctgtgtgaaattgttatccgctc |
| | acaattccacacaacatacgagccggaagcataaa |
| | gtgtaaagcctggggtgcctaatgagtgagctaac |
| | tcacattaattgcgttgcgctcactgcccgctttc |
| | cagtcgggaaacctgtcgtgccagctgcattaatg |
| | aatcggccaacgcgcggggagaggcggtttgcgta |
| | ttgggcgctcttccgcttcctcgctcactgactcg |
| | ctgcgctcggtcgttcggctgcggcgagcggtatc |
| | agctcactcaaaggcggtaatacggttatccacag |
| | aatcaggggataacgcaggaaagaacatgtgagca |
| | aaaggccagcaaaaggccaggaaccgtaaaaaggc |
| | cgcgttgctggcgtttttccataggctccgccccc |
| | ctgacgagcatcacaaaaatcgacgctcaagtcag |
| | aggtggcgaaacccgacaggactataaagatacca |
| | ggcgtttccccctggaagctccctcgtgcgctctc |
| | ctgttccgaccctgccgcttaccggatacctgtcc |
| | gcctttctcccttcgggaagcgtggcgctttctca |
| | tagctcacgctgtaggtatctcagttcggtgtagg |
| | tcgttcgctccaagctgggctgtgtgcacgaaccc |
| | cccgttcagcccgaccgctgcgccttatccggtaa |
| | ctatcgtcttgagtccaacccggtaagacacgact |
| | tatcgccactggcagcagccactggtaacaggatt |
| | agcagagcgaggtatgtaggcggtgctacagagtt |
| | cttgaagtggtggcctaactacggctacactagaa |
| | gaacagtatttggtatctgcgctctgctgaagcca |
| | gttaccttcggaaaaagagttggtagctcttgatc |
| | cggcaaacaaaccaccgctggtagcggtggttttt |
| | tttttgcaagcagcagattacgcgcagaaaaaaag |
| | gatctcaagaagatcctttgatcttttctacgggg |
| | tctgacgctcagtggaacgaaaactcacgttaagg |
| | gattttggtcatgagattatcaaaaaggatcttca |
| | cctagatccttttaaattaaaaatgaagttttaaa |
| | tcaagcccaatctgaataatgttacaaccaattaa |
| | ccaattctgattagaaaaactcatcgagcatcaaa |
| | tgaaactgcaatttattcatatcaggattatcaat |
| | accatatttttgaaaaagccgtttctgtaatgaag |
| | gagaaaactcaccgaggcagttccataggatggca |
| | agatcctggtatcggtctgcgattccgactcgtcc |
| | aacatcaatacaacctattaatttcccctcgtcaa |
| | aaataaggttatcaagtgagaaatcaccatgagtg |
| | acgactgaatccggtgagaatggcaaaagtttatg |
| | catttctttccagacttgttcaacaggccagccat |
| | tacgctcgtcatcaaaatcactcgcatcaaccaaa |
| | ccgttattcattcgtgattgcgcctgagcgagacg |
| | aaatacgcgatcgctgttaaaaggacaattacaaa |
| | caggaatcgaatgcaaccggcgcaggaacactgcc |
| | agcgcatcaacaatattttcacctgaatcaggata |
| | ttcttctaatacctggaatgctgtttttccgggga |
| | tcgcagtggtgagtaaccatgcatcatcaggagta |
| | cggataaaatgcttgatggtcggaagaggcataaa |
| | ttccgtcagccagtttagtctgaccatctcatctg |
| | taacatcattggcaacgctacctttgccatgtttc |
| | agaaacaactctggcgcatcgggcttcccatacaa |
| | gcgatagattgtcgcacctgattgcccgacattat |
| | cgcgagcccatttatacccatataaatcagcatcc |
| | atgttggaatttaatcgcggcctcgacgtttcccg |
| | ttgaatatggctcataacaccccttgtattactgt |
| | ttatgtaagcagacagttttattgttcatgatgat |
| | atatttttatcttgtgcaatgtaacatcagagatt |
| | itgagacacgggccagagctgca |
| | (SEQ ID NO: 29) |
| pMF65-loxp | tcgcgcgtttcggtgatgacggtgaaaacctctga |
| precursor | cacatgcagctcccggagacggtcacagcttgtct |
| empty | gtaagcggatgccgggagcagacaagcccgtcagg |
| vector | gcgcgtcagcgggtgttggcgggtgtcggggctgg |
| plasmid | cttaactatgcggcatcagagcagattgtactgag |
| | agtgcaccatatgcggtgtgaaataccgcacagat |
| | gcgtaaggagaaaataccgcatcaggcgccattcg |
| | ccattcaggctgcgcaactgttgggaaggggatcg |
| | gtgcgggcctcttcgctattacgccagctggcgaa |
| | agggggatgtgctgcaaggcgattaagttgggtaa |
| | cgccagggttttcccagtcacgacgttgtaaaacg |
| | acggccagaataacttcgtataatgtatgctatac |
| | gaacggtagaattcggatcccgggcccgtcgactg |
| | cagtttccataggctccgcccccctgacgagcatc |
| | acaaaaatcgacgctcaagtcagaggtggcgaaac |
| | ccgacaggactataaagataccaggcgtttccccc |
| | tggaagctccctcgtgcgctctcctgttccgaccc |
| | tgccgcttaccggatacctgtccgcctttctccct |
| | tcgggaagcgtggcgctttctcatagctcacgctg |
| | taggtatctcagttcggtgtaggtgttcgctccaa |
| | gctgggctgtgtgcacgaaccccccgttcagcccg |
| | accgctgcgccttatccggtaactatcgtcttgag |
| | tccaacccggtaagacacgacttatcgccactggc |
| | agcagccactggtaacaggattagcagagcgaggt |
| | atgtaggcggtgctacagagttcttgaagtggtgg |
| | cctaactacggctacactagaagaacagtatttgg |
| | tatctgcgctctgctgaagccagttaccttcggaa |
| | aaagagttggtagctcttgatccggcaaacaaacc |
| | accgctggtagcggtggtttttttgtttgcaagca |
| | gcagattacgcgcagaaaaaaaggatctcaatacc |
| | gttcgtataatgtatgctatacgaagttatgaaga |
| | tcctttgatcttttctacggggtctgacgctcagt |
| | ggaacgaaaactcacgttaagggattttggtcatg |
| | agattatcaaaaaggatcttcacctagatcctttt |
| | aaattaaaaatgaagtittaaatcaagcccaatct |
| | gaataatgttacaaccaattaaccaattctgatta |
| | gaaaaactcatcgagcatcaaatgaaactgcaatt |
| | tattcatatcaggattatcaataccatatttttga |
| | aaaagccgtttctgtaatgaaggagaaaactcacc |
| | gaggcagttccataggatggcaagatcctggtatc |
| | ggtctgcgattccgactcgtccaacatcaatacaa |
| | cctattaatttcccctcgtcaaaaataaggttatc |
| | aagtgagaaatcaccatgagtgacgactgaatccg |
| | gtgagaatggcaaaagtttatgcatttctttccag |
| | acttgttcaacaggccagccattacgctcgtcatc |
| | aaaatcactcgcatcaaccaaaccgttattcattc |
| | gtgattgcgcctgagcgagacgaaatacgcgatcg |
| | ctgttaaaaggacaattacaaacaggaatcgaatg |
| | caaccggcgcaggaacactgccagcgcatcaacaa |
| | tattttcacctgaatcaggatattcttctaatacc |
| | tggaatgctgtttttccggggatcgcagtggtgag |
| | taaccatgcatcatcaggagtacggataaaatgct |
| | tgatggtcggaagaggcataaattccgtcagccag |
| | tttagtctgaccatctcatctgtaacatcattggc |
| | aacgctacctttgccatgtttcagaaacaactctg |
| | gcgcatcgggcttcccatacaagcgatagattgtc |
| | gcacctgattgcccgacattatcgcgagcccattt |
| | atacccatataaatcagcatccatgttggaattta |
| | atcgcggcctcgacgtttcccgttgaatatggctc |
| | ataacaccccttgtattactgtttatgtaagcaga |
| | cagttttattgttcatgatgatatatttttatctt |
| | gtgcaatgtaacatcagagattttgagacacgggc |
| | cagagctgca(SEQ ID NO: 34) |
| pMF7-loxp- | tcgcgcgtttcggtgatgacggtgaaaacctctga |
| RFP | cacatgcagctcccggagacggtcacagcttgtct |
| precursor | gtaagcggatgccgggagcagacaagcccgtcagg |
| plasmid | gcgcgtcagcgggtgttggcgggtgtcggggctgg |
| | cttaactatgcggcatcagagcagattgtactgag |
| | agtgcaccatatgcggtgtgaaataccgcacagat |
| | gcgtaaggagaaaataccgcatcaggcgccattcg |
| | ccattcaggctgcgcaactgttgggaagggcgatc |
| | ggtgcgggcctcttcgctattacgccagctggcga |
| | aagggggatgtgctgcaaggcgattaagttgggta |
| | acgccagggttttcccagtcacgacgttgtaaaac |
| | gacggccagagggtgtaatcatggtcatagctgtt |
| | tcctgtgtgaaattgttatccgctcacaattccac |
| | acaacatacgagccggaagcataaagtgtaaagcc |
| | tggggtgcctaatgagtgagctaactcacattaat |
| | tgcgttgcgctcactgcccgctttccagtcgggaa |
| | acctgtcgtgccagctgcattaatgaatcggccaa |
| | cgcgcggggagaggcggtttgcgtattgggcgctc |
| | ttccgcttcctcgctcactgactcgctgcgctcgg |
| | tcgttcggctgcggcgagcggtatcagctcactca |
| | aaggcggtaatacggttatccacagaatcagggga |
| | taacgcaggaaagaacataacttcgtataatgtat |
| | gctatacgaacggtagaattcggatcccGTACCCA |
| | ATACGCAAACCGCCTCTCCCCGCGCGTTGGCCGAT |
| | TCATTAATGCAGCTGGCACGACAGGTTTCCCGACT |
| | GGAAAGCGGGCAGTGAGCGCAACGCAATTAATGTG |
| | AGTTAGCTCACTCATTAGGCACCCCAGGCTTTACA |
| | CTTTATGCTTCCGGCTCGTATGTTGTGTGGAATTG |
| | TGAGCGGATAACAATTTCACACATACTAGAGAAAG |
| | AGGAGAAATACTAGATGGCTTCCTCCGAAGACGTT |
| | ATCAAAGAGTTCATGCGTTTCAAAGTTCGTATGGA |
| | AGGTTCCGTTAACGGTCACGAGTTCGAAATCGAAG |
| | GTGAAGGTGAAGGTCGTCCGTACGAAGGTACCCAG |
| | ACCGCTAAACTGAAAGTTACCAAAGGTGGTCCGCT |
| | GCCGTTCGCTTGGGACATCCTGTCCCCGCAGTTCC |
| | AGTACGGTTCCAAAGCTTACGTTAAACACCCGGCT |
| | GACATCCCGGACTACCTGAAACTGTCCTTCCCGGA |
| | AGGTTTCAAATGGGAACGTGTTATGAACTTCGAAG |
| | ACGGTGGTGTTGTTACCGTTACCCAGGACTCCTCC |
| | CTGCAAGACGGTGAGTTCATCTACAAAGTTAAACT |
| | GCGTGGTACCAACTTCCCGTCCGACGGTCCGGTTA |
| | TGCAGAAAAAAACCATGGGTTGGGAAGCTTCCACC |
| | GAACGTATGTACCCGGAAGACGGTGCTCTGAAAGG |
| | TGAAATCAAAATGCGTCTGAAACTGAAAGACGGTG |
| | GTCACTACGACGCTGAAGTTAAAACCACCTACATG |
| | GCTAAAAAACCGGTTCAGCTGCCGGGTGCTTACAA |
| | AACCGACATCAAACTGGACATCACCTCCCACAACG |
| | AAGACTACACCATCGTTGAACAGTACGAACGTGCT |
| | GAAGGTCGTCACTCCACCGGTGCTTAATAACGCTG |
| | ATAGTGCTAGTGTAGATCGCTACTAGAGCCAGGCA |
| | TCAAATAAAACGAAAGGCTCAGTCGAAAGACTGGG |
| | CCTTTCGTTTTATCTGTTGTTTGTCGGTGAACGCT |
| | CTCTACTAGAGTCACACTGGCTCACCTTCGGGTGG |
| | GCCTTTCTGCGTTTATAGTCGACTCGAGCGGCCgg |
| | gcccgtcgactgcagatgtgagcaaaaggccagca |
| | aaaggccaggaaccgtaaaaaggccgcgttgctgg |
| | cgtttttccataggctccgcccccctgacgagcat |
| | cacaaaaatcgacgctcaagtcagaggtggcgaaa |
| | cccgacaggactataaagataccaggcgtttcccc |
| | ctggaagctccctcgtgcgctctcctgttccgacc |
| | ctgccgcttaccggatacctgtccgcctttctccc |
| | ttcgggaagcgtggcgctttctcatagctcacgct |
| | gtaggtatctcagttcggtgtaggtcgttcgctcc |
| | aagctgggctgtgtgcacgaaccccccgttcagcc |
| | cgaccgctgcgccttatccggtaactatcgtcttg |
| | agtccaacccggtaagacacgacttatcgccactg |
| | gcagcagccactggtaacaggattagcagagcgag |
| | gtatgtaggcggtgctacagagttcttgaagtggt |
| | ggcctaactacggctacactagaagaacagtattt |
| | ggtatctgcgctctgctgaagccagttaccttcgg |
| | aaaaagagttggtagctcttgatccggcaaacaaa |
| | ccaccgctggtagcggtggtttttttgtttgcaag |
| | cagcagattacgcgcagaaaaaaaggatctcaaga |
| | agatcctttgatcttttctacgggtaccgttcgta |
| | taatgtatgctatacgaagttatTCAGAGGTTTTC |
| | ACCGTCATCACCGAAACGCGCGAGGCAGCTGCGGT |
| | AAAGCTCATCAGCGTGGTCGTGAAGCGATTCACAG |
| | ATGTCTGCCTGTTCATCCGCGTCCAGCTCGTTGAG |
| | TTTCTCCAGAAGCGTTAATGTCTGGCTTCTGATAA |
| | AGCGGGCCATGTTAAGGGCGGTTTTTTCCTGTTTG |
| | GTCACgtctgacgctcagtggaacgaaaactcacg |
| | ttaagggattttggtcatgagattatcaaaaagga |
| | tcttcacctagatccttttaaattaaaaatgaagt |
| | tttaaatcaagcccaatctgaataatgttacaacc |
| | aattaaccaattctgattagaaaaactcatcgagc |
| | atcaaatgaaactgcaatttattcatatcaggatt |
| | atcaataccatatttttgaaaaagccgtttctgta |
| | atgaaggagaaaactcaccgaggcagttccatagg |
| | atggcaagatcctggtatcggtctgcgattccgac |
| | tcgtccaacatcaatacaacctattaatttcccct |
| | cgtcaaaaataaggttatcaagtgagaaatcacca |
| | tgagtgacgactgaatccggtgagaatggcaaaag |
| | tttatgcatttctttccagacttgttcaacaggcc |
| | agccattacgctcgtcatcaaaatcactcgcatca |
| | accaaaccgttattcattcgtgattgcgcctgagc |
| | gagacgaaatacgcgatcgctgttaaaaggacaat |
| | tacaaacaggaatcgaatgcaaccggcgcaggaac |
| | actgccagcgcatcaacaatattttcacctgaatc |
| | aggatattcttctaatacctggaatgctgtttttc |
| | cggggatcgcagtggtgagtaaccatgcatcatca |
| | ggagtacggataaaatgcttgatggtcggaagagg |
| | cataaattccgtcagccagtttagtctgaccatct |
| | catctgtaacatcattggcaacgctacctttgcca |
| | tgtttcagaaacaactctggcgcatcgggcttccc |
| | atacaagcgatagattgtcgcacctgattgcccga |
| | cattatcgcgagcccatttatacccatataaatca |
| | gcatccatgttggaatttaatcgcggcctcgacgt |
| | ttcccgttgaatatggctcataacaccccttgtat |
| | tactgtttatgtaagcagacagttttattgttcat |
| | gatgatatatttttatcttgtgcaatgtaacatca |
| | gagattttgagacacgggccagagctgca |
| | (SEQ ID NO: 39) |
| pMF5-loxp- | TTCTCATGITTGACAGCTTATCATCGATAAGCTTT |
| RFP | AATGCGGTAGTTTATCACAGTTAAATTGCTAACGC |
| precursor | AGTCAGGCACCGTGTATGGAAGCCGGCGGCACCTC |
| plasmid | GCTAACGGATTCACCACTCCAAGAATTGGAGCCAA |
| | TCAATTCTTGCGGAGAACTGTGAATGCGCAAACCA |
| | ACCCTTGGCAGAACATATCCATCGCGTCCGCCATC |
| | TCCAGCAGCCGCACGCGGCGCATCTCGGGCAGCGT |
| | TGGGTCCTGGCCACGGGTGCGCATGATCGTGCTCC |
| | TGTCGTTGAGGACCCGGCTAGGCTGGCGGGGTTGC |
| | CTTACTGGTTAGCAGAATGAATCACCGATACGCGA |
| | GCGAACGTGAAGCGACTGCTGCTGCAAAACGTCTG |
| | CGACCTGAGCAACAACATGAATGGTCTTCGGTTTC |
| | CGTGTTTCGTAAAGTCTGGAAACGCGGAAGTCAGC |
| | GCCCTGCACCATTATGTTCCGGATCTGCATCGCAG |
| | GATGCTGCTGGCTACCCTGTGGAACACCTACATCT |
| | GTATTAACGAAGCGCTGGCATTGACCCTGAGTGAT |
| | TTTTCTCTGGTCCCGCCGCATCCATACCGCCAGTT |
| | GTTTACCCTCACAACGTTCCAGTAACCGGGCATGT |
| | TCATCATCAGTAACCCGTATCGTGAGCATCCTCTC |
| | TCGTTTCATCGGTATCATTACCCCCATGAACAGAA |
| | ATCCCCCTTACACGGAGGCATCAataacttcgtat |
| | aatgtatgctatacgaacggtaCTGCAGtgtcagc |
| | cgttaagtgttcctgtgtcactcaaaattgctttg |
| | agaggctctaagggcttctcagtgcgttacatccc |
| | tggcttgttgtccacaaccgttaaaccttaaaagc |
| | tttaaaagccttatatattcttttttttcttataa |
| | aacttaaaaccttagaggctatttaagttgctgat |
| | ttatattaattttattgttcaaacatgagagctta |
| | gtacgtgaaacatgagagcttagtacgttagccat |
| | gagagcttagtacgttagccatgagggtttagttc |
| | gttaaacatgagagcttagtacgttaaacatgaga |
| | gcttagtacgtgaaacatgagagcttagtacgtac |
| | tatcaacaggttgaactgctgatcttcagatcGGT |
| | ACCCAATACGCAAACCGCCTCTCCCCGCGCGTTGG |
| | CCGATTCATTAATGCAGCTGGCACGACAGGTTTCC |
| | CGACTGGAAAGCGGGCAGTGAGCGCAACGCAATTA |
| | ATGTGAGTTAGCTCACTCATTAGGCACCCCAGGCT |
| | TTACACTTTATGCTTCCGGCTCGTATGTTGTGTGG |
| | AATTGTGAGCGGATAACAATTTCACACATACTAGA |
| | GAAAGAGGAGAAATACTAGATGGCTTCCTCCGAAG |
| | ACGTTATCAAAGAGTTCATGCGTTTCAAAGTTCGT |
| | ATGGAAGGTTCCGTTAACGGTCACGAGTTCGAAAT |
| | CGAAGGTGAAGGTGAAGGTCGTCCGTACGAAGGTA |
| | CCCAGACCGCTAAACTGAAAGTTACCAAAGGTGGT |
| | CCGCTGCCGTTCGCTTGGGACATCCTGTCCCCGCA |
| | GTTCCAGTACGGTTCCAAAGCTTACGTTAAACACC |
| | CGGCTGACATCCCGGACTACCTGAAACTGTCCTTC |
| | CCGGAAGGTTTCAAATGGGAACGTGTTATGAACTT |
| | CGAAGACGGTGGTGTTGTTACCGTTACCCAGGACT |
| | CCTCCCTGCAAGACGGTGAGTTCATCTACAAAGTT |
| | AAACTGCGTGGTACCAACTTCCCGTCCGACGGTCC |
| | GGTTATGCAGAAAAAAACCATGGGTTGGGAAGCTT |
| | CCACCGAACGTATGTACCCGGAAGACGGTGCTCTG |
| | AAAGGTGAAATCAAAATGCGTCTGAAACTGAAAGA |
| | CGGTGGTCACTACGACGCTGAAGTTAAAACCACCT |
| | ACATGGCTAAAAAACCGGTTCAGCTGCCGGGTGCT |
| | TACAAAACCGACATCAAACTGGACATCACCTCCCA |
| | CAACGAAGACTACACCATCGTTGAACAGTACGAAC |
| | GTGCTGAAGGTCGTCACTCCACCGGTGCTTAATAA |
| | CGCTGATAGTGCTAGTGTAGATCGCTACTAGAGCC |
| | AGGCATCAAATAAAACGAAAGGCTCAGTCGAAAGA |
| | CTGGGCCTTTCGTTTTATCTGTTGTTTGTCGGTGA |
| | ACGCTCTCTACTAGAGTCACACTGGCTCACCTTCG |
| | GGTGGGCCTTTCTGCGTTTATAGTCGACTCGAGCG |
| | GCCAGTCGACGCATGCtaccgttcgtataatgtat |
| | gctatacgaagttatTGATCTTTTCTACGGGGTCT |
| | GACGCTCAGTGGAACGAAAACTCACGTTAAGGGAT |
| | TTTGGTCATGAGATTATCAAAAAGGATCTTCACCT |
| | AGATCCTTTTAAATTAAAAATGAAGTTTTAAATCA |
| | ATCTAAAGTATATATGAGTAAACTTGGTCTGACAG |
| | ttagaaaaactcatcgagcatcaaatgaaactgca |
| | atttattcatatcaggattatcaataccatatttt |
| | tgaaaaagccgtttctgtaatgaaggagaaaactc |
| | accgaggcagttccataggatggcaagatcctggt |
| | atcggtctgcgattccgactcgtccaacatcaata |
| | caacctattaatttcccctcgtcaaaaataaggtt |
| | atcaagtgagaaatcaccatgagtgacgactgaat |
| | ccggtgagaatggcaaaagtttatgcatttctttc |
| | cagacttgttcaacaggccagccattacgctcgtc |
| | atcaaaatcactcgcatcaaccaaaccgttattca |
| | ttcgtgattgcgcctgagcgagacgaaatacgcga |
| | tcgctgttaaaaggacaattacaaacaggaatcga |
| | atgcaaccggcgcaggaacactgccagcgcatcaa |
| | caatattttcacctgaatcaggatattcttctaat |
| | acctggaatgctgttttcccggggatcgcagtggt |
| | gagtaaccatgcatcatcaggagtacggataaaat |
| | gcttgatggtcggaagaggcataaattccgtcagc |
| | cagtttagtctgaccatctcatctgtaacatcatt |
| | ggcaacgctacctttgccatgtttcagaaacaact |
| | ctggcgcatcgggcttcccatacaatcgatagatt |
| | gtcgcacctgattgcccgacattatcgcgagccca |
| | tttatacccatataaatcagcatccatgttggaat |
| | itaatcgcggcctagagcaagacgtttcccgttga |
| | atatggctcatCACATTTCCCCGAAAAGTGCCACC |
| | TGACGTCTAAGAAACCATTATTATCATGACATTAA |
| | CCTATAAAAATAGGCGTATCACGAGGCCCTTTCGT |
| | CTTCAAGAA |
| | (SEQ ID NO: 40) |
| pMF7-loxp | tcgcgcgtttcggtgatgacggtgaaaacctctga |
| precursor | cacatgcagctcccggagacggtcacagcttgtct |
| empty | gtaagcggatgccgggagcagacaagcccgtcagg |
| vector | gcgcgtcagcgggtgttcgcgggtgtcggggctgg |
| plasmid | cttaactatgcggcatcagagcagattgtactgag |
| | agtgcaccatatgcggtgtgaaataccgcacagat |
| | gcgtaaggagaaaataccgcatcaggcgccattcg |
| | ccattcaggctgcgcaactgttgggaagggcgatc |
| | ggtgcgggcctcttcgctattacgccagctggcga |
| | aagggggatgtgctgcaaggcgattaagttgggta |
| | acgccagggttttcccagtcacgacgttgtaaaac |
| | gacggccagagggtgtaatcatggtcatagctgtt |
| | tcctgtgtgaaattgttatccgctcacaattccac |
| | acaacatacgagccggaagcataaagtgtaaagcc |
| | tggggtgcctaatgagtgagctaactcacattaat |
| | tgcgttgcgctcactgcccgctttccagtcgggaa |
| | acctgtcgtgccagctgcattaatgaatcggccaa |
| | cgcgcggggagaggcggtttgcgtattgggcgctc |
| | ttccgcttcctcgctcactgactcgctgcgctcgg |
| | tcgttcggctgcggcgagcggtatcagctcactca |
| | aaggcggtaatacggttatccacagaatcagggga |
| | taacgcaggaaagaacataacttcgtataatgtat |
| | gctatacgaacggtagaattcggatcccgggcccg |
| | tcgactgcagatgtgagcaaaaggccagcaaaagg |
| | ccaggaaccgtaaaaaggccgcgttgctggcgttt |
| | ttccataggctccgcccccctgacgagcatcacaa |
| | aaatcgacgctcaagtcagaggtggcgaaacccga |
| | caggactataaagataccaggcgtttccccctgga |
| | agctccctcgtgcgctctcctgttccgaccctgcc |
| | gcttaccggatacctgtccgcctttctcccttcgg |
| | gaagcgtggcgctttctcatagctcacgctgtagg |
| | tatctcagttcggtgtaggtcgttcgctccaagct |
| | gggctgtgtgcacgaaccccccgttcagcccgacc |
| | gctgcgccttatccggtaactatcgtcttgagtcc |
| | aacccggtaagacacgacttatcgccactggcagc |
| | agccactggtaacaggattagcagagcgaggtatg |
| | taggcggtgctacagagttcttgaagtggtggcct |
| | aactacggctacactagaagaacagtatttggtat |
| | ctgcgctctgctgaagccagttaccttcggaaaaa |
| | gagttggtagctcttgatccggcaaacaaaccacc |
| | gctggtagcggtggtttttttgtttgcaagcagca |
| | gattacgcgcagaaaaaaaggatctcaagaagatc |
| | ctttgatcttttctacgggtaccgttcgtataatg |
| | tatgctatacgaagttatTCAGAGGTTTTCACCGT |
| | CATCACCGAAACGCGCGAGGCAGCTGCGGTAAAGC |
| | TCATCAGCGTGGTCGTGAAGCGATTCACAGATGTC |
| | TGCCTGTTCATCCGCGTCCAGCTCGTTGAGTTTCT |
| | CCAGAAGCGTTAATGTCTGGCTTCTGATAAAGCGG |
| | GCCATGTTAAGGGCGGTTTTTTCCTGTTTGGTCAC |
| | gtctgacgctcagtggaacgaaaactcacgttaag |
| | ggattttggtcatgagattatcaaaaaggatcttc |
| | acctagatccttttaaattaaaaatgaagttttaa |
| | atcaagcccaatctgaataatgttacaaccaatta |
| | accaattctgattagaaaaactcatcgagcatcaa |
| | atgaaactgcaatttattcatatcaggattatcaa |
| | taccatatttttgaaaaagccgtttctgtaatgaa |
| | ggagaaaactcaccgaggcagttccataggatggc |
| | aagatcctggtatcggtctgcgattccgactcgtc |
| | caacatcaatacaacctattaatttcccctcgtca |
| | aaaataaggttatcaagtgagaaatcaccatgagt |
| | gacgactgaatccggtgagaatggcaaaagtttat |
| | gcatttctttccagacttgttcaacaggccagcca |
| | ttacgctcgtcatcaaaatcactcgcatcaaccaa |
| | accgttattcattcgtgattgcgcctgagcgagac |
| | gaaatacgcgatcgctgttaaaaggacaattacaa |
| | acaggaatcgaatgcaaccggcgcaggaacactgc |
| | cagcgcatcaacaatattttcacctgaatcaggat |
| | attcttctaatacctggaatgctgtttttccgggg |
| | atcgcagtggtgagtaaccatgcatcatcaggagt |
| | acggataaaatgcttgatggtcggaagaggcataa |
| | attccgtcagccagtttagtctgaccatctcatct |
| | gtaacatcattggcaacgctacctttgccatgttt |
| | cagaaacaactctggcgcatcgggcttcccataca |
| | agcgatagattgtcgcacctgattgcccgacatta |
| | tcgcgagcccatttatacccatataaatcagcatc |
| | catgttggaatttaatcgcggcctcgacgtttccc |
| | gttgaatatggctcataacaccccttgtattactg |
| | tttatgtaagcagacagttttattgttcatgatga |
| | tatatttttatcttgtgcaatgtaacatcagagat |
| | tttgagacacgggccagagctgca |
| | (SEQ ID NO: 41) |
| pMF5-loxp | TTCTCATGTTTGACAGCTTATCATCGATAAGCTTT |
| precursor | AATGCGGTAGTTTATCACAGTTAAATTGCTAACGC |
| empty | AGTCAGGCACCGTGTATGGAAGCCGGCGGCACCTC |
| vector | GCTAACGGATTCACCACTCCAAGAATTGGAGCCAA |
| plasmid | TCAATTCTTGCGGAGAACTGTGAATGCGCAAACCA |
| | ACCCTTGGCAGAACATATCCATCGCGTCCGCCATC |
| | TCCAGCAGCCGCACGCGGCGCATCTCGGGCAGCGT |
| | TGGGTCCTGGCCACGGGTGCGCATGATCGTGCTCC |
| | TGTCGTTGAGGACCCGGCTAGGCTGGCGGGGTTGC |
| | CTTACTGGTTAGCAGAATGAATCACCGATACGCGA |
| | GCGAACGTGAAGCGACTGCTGCTGCAAAACGTCTG |
| | CGACCTGAGCAACAACATGAATGGTCTTCGGTTTC |
| | CGTGTTTCGTAAAGTCTGGAAACGCGGAAGTCAGC |
| | GCCCTGCACCATTATGTTCCGGATCTGCATCGCAG |
| | GATGCTGCTGGCTACCCTGTGGAACACCTACATCT |
| | GTATTAACGAAGCGCTGGCATTGACCCTGAGTGAT |
| | TTTTCTCTGGTCCCGCCGCATCCATACCGCCAGTT |
| | GTTTACCCTCACAACGTTCCAGTAACCGGGCATGT |
| | TCATCATCAGTAACCCGTATCGTGAGCATCCTCTC |
| | TCGTTTCATCGGTATCATTACCCCCATGAACAGAA |
| | ATCCCCCTTACACGGAGGCATCAataacttcgtat |
| | aatgtatgctatacgaacggtaCTGCA Gtgtcag |
| | ccgttaagtgttcctgtgtcactcaaaattgcttt |
| | gagaggctctaagggcttctcagtgcgttacatcc |
| | ctggcttgttgtccacaaccgttaaaccttaaaag |
| | ctttaaaagccttatatattcttttttttcttata |
| | aaacttaaaaccttagaggctatttaagttgctga |
| | tttatattaattttattgttcaaacatgagagctt |
| | agtacgtgaaacatgagagcttagtacgttagcca |
| | tgagagcttagtacgttagccatgagggtttagtt |
| | cgttaaacatgagagcttagtacgttaaacatgag |
| | agcttagtacgtgaaacatgagagcttagtacgta |
| | ctatcaacaggttgaactgctgatcttcagatcGG |
| | ATCCTCTAGAGTCGACGCATGCtaccgttcgtata |
| | atgtatgctatacgaagttatTGATCTTTTCTACG |
| | GGGTCTGACGCTCAGTGGAACGAAAACTCACGTTA |
| | AGGGATTTTGGTCATGAGATTATCAAAAAGGATCT |
| | TCACCTAGATCCTTTTAAATTAAAAATGAAGTTTT |
| | AAATCAATCTAAAGTATATATGAGTAAACTTGGTC |
| | TGACAGttagaaaaactcatcgagcatcaaatgaa |
| | actgcaatttattcatatcaggattatcaatacca |
| | tatttttgaaaaagccgtttctgtaatgaaggaga |
| | aaactcaccgaggcagttccataggatggcaagat |
| | cctggtatcggtctgcgattccgactcgtccaaca |
| | tcaatacaacctattaatttcccctcgtcaaaaat |
| | aaggttatcaagtgagaaatcaccatgagtgacga |
| | ctgaatccggtgagaatggcaaaagtttatgcatt |
| | tctttccagacttgttcaacaggccagccattacg |
| | ctcgtcatcaaaatcactcgcatcaaccaaaccgt |
| | tattcattcgtgattgcgcctgagcgagacgaaat |
| | acgcgatcgctgttaaaaggacaattacaaacagg |
| | aatcgaatgcaaccggcgcaggaacactgccagcg |
| | catcaacaatattttcacctgaatcaggatattct |
| | tctaatacctggaatgctgttttcccggggatcgc |
| | agtggtgagtaaccatgcatcatcaggagtacgga |
| | taaaatgcttgatggtcggaagaggcataaattcc |
| | gtcagccagtttagtctgaccatctcatctgtaac |
| | atcattggcaacgctacctttgccatgtttcagaa |
| | acaactctggcgcatcgggcttcccatacaatcga |
| | tagattgtcgcacctgattgcccgacattatcgcg |
| | agcccatttatacccatataaatcagcatccatgt |
| | tggaatttaatcgcggcctagagcaagacgtttcc |
| | cgttgaatatggctcatCACATTTCCCCGAAAAGT |
| | GCCACCTGACGTCTAAGAAACCATTATTATCATGA |
| | CATTAACCTATAAAAATAGGCGTATCACGAGGCCC |
| | TTTCGTCTTCAAGAA |
| | (SEQ ID NO: 42) |
| pMB1 ori | CGCTCTTCCGCTTCCTCGCTCACTGACTCGCTGCG |
| derivative | CTCGGTCGTTCGGCTGCGGCGAGCGGTATCAGCTC |
| (784 bp) | ACTCAAAGGCGGTAATACGGTTATCCACAGAATCA |
| | GGGGATAACGCAGGAAAGAACATGTGAGCAAAAGG |
| | CCAGCAAAAGGCCAGGAACCGTAAAAAGGCCGCGT |
| | TGCTGGCGTTTTTCCATAGGCTCCGCCCCCCTGAC |
| | GAGCATCACAAAAATCGACGCTCAAGTCAGAGGTG |
| | GCGAAACCCGACAGGACTATAAAGATACCAGGCGT |
| | TTCCCCCTGGAAGCTCCCTCGTGCGCTCTCCTGTT |
| | CCGACCCTGCCGCTTACCGGATACCTGTCCGCCTT |
| | TCTCCCTTCGGGAAGCGTGGCGCTTTCTCATAGCT |
| | CACGCTGTAGGTATCTCAGTTCGGTGTAGGTCGTT |
| | CGCTCCAAGCTGGGCTGTGTGCACGAACCCCCCGT |
| | TCAGCCCGACCGCTGCGCCTTATCCGGTAACTATC |
| | GTCTTGAGTCCAACCCGGTAAGACACGACTTATCG |
| | CCACTGGCAGCAGCCACTGGTAACAGGATTAGCAG |
| | AGCGAGGTATGTAGGCGGTGCTACAGAGTTCTTGA |
| | AGTGGTGGCCTAACTACGGCTACACTAGAAGGACA |
| | GTATTTGGTATCTGCGCTCTGCTGAAGCCAGTTAC |
| | CTTCGGAAAAAGAGTTGGTAGCTCTTGATCCGGCA |
| | AACAAACCACCGCTGGTAGCGGTGGTTTTTTTGTT |
| | TGCAAGCAGCAGATTACGCGCAGAAAAAAAGGATC |
| | TCAAGAAGATCCTT |
| | (SEQ ID NO: 43) |
| pMB1 ori | tttccataggctccgcccccctgacgagcatcaca |
| (589 bp) | aaaatcgacgctcaagtcagaggtggcgaaacccg |
| | acaggactataaagataccaggcgtttccccctgg |
| | aagctccctcgtgcgctctcctgttccgaccctgc |
| | cgcttaccggatacctgtccgcctttctcccttcg |
| | ggaagcgtggcgctttctcatagctcacgctgtag |
| | gtatctcagttcggtgtaggtcgttcgctccaagc |
| | tgggctgtgtgcacgaaccccccgttcagcccgac |
| | cgctgcgccttatccggtaactatcgtcttgagtc |
| | caacccggtaagacacgacttatcgccactggcag |
| | cagccactggtaacaggattagcagagcgaggtat |
| | gtaggcggtgctacagagttcttgaagtggtggcc |
| | taactacggctacactagaagaacagtatttggta |
| | tctgcgctctgctgaagccagttaccttcggaaaa |
| | agagttggtagctcttgatccggcaaacaaaccac |
| | cgctggtagcggtggtttttttgtttgcaagcagc |
| | agattacgcgcagaaaaaaaggatctcaa |
| | (SEQ ID NO: 44) |
| pUC ori | atgtgagcaaaaggccagcaaaaggccaggaaccg |
| (674 bp) | taaaaaggccgcgttgctggcgtttttccataggc |
| | tccgcccccctgacgagcatcacaaaaatcgacgc |
| | tcaagtcagaggtggcgaaacccgacaggactata |
| | aagataccaggcgtttccccctggaagctccctcg |
| | tgcgctctcctgttccgaccctgccgcttaccgga |
| | tacctgtccgcctttctcccttcgggaagcgtggc |
| | gctttctcatagctcacgctgtaggtatctcagtt |
| | cggtgt |
| | aggtcgttcgctccaagctgggctgtgtgcagaac |
| | cccccgttcagcccgaccgctgcgccttatccggt |
| | aactatcgtcttgagtccaacccggtaagacacga |
| | cttatcgccactggcagcagccactggtaacagga |
| | ttagcagagcgaggtatgtaggcggtgctacagag |
| | ttcttgaagtggtggcctaactacggctacactag |
| | aagaacagtatttggtatctgcgctctgctgaagc |
| | cagttaccttcggaaaaagagttggtagctcttga |
| | tccggcaaacaaaccaccgctggtagcggtggttt |
| | ttttgtttgcaagcagcagattacgcgcagaaaaa |
| | aaggatctcaagaagatcctitgatcttttctacg |
| | gg(SEQ ID NO: 45) |
| R6Kγ ori | Tgtcagccgttaagtgttcctgtgtcactcaaaat |
| (389 bp) | tgctttgagaggctctaagggcttctcagtgcgtt |
| | acatccctggcttgttgtccacaaccgttaaacct |
| | taaaagctttaaaagccttatatattctttttttt |
| | cttataaaacttaaaaccttagaggctatttaagt |
| | tgctgatttatattaattttattgttcaaacatga |
| | gagcttagtacgtgaaacatgagagcttagtacgt |
| | tagccatgagagcttagtacgttagccatgagggt |
| | ttagttcgttaaacatgagagcttagtacgttaaa |
| | catgagagcttagtacgtgaaacatgagagcttag |
| | tacgtactatcaacaggttgaactgctgatcttca |
| | gatc |
| | (SEQ ID NO: 46) |
| pMF9-FRT | TTCTCATGTTTGACAGCTTATCATCGATAAGCTTT |
| precursor | AATGCGGTAGTTTATCACAGTTAAATTGCTAACGC |
| empty | AGTCAGGCACCGTGTATGGAAGCCGGCGGCACCTC |
| vector | GCTAACGGATTCACCACTCCAAGAATTGGAGCCAA |
| plasmid | TCAATTCTTGCGGAGAACTGTGAATGCGCAAACCA |
| | ACCCTTGGCAGAACATATCCATCGCGTCCGCCATC |
| | TCCAGCAGCCGCACGCGGCGCATCTCGGGCAGCGT |
| | TGGGTCCTGGCCACGGGTGCGCATGATCGTGCTCC |
| | TGTCGTTGAGGACCCGGCTAGGCTGGCGGGGTTGC |
| | CTTACTGGTTAGCAGAATGAATCACCGATACGCGA |
| | GCGAACGTGAAGCGACTGCTGCTGCAAAACGTCTG |
| | CGACCTGAGCAACAACATGAATGGTCTTCGGTTTC |
| | CGTGTTTCGTAAAGTCTGGAAACGCGGAAGTCAGC |
| | GCCCTGCACCATTATGTTCCGGATCTGCATCGCAG |
| | GATGCTGCTGGCTACCCTGTGGAACACCTACATCT |
| | GTATTAACGAAGCGCTGGCATTGACCCTGAGTGAT |
| | TTTTCTCTGGTCCCGCCGCATCCATACCGCCAGTT |
| | GTTTACCCTCACAACGTTCCAGTAACCGGGCATGT |
| | TCATCATCAGTAACCCGTATCGTGAGCATCCTCTC |
| | TCGTTTCATCGGTATCATTACCCCCATGAACAGAA |
| | ATCCCCCTTACACGGAGGCATCAGTGACCAAACAG |
| | GAAAAAACCGCCCTTAACATGGCCCGCTTTATCAG |
| | AAGCCAGACATTAACGCTTCTGGAGAAACTCAACG |
| | AGCTGGACGCGGATGAACAGGCAGACATCTGTGAA |
| | TCGCTTCACGACCACGCTGATGAGCTTTACCGCAG |
| | CTGCCTCGCGCGTTTCGGTGATGACGGTGAAAACC |
| | TCTGACACATGCAGCTCCCGGAGACGGTCACAGCT |
| | TGTCTGTAAGCGGATGCCGGGAGCAGACAAGCCCG |
| | TCAGGGCGCGTCAGCGGGTGTTGGCGGGTGTCGGG |
| | GCGCAGCCATGACCCAGTCACGTAGCGATAGCGGA |
| | GTGTATACTGGCTTAACTATGCGGCATCAGAGCAG |
| | ATTGTACTGAGAGTGCACCATATGCGGTGTGAAAT |
| | ACCGCACAGATGCGTAAGGAGAAAATACCGCATCA |
| | GGgaagttcctatactttctagagaataggaactt |
| | cggaataggaacttcCGCTCTTCCGCTTCCTCGCT |
| | CACTGACTCGCTGCGCTCGGTCGTTCGGCTGCGGC |
| | GAGCGGTATCAGCTCACTCAAAGGCGGTAATACGG |
| | TTATCCACAGAATCAGGGGATAACGCAGGAAAGAA |
| | CATGTGAGCAAAAGGCCAGCAAAAGGCCAGGAACC |
| | GTAAAAAGGCCGCGTTGCTGGCGTTTTTCCATAGG |
| | CTCCGCCCCCCTGACGAGCATCACAAAAATCGACG |
| | CTCAAGTCAGAGGTGGCGAAACCCGACAGGACTAT |
| | AAAGATACCAGGCGTTTCCCCCTGGAAGCTCCCTC |
| | GTGCGCTCTCCTGTTCCGACCCTGCCGCTTACCGG |
| | ATACCTGTCCGCCTTTCTCCCTTCGGGAAGCGTGG |
| | CGCTTTCTCATAGCTCACGCTGTAGGTATCTCAGT |
| | TCGGTGTAGGTCGTTCGCTCCAAGCTGGGCTGTGT |
| | GCACGAACCCCCCGTTCAGCCCGACCGCTGCGCCT |
| | TATCCGGTAACTATCGTCTTGAGTCCAACCCGGTA |
| | AGACACGACTTATCGCCACTGGCAGCAGCCACTGG |
| | TAACAGGATTAGCAGAGCGAGGTATGTAGGCGGTG |
| | CTACAGAGTTCTTGAAGTGGTGGCCTAACTACGGC |
| | TACACTAGAAGGACAGTATTTGGTATCTGCGCTCT |
| | GCTGAAGCCAGTTACCTTCGGAAAAAGAGTTGGTA |
| | GCTCTTGATCCGGCAAACAAACCACCGCTGGTAGC |
| | GGTGGTTTTTTTGTTTGCAAGCAGCAGATTACGCG |
| | CAGAAAAAAAGGATCTCAAGAAGATCCTTGTAAAA |
| | CGACGGCCAGTGAATTCGAGCTCGGTACCCGGGGA |
| | TCCTCTAGAGTCGACCTGCAGGCATGCAAGCTTGG |
| | CGTAATCATGGTCATAGCTGTTTCCTGgaagttcc |
| | tatactttctagagaataggaacttcggaataggt |
| | acttcTGATCTTTTCTACGGGGTCTGACGCTCAGT |
| | GGAACGAAAACTCACGTTAAGGGATTTTGGTCATG |
| | AGATTATCAAAAAGGATCTTCACCTAGATCCTTTT |
| | AAATTAAAAATGAAGTTTTAAATCAATCTAAAGTA |
| | TATATGAGTAAACTTGGTCTGACAGttagaaaaac |
| | tcatcgagcatcaaatgaaactgcaatttattcat |
| | atcaggattatcaataccatatttttgaaaaagcc |
| | gtttctgtaatgaaggagaaaactcaccgaggcag |
| | ttccataggatggcaagatcctggtatcggtctgc |
| | gattccgactcgtccaacatcaatacaacctatta |
| | atttcccctcgtcaaaaataaggttatcaagtgag |
| | aaatcaccatgagtgacgactgaatccggtgagaa |
| | tggcaaaagtttatgcatttctttccagacttgtt |
| | caacaggccagccattacgctcgtcatcaaaatca |
| | ctcgcatcaaccaaaccgttattcattcgtgattg |
| | cgcctgagcgagacgaaatacgcgatcgctgttaa |
| | aaggacaattacaaacaggaatcgaatgcaaccgg |
| | gcaggaacactgccagcgcatcaacaatattttca |
| | cctgaatcaggatattcttctaatacctggaatgc |
| | tgttttcccggggatcgcagtggtgagtaaccatg |
| | catcatcaggagtacggataaaatgcttgatggtc |
| | ggaagaggcataaattccgtcagccagtttagtct |
| | gaccatctcatctgtaacatcattggcaacgctac |
| | ctttgccatgtttcagaaacaactctggcgcatcg |
| | ggcttcccatacaatcgatagattgtcgcacctga |
| | ttgcccgacattatcgcgagcccatttatacccat |
| | ataaatcagcatccatgttggaatttaatcgcggc |
| | ctagagcaagacgtttcccgttgaatatggctcat |
| | CACATTTCCCCGAAAAGTGCCACCTGACGTCTAAG |
| | AAACCATTATTATCATGACATTAACCTATAAAAAT |
| | AGGCGTATCACGAGGCCCTTTCGTCTTCAAGAA |
| | (SEQ ID NO: 47) |
| attB | GGTGCCAGGGCGTGCCCTTGGGCTCCCCGGGCGCG |
| sequence | (SEQ ID NO:48) |
| attP | CCCAACTGGGGTAACCTTTGAGTTCTCTCAGTTGG |
| sequence | GG |
| | (SEQ ID NO: 49) |
| phiC31: | GTGGACACGTACGCGGGTGCTTACGACCGTCAGTC |
| (protein | GCGCGAGCGCGAAAATTCGAGCGCAGCAAGCCCAG |
| ID: | CGACACAGCGTAGCGCCAACGAAGACAAGGCGGCC |
| ADD10761.1) | GACCTTCAGCGCGAAGTCGAGCGCGACGGGGGCCG |
| | GTTCAGGTTCGTCGGGCATTTCAGCGAAGCGCCGG |
| | GCACGTCGGCGTTCGGGACGGCGGAGCGCCCGGAG |
| | TTCGAACGCATCCTGAACGAATGCCGCGCCGGGCG |
| | GCTCAACATGATCATTGTCTATGACGTGTCGCGCT |
| | TCTCGCGCCTGAAGGTCATGGACGCGATTCCGATT |
| | GTCTCGGAATTGCTCGCCCTGGGCGTGACGATTGT |
| | TTCCACTCAGGAAGGCGTCTTCCGGCAGGGAAACG |
| | TCATGGACCTGATTCACCTGATTATGCGGCTCGAC |
| | GCGTCGCACAAAGAATCTTCGCTGAAGTCGGCGAA |
| | GATTCTCGACACGAAGAACCTTCAGCGCGAATTGG |
| | GCGGGTACGTCGGCGGGAAGGCGCCTTACGGCTTC |
| | GAGCTTGTTTCGGAGACGAAGGAGATCACGCGCAA |
| | CGGCCGAATGGTCAATGTCGTCATCAACAAGCTTG |
| | CGCACTCGACCACTCCCCTTACCGGACCCTTCGAG |
| | TTCGAGCCCGACGTAATCCGGTGGTGGTGGCGTGA |
| | GATCAAGACGCACAAACACCTTCCCTTCAAGCCGG |
| | GCAGTCAAGCCGCCATTCACCCGGGCAGCATCACG |
| | GGGCTTTGTAAGCGCATGGACGCTGACGCCGTGCC |
| | GACCCGGGGCGAGACGATTGGGAAGAAGACCGCTT |
| | CAAGCGCCTGGGACCCGGCAACCGTTATGCGAATC |
| | CTTCGGGACCCGCGTATTGCGGGCTTCGCCGCTGA |
| | GGTGATCTACAAGAAGAAGCCGGACGGCACGCCGA |
| | CCACGAAGATTGAGGGTTACCGCATTCAGCGCGAC |
| | CCGATCACGCTCCGGCCGGTCGAGCTTGATTGCGG |
| | ACCGATCATCGAGCCCGCTGAGTGGTATGAGCTTC |
| | AGGCGTGGTTGGACGGCAGGGGGCGCGGCAAGGGG |
| | CTTTCCCGGGGGCAAGCCATTCTGTCCGCCATGGA |
| | CAAGCTGTACTGCGAGTGTGGCGCCGTCATGACTT |
| | CGAAGCGCGGGGAAGAATCGATCAAGGACTCTTAC |
| | CGCTGCCGTCGCCGGAAGGTGGTCGACCCGTCCGC |
| | ACCTGGGCAGCACGAAGGCACGTGCAACGTCAGCA |
| | TGGCGGCACTCGACAAGTTCGTTGCGGAACGCATC |
| | TTCAACAAGATCAGGCACGCCGAAGGCGACGAAGA |
| | GACGTTGGCGCTTCTGTGGGAAGCCGCCCGACGCT |
| | TCGGCAAGCTCACTGAGGCGCCTGAGAAGAGCGGC |
| | GAACGGGCGAACCTTGTTGCGGAGCGCGCCGACGC |
| | CCTGAACGCCCTTGAAGAGCTGTACGAAGACCGCG |
| | CGGCAGGCGCGTACGACGGACCCGTTGGCAGGAAG |
| | CACTTCCGGAAGCAACAGGCAGCGCTGACGCTCCG |
| | GCAGCAAGGGGCGGAAGAGCGGCTTGCCGAACTTG |
| | AAGCCGCCGAAGCCCCGAAGCTTCCCCTTGACCAA |
| | TGGTTCCCCGAAGACGCCGACGCTGACCCGACCGG |
| | CCCTAAGTCGTGGTGGGGGCGCGCGTCAGTAGACG |
| | ACAAGCGCGTGTTCGTCGGGCTCTTCGTAGACAAG |
| | ATCGTTGTCACGAAGTCGACTACGGGCAGGGGGCA |
| | GGGAACGCCCATCGAGAAGCGCGCTTCGATCACGT |
| | GGGCGAAGCCGCCGACCGACGACGACGAAGACGAC |
| | GCCCAGGACGGCACGGAAGACGTAGCGGCGTAG |
| | (SEQ ID NO: 50) |

Some primer sequence information mentioned in this specification is as follows:

| Primer | | |
|---|---|---|
| name | Feature | Sequence and sequence No. |
| cre-F | Amplification | CTGTTTCTCCATACCCGTTTTTTTGGGATGcaccatcac |
| | | catcaccatATGTCCAATTTACTGA (SEQ ID NO: 2) |
| cre-R | primer of | GGGGATTCAGTAACATTCACGCCGGAAGTGGAAT |
| | cre gene | TCTTAATCGCCATCTTCCAGCA (SEQ ID NO: 3) |
| cre-seq1 | Sequencing primer | ATTATTTGCACGGCGTCACA (SEQ ID NO: 4) |
| | of pSC101- | |
| | araBAD-cre(Amp) | |
| | plasmid | |
| cre-seq2 | | AACGGGCATTTCAGTTCAAG (SEQ ID NO: 5) |
| pSC101 | Amplification | GCCAATACCAGTAGAAACAGACGAAGAATCGGTA |
| (ts)-F | primer of pSC101 | TGGACAGTTTTCCCTT (SEQ ID NO: 6) |
| | ori (ts) fragment | |
| pSC101 | | AAAGGAATATTCAGCGATTTGCCCGATTGCGCAAC |
| (ts)-R | | CGAGCTTGCGAGGGT (SEQ ID NO: 7) |
| 5.5kb-F | Amplification | AAGCAGCAGATTACGCGCAGAAAAAAAGGATCTC |
| | primer of 5.5 kb | AAGAAGATCCTTGTAAAACGACGGCCAGTGAATT |
| | fragment | CGgacattgattattgactagt (SEQ ID NO: 12) |
| 5.5kb-R | | acgaacggtaCAGGAAACAGCTATGACCATGATTACGC |
| | | CAAGCTTGCATGCCTGCAGGTCGACTCTAGAGccat |
| | | agagcccaccgcatcc (SEQ ID NO: 13) |
| C4818FK0 | Bacteria detection | AGCGTGGAAGTCTTCTGTTATAGCCA |
| 60-6JJF1 | primer of strain | (SEQ ID NO: 14) |
| | JM108 araBAD- | |
| C4818FK0 | cre after gene | ACCTCGCGCCAGGATGGCTTTATGCAC |
| 60-6JJR1 | editing | (SEQ ID NO: 15) |
| flp-F | Amplification | TTTTTTTGGGATGcaccatcaccatcaccatatgccacaatttga |
| | | tatat (SEQ ID NO: 16) |
| flp-R | primer of flp gene | TTCAGTAACATTCACGCCGGAAGTGGAATTttatatgc |
| | | gtctatttatgt (SEQ ID NO: 17) |
| FRT-F1 | Amplification | CGTAAGGAGAAAATACCGCATCAGGgaagttcctatactttcta |
| | primer of FRT-RFP | gagaataggaacttcggaataggaacttcCGCTCTTCCGCTTCC |
| | fragment | TCGCT (SEQ ID NO: 18) |
| FRT-R1 | | GCGTCAGACCCCGTAGAAAAGATCAgaagtacctattccgaagt |
| | | tcctattctctagaaagtataggaacttcCAGGAAACAGCTATG |
| | | ACCAT (SEQ ID NO: 19) |
| FRT-F2 | Amplification | TGATCTTTTCTACGGGGTCT (SEQ ID NO: 20) |
| | primer of FRT | |
| | vector | |
| FRT-R2 | | CCTGATGCGGTATTTTCTCC (SEQ ID NO: 21) |
| pUC57- | pMB1 | ataacttcgtatagcatacattatacgaacggtattgagatccttt |
| short-1-1 | amplification | ttttctgcgcgtaatctg (SEQ ID NO: 30) |
| | primer | |
| pUC57- | | gctatacgaacggtagaattcggatcccgggcccgtcgactgcagt |
| short-1-2 | | ttccataggctccgccccc (SEQ ID NO: 31) |
| pUC57- | Amplification | ccgggatccgaattctaccgttcgtatagcatacattatacgaagt |
| short-2-1 | primer of | tattctggccgtcgttttacaacg (SEQ ID NO: 32) |
| | pUC57(Kan^{R}) | |
| | fragment | |
| pUC57- | | gttcgtataatgtatgctatacgaagttatgaagatcctttgatct |
| short-2-2 | | tttc (SEQ ID NO: 33) |
| Short-F | Amplification | gggatccgaattctaccgttcgtatagcatacattatacgaagtta |
| | primer of pMF65- | ttct (SEQ ID NO: 35) |
| | loxp empty vector | |
| Short-R | | gggcccgtcgactgcagtttccataggctccgcccccctgacgagc |
| | | atcacaaa (SEQ ID NO: 36) |
| RFP-F | Amplification | atgctatacgaacggtagaattcggatcccGTACCCAATACGCAAA |
| | primer of RFP | CCGC (SEQ ID NO: 37) |
| | fragment | |
| RFP-R | | gagcctatggaaactgcagtcgacgggcccGGCCGCTCGAGTCG |
| | | ACTATA (SEQ ID NO: 38) |

### REFERENCES

[1] Davies, J; Smith, D I (1978). Plasmid-Determined Resistance to Antimicrobial Agents. Annual Review of Microbiology, 32(1), 469-508
[2] Schleef, M. (2013) Minicircle and Miniplasmid DNA Vectors: The Future of Non-Viral and Viral Gene Transfer. Wiley-VCH
[3] Luke, J. et al. (2009) Improved antibiotic-free DNA vaccine vectors utilizing a novel RNA based plasmid selection system. Vaccine 27, 6454-6459

1. A precursor plasmid, comprising:
1) a replication original site;
2) a selectable marker gene;
3) a target gene or a cloning site for inserting the target gene; and
4) paired recombination sites, wherein
the paired recombination sites enable the precursor plasmid to perform self-recombination in the presence of recombinase to form a molecule of daughter plasmid without the selectable marker gene and a molecule of circular double-stranded DNA;
the daughter plasmid comprises the replication original site and the target gene, or comprises the replication original site and the cloning site; and
the circular double-stranded DNA comprises the selectable marker gene.

2. The precursor plasmid according to claim 1, wherein the sequences of the paired recombination sites are in the same direction.

3. The precursor plasmid according to claim 1, wherein the replication original site is adjacent to the target gene or the cloning site, the paired recombination sites are respectively adjacent to upstream and downstream of the replication original site and the target gene, or the paired recombination sites are respectively adjacent to upstream and downstream of the replication original site and the cloning site.

4. The precursor plasmid according to claim 1, wherein the paired recombination sites are selected from the loxP sequence in the same direction, the FRT sequence in the same direction, and the attB/attP sequence in the same direction.

5. The precursor plasmid according to claim 4, wherein the paired recombination sites are the lox71 sequence and the lox66 sequence in the same direction.

6. The precursor plasmid according to claim 1, wherein the replication original site is selected from a replication original site for the pUC, a replication original site for the pMB1 and derivatives thereof, a replication original site for the ColE1, and a replication original site for the R6Kγ.

7. The precursor plasmid according to claim 6, wherein the replication original site comprises the following sequences: nucleotide sequences as set forth in SEQ ID NOs: 43-46 and nucleotide sequences that have at least 80% identity with the nucleotide sequences as set forth in SEQ ID NOs: 43-46 and can function as the replication origin.

8. The precursor plasmid according to claim 1, further comprising one or more of the following sequences: a rop gene sequence, a coding sequence of endonuclease, and a coding sequence of plasmid replication accessory protein.

9. The precursor plasmid according to claim 1, wherein the selectable marker gene is an antibiotic resistant gene.

10. (canceled)

11. The precursor plasmid according to claim 1, further comprising the coding gene of the recombinase.

12. The precursor plasmid according to claim 11, wherein the precursor plasmid can express the recombinase under suitable conditions.

13. (canceled)

14. The precursor plasmid according to claim 1, comprising a nucleotide sequence as set forth in SEQ ID NO: 8, 34, 39-42, or 47 or a nucleotide sequence that has at least 80% identity with the nucleotide sequence as set forth in SEQ ID NO: 8, 34, 39-42, or 47.

15. (canceled)

16. A method for preparing a daughter plasmid without selectable marker gene, comprising:
1) introducing the precursor plasmid according to claim 1 into a host cell that can express or can help express the recombinase, and screening out host cells that express the selectable marker gene;
2) culturing the host cells screened out in step 1) to allow the recombinase to be expressed in the host cells, and culturing the host cells and screening out host cells that do not express the selectable marker gene; and
3) culturing the host cells screened out in step 2) and extracting plasmids to obtain the daughter plasmid.

17. The method according to claim 16, wherein the paired recombination sites are the loxP sequence in the same direction, and the recombinase is the Cre recombinase; the paired recombination sites are the FRT sequence in the same direction, and the recombinase is the Flp recombinase; or the paired recombination sites are the attB/attP sequence in the same direction, and the recombinase is the PhiC31 recombinase.

18. The method according to claim 16, wherein the paired recombination sites are the lox71 sequence and the lox66 sequence in the same direction, and the recombinase is the Cre recombinase.

19. The method according to claim 16, wherein the host cell comprises the coding gene of the recombinase in the genome thereof, or the host cell comprises an expression vector containing the coding gene of the recombinase.

20. The method according to claim 16, wherein the precursor plasmid comprises the coding gene of the recombinase, and the precursor plasmid can express the recombinase in the host cell.

21. The method according to claim 16, wherein the recombinase is inducibly expressed in the host cell.

22. The method according to claim 19, wherein the expression vector comprises a conditionally induced loss-type plasmid replication element.

23. The method according to claim 16, wherein the host cell is E. coli.

24. A kit for preparing a daughter plasmid without selectable marker gene, comprising the precursor plasmid according to claim 1, and a host cell that can express or can help express the recombinase.

25. (canceled)

26. The kit according to claim 24, wherein the paired recombination sites are the loxP sequence in the same direction, and the recombinase is the Cre recombinase; the paired recombination sites are the FRT sequence in the same direction, and the recombinase is the Flp recombinase; or the paired recombination sites are the attB/attP sequence in the same direction, and the recombinase is the phiC31 recombinase.

27. The kit according to claim 24, wherein the paired recombination sites are the lox71 sequence and the lox66 sequence in the same direction, and the recombinase is the Cre recombinase.

28. The kit according to claim 24, wherein the host cell comprises the coding gene of the recombinase in the genome thereof, or the host cell comprises an expression vector containing the coding gene of the recombinase.

29. (canceled)

30. The kit according to claim 28, wherein the expression vector comprises a conditionally induced loss-type plasmid replication element.

31. The kit according to claim 24, wherein the host cell is E. coli.

32. (canceled)

33. A daughter plasmid, comprising a replication original site and a target gene, wherein the daughter plasmid does not comprise an antibiotic resistant gene.

34. The daughter plasmid according to claim 33, wherein the replication original site is selected from a replication original site for the pUC, a replication original site for the pMB1 and derivatives thereof, a replication original site for the ColE1, and a replication original site for the R6Kγ.

35. The daughter plasmid according to claim 33, wherein the replication original site comprises the following sequences: nucleotide sequences as set forth in SEQ ID NOs: 43-46 and nucleotide sequences that have at least 80% identity with the nucleotide sequences as set forth in SEQ ID NOs: 43-46 and can function as the replication origin.

36. The daughter plasmid according to claim 33, wherein the target gene comprises one or more of the following sequences: a promoter, a coding gene of an expressed protein, and a terminator.

37. The daughter plasmid according to claim 33, being capable of replicating in a host cell.

38. The daughter plasmid according to claim 37, being capable of replicating in the host cell under a cell culture condition without screening stress.

39. A daughter plasmid obtained by the method according to claim 16.

40. A host cell comprising the daughter plasmid according to claim 33, wherein the daughter plasmid can replicate in the host cell.

41. (canceled)

42. The host cell according to claim 40, enabling amplification of the daughter plasmid under a cell culture condition without screening stress.

43. A composition, comprising the daughter plasmid according to claim 33, wherein the content of the daughter plasmid is at least 80%, 85%, 90%, 95%, 96%, 97%, 98%, or 99%, wherein a method for determining a content of the daughter plasmid in the composition is an NGS analysis method or a gel electrophoresis imaging analysis method.

44.-46. (canceled)


SEQUENCE LISTING (50 sequences)

SEQ ID NO: 1 - DNA, 1050 bp, Artificial Sequence
  cre gene
caccatcaccatcaccatatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaa

SEQ ID NO: 2 - DNA, 64 bp, Artificial Sequence
  Amplification primer of cre gene cre-F
ctgtttctccatacccgtttttttgggatgcaccatcaccatcaccatatgtccaatttactga

SEQ ID NO: 3 - DNA, 56 bp, Artificial Sequence
  Amplification primer of cre gene cre-R
ggggattcagtaacattcacgccggaagtggaattcttaatcgccatcttccagca

SEQ ID NO: 4 - DNA, 20 bp, Artificial Sequence
  Sequencing primer of pSC101-araBAD-cre(Amp) plasmid cre-seq1
attatttgcacggcgtcaca

SEQ ID NO: 5 - DNA, 20 bp, Artificial Sequence
  Sequencing primer of pSC101-araBAD-cre(Amp) plasmid cre-seq2
aacgggcatttcagttcaag

SEQ ID NO: 6 - DNA, 50 bp, Artificial Sequence
  Amplification primer of pSC101 ori (ts) fragment pSC101(ts)-F
gccaataccagtagaaacagacgaagaatcggtatggacagttttccctt

SEQ ID NO: 7 - DNA, 50 bp, Artificial Sequence
  Amplification primer of pSC101 ori (ts) fragment pSC101(ts)-R
aaaggaatattcagcgatttgcccgattgcgcaaccgagcttgcgagggt

SEQ ID NO: 8 - DNA, 3191 bp, Artificial Sequence
  pMF9-loxp precursor empty vector plasmid
ttctcatgtttgacagcttatcatcgataagctttaatgcggtagtttatcacagttaaattgctaacgcagtcaggcaccgtgtatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggataacttcgtataatgtatgctatacgaacggtacgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatccttgtaaaacgacggccagtgaattcgagctcggtacccggggatcctctagagtcgacctgcaggcatgcaagcttggcgtaatcatggtcatagctgtttcctgtaccgttcgtataatgtatgctatacgaagttattgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatcacatttccccgaaaagtgccacctgacgtctaagaaaccattattatcatgacattaacctataaaaataggcgtatcacgaggccctttcgtcttcaagaa

SEQ ID NO: 9 - DNA, 20 bp, Artificial Sequence
  gRNAII
gatgaacgtgccggaaagcg

SEQ ID NO: 10 - DNA, 499 bp, Artificial Sequence
  JM108-araBAD-cre(Amp) L-arm
gggggttgttcttcgaacgcctgaatgtatcggtacagaaactggaaaacgccatcgagttttatggcgcgatttcgcataacaaactgcacggcctgtcagtgcaggttgaaaccaatcacgtcaaattaccggcggtggtggacggctttgccagcgaagggatcatccagttctttttcgaagaaacgcaagacgagaatggctttaatatctacattctcgacgaaagcaaccgggttgaggtatatcaccactgcgaaggcagcaaagaggagctggtacgtgacgtcagtcgcttctactcgtcatcgcatgaccgttttacctacggctcaagcttcatcaacttcaacctgccgcagttctatcagattgtgaaggttgatggtcgtgaacaggtgattccgttccgcacaaaatctatcggtaacatgccgcctgccaatcaggatcacgatacgccgctattacagcaatatttttcgtgatgaacgtg

SEQ ID NO: 11 - DNA, 496 bp, Artificial Sequence
  JM108-araBAD-cre(Amp)R-arm
atgcaatcttagcggaaactgactgtttcacccgcctgctgcgtcgccgcctgttccagcaaatcccagaaggtttcgccgctgcgatcacaaatccactcatcgcctttcaggtcaaaatggtagccgccctgtttggttgccagccatacctggtgcagcggctcctggcggttgataatgattttgctgccattctcaaaggtaatggtcagtacgccgccgttgatttcgcagtcgatatcgctgtcgccatcccagtcgtccaggcgttcttcaatggtcagccagagttgatcagccaggcgatgaaattcactgtcgttcattgttgtatcctgtttttaagtgatggcggcagtatagcggcatggggtcagggcttcaaagtttgcacctctgcggctgcgttccggcacgattcatccgtcaccggaataatgatgtctctgtgtagcgaaagatttgtctcttcattagggcgcagttacaccac

SEQ ID NO: 12 - DNA, 90 bp, Artificial Sequence
  Amplification primer of 5.5 kb fragment 5.5kb-F
aagcagcagattacgcgcagaaaaaaaggatctcaagaagatccttgtaaaacgacggccagtgaattcggacattgattattgactagt

SEQ ID NO: 13 - DNA, 90 bp, Artificial Sequence
  Amplification primer of 5.5 kb fragment 5.5kb-R
acgaacggtacaggaaacagctatgaccatgattacgccaagcttgcatgcctgcaggtcgactctagagccatagagcccaccgcatcc

SEQ ID NO: 14 - DNA, 26 bp, Artificial Sequence
  Bacteria detection primer of strain JM108 araBAD-cre after gene editing C4818FK060-6JJF1
agcgtggaagtcttctgttatagcca

SEQ ID NO: 15 - DNA, 27 bp, Artificial Sequence
  Bacteria detection primer of strain JM108 araBAD-cre after gene editing C4818FK060-6JJR1
acctcgcgccaggatggctttatgcac

SEQ ID NO: 16 - DNA, 50 bp, Artificial Sequence
  Amplification primer of flp gene flp-F
tttttttgggatgcaccatcaccatcaccatatgccacaatttgatatat

SEQ ID NO: 17 - DNA, 50 bp, Artificial Sequence
  Amplification primer of flp gene flp-R
ttcagtaacattcacgccggaagtggaattttatatgcgtctatttatgt

SEQ ID NO: 18 - DNA, 93 bp, Artificial Sequence
  Amplification primer of FRT-RFP fragment FRT-F1
cgtaaggagaaaataccgcatcagggaagttcctatactttctagagaataggaacttcggaataggaacttccgctcttccgcttcctcgct

SEQ ID NO: 19 - DNA, 93 bp, Artificial Sequence
  Amplification primer of FRT-RFP fragment FRT-R1
gcgtcagaccccgtagaaaagatcagaagtacctattccgaagttcctattctctagaaagtataggaacttccaggaaacagctatgaccat

SEQ ID NO: 20 - DNA, 20 bp, Artificial Sequence
  Amplification primer of FRT vector FRT-F2
tgatcttttctacggggtct

SEQ ID NO: 21 - DNA, 20 bp, Artificial Sequence
  Amplification primer of FRT vector FRT-R2
cctgatgcggtattttctcc

SEQ ID NO: 22 - DNA, 2258 bp, Artificial Sequence
  Cre specific recombinase araBAD-cre inducible expression system
ttatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggatgcaccatcaccatcaccatatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaa

SEQ ID NO: 23 - DNA, 1434 bp, Artificial Sequence
  pSC101 ori(ts) fragment
tatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgaacgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgg

SEQ ID NO: 24 - DNA, 1272 bp, Artificial Sequence
  Flp gene
ttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagtgcgaagtagtgatcaggtattgctgttatctgatgagtatacgttgtcctggccacggcagaagcacgcttatcgctccaatttcccacaacattagtcaactccgttaggcccttcattgaaagaaatgaggtcatcaaatgtcttccaatgtgagattttgggccattttttatagcaaagattgaataaggcgcatttttcttcaaagctttattgtacgatctgactaagttatcttttaataattggtattcctgtttattgcttgaagaattgccggtcctatttactcgttttaggactggttcagaattcctcaaaaattcatccaaatatacaagtggatcgatcctaccccttgcgctaaagaagtatatgtgcctactaacgcttgtctttgtctctgtcactaaacactggattattactcccagatacttattttggactaatttaaatgatttcggatcaacgttcttaatatcgctgaatcttccacaattgatgaaagtagctaggaagaggaattggtataaagtttttgtttttgtaaatctcgaagtatactcaaacgaatttagtattttctcagtgatctcccagatgctttcaccctcacttagaagtgctttaagcatttttttactgtggctatttcccttatctgcttcttccgatgattcgaactgtaattgcaaactacttacaatatcagtgatatcagattgatgtttttgtccatagtaaggaataattgtaaattcccaagcaggaatcaatttctttaatgaggcttccagaattgttgctttttgcgtcttgtatttaaactggagtgatttattgacaatatcgaaactcagcgaattgcttatgatagtattatagctcatgaatgtggctctcttgattgctgttccgttatgtgtaatcatccaacataaataggttagttcagcagcacataatgctattttctcacctgaaggtctttcaaacctttccacaaactgacgaacaagcaccttaggtggtgttttacataatataccaaattgtggcat

SEQ ID NO: 25 - DNA, 34 bp, Artificial Sequence
  Lox71
taccgttcgtataatgtatgctatacgaagttat

SEQ ID NO: 26 - DNA, 34 bp, Artificial Sequence
  Lox66
ataacttcgtataatgtatgctatacgaacggta

SEQ ID NO: 27 - DNA, 48 bp, Artificial Sequence
  FRT
gaagttcctatactttctagagaataggaacttcggaataggaacttc

SEQ ID NO: 28 - DNA, 1090 bp, Artificial Sequence
  rfp gene
gtacccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacatactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaataacgctgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttatagtcgactcgagcggcc

SEQ ID NO: 29 - DNA, 2579 bp, Artificial Sequence
  pUC57(KanR)
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagatatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 30 - DNA, 64 bp, Artificial Sequence
  pMB1 amplification primer pUC57-short-1-1
ataacttcgtatagcatacattatacgaacggtattgagatcctttttttctgcgcgtaatctg

SEQ ID NO: 31 - DNA, 65 bp, Artificial Sequence
  pMB1 amplification primer pUC57-short-1-2
gctatacgaacggtagaattcggatcccgggcccgtcgactgcagtttccataggctccgccccc

SEQ ID NO: 32 - DNA, 70 bp, Artificial Sequence
  Amplification primer of pUC57(KanR) fragment pUC57-short-2-1
ccgggatccgaattctaccgttcgtatagcatacattatacgaagttattctggccgtcgttttacaacg

SEQ ID NO: 33 - DNA, 50 bp, Artificial Sequence
  Amplification primer of pUC57(KanR) fragment pUC57-short-2-2
gttcgtataatgtatgctatacgaagttatgaagatcctttgatcttttc

SEQ ID NO: 34 - DNA, 2182 bp, Artificial Sequence
  pMF65-loxp precursor empty vector plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagaataacttcgtataatgtatgctatacgaacggtagaattcggatcccgggcccgtcgactgcagtttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaataccgttcgtataatgtatgctatacgaagttatgaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 35 - DNA, 50 bp, Artificial Sequence
  Amplification primer of pMF65-loxp empty vector Short-F
gggatccgaattctaccgttcgtatagcatacattatacgaagttattct

SEQ ID NO: 36 - DNA, 54 bp, Artificial Sequence
  Amplification primer of pMF65-loxp empty vector Short-R
gggcccgtcgactgcagtttccataggctccgcccccctgacgagcatcacaaa

SEQ ID NO: 37 - DNA, 50 bp, Artificial Sequence
  Amplification primer of RFP fragment RFP-F
atgctatacgaacggtagaattcggatcccgtacccaatacgcaaaccgc

SEQ ID NO: 38 - DNA, 50 bp, Artificial Sequence
  Amplification primer of RFP fragment RFP-R
gagcctatggaaactgcagtcgacgggcccggccgctcgagtcgactata

SEQ ID NO: 39 - DNA, 3879 bp, Artificial Sequence
  pMF7-loxp-RFP precursor plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacataacttcgtataatgtatgctatacgaacggtagaattcggatcccgtacccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacatactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaataacgctgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttatagtcgactcgagcggccgggcccgtcgactgcagatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgggtaccgttcgtataatgtatgctatacgaagttattcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacgtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 40 - DNA, 3369 bp, Artificial Sequence
  pMF5-loxp-RFP precursor plasmid
ttctcatgtttgacagcttatcatcgataagctttaatgcggtagtttatcacagttaaattgctaacgcagtcaggcaccgtgtatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcaataacttcgtataatgtatgctatacgaacggtactgcagtgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcggtacccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacatactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaataacgctgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttatagtcgactcgagcggccagtcgacgcatgctaccgttcgtataatgtatgctatacgaagttattgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatcacatttccccgaaaagtgccacctgacgtctaagaaaccattattatcatgacattaacctataaaaataggcgtatcacgaggccctttcgtcttcaagaa

SEQ ID NO: 41 - DNA, 2789 bp, Artificial Sequence
  pMF7-loxp precursor empty vector plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacataacttcgtataatgtatgctatacgaacggtagaattcggatcccgggcccgtcgactgcagatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgggtaccgttcgtataatgtatgctatacgaagttattcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacgtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 42 - DNA, 2289 bp, Artificial Sequence
  pMF5-loxp precursor empty vector plasmid
ttctcatgtttgacagcttatcatcgataagctttaatgcggtagtttatcacagttaaattgctaacgcagtcaggcaccgtgtatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcaataacttcgtataatgtatgctatacgaacggtactgcagtgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcggatcctctagagtcgacgcatgctaccgttcgtataatgtatgctatacgaagttattgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatcacatttccccgaaaagtgccacctgacgtctaagaaaccattattatcatgacattaacctataaaaataggcgtatcacgaggccctttcgtcttcaagaa

SEQ ID NO: 43 - DNA, 784 bp, Artificial Sequence
  pMB1 ori derivative (784 bp)
cgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctt

SEQ ID NO: 44 - DNA, 589 bp, Artificial Sequence
  pMB1 ori (589 bp)
tttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 45 - DNA, 674 bp, Artificial Sequence
  pUC ori (674 bp)
atgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggg

SEQ ID NO: 46 - DNA, 389 bp, Artificial Sequence
  R6K ori(389bp)
tgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatc

SEQ ID NO: 47 - DNA, 3219 bp, Artificial Sequence
  pMF9-FRT precursor empty vector plasmid
ttctcatgtttgacagcttatcatcgataagctttaatgcggtagtttatcacagttaaattgctaacgcagtcaggcaccgtgtatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcagggaagttcctatactttctagagaataggaacttcggaataggaacttccgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatccttgtaaaacgacggccagtgaattcgagctcggtacccggggatcctctagagtcgacctgcaggcatgcaagcttggcgtaatcatggtcatagctgtttcctggaagttcctatactttctagagaataggaacttcggaataggtacttctgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatcacatttccccgaaaagtgccacctgacgtctaagaaaccattattatcatgacattaacctataaaaataggcgtatcacgaggccctttcgtcttcaagaa

SEQ ID NO: 48 - DNA, 35 bp, Artificial Sequence
  attB sequence
ggtgccagggcgtgcccttgggctccccgggcgcg

SEQ ID NO: 49 - DNA, 37 bp, Artificial Sequence
  attP sequence
cccaactggggtaacctttgagttctctcagttgggg

SEQ ID NO: 50 - DNA, 1818 bp, Artificial Sequence
  phiC31( protein ID ADD10761.1)
gtggacacgtacgcgggtgcttacgaccgtcagtcgcgcgagcgcgaaaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgtag